(19) United States
(12) Patent Application Publication
(10) Pub. No.: US 2026/0090984 A1
(43) Pub. Date: Apr. 2, 2026

(54) CONTACT LENSES INCLUDING MEDICAMENTS AND METHODS OF MAKING AND USING SAME INCLUDING SLOW RELEASE OF GLAUCOMA TREATING DRUGS AND COMFORT ENHANCING DRUGS

(71) Applicant: MEDIPRINT OPHTHALMICS INC., San Diego, CA (US)

(72) Inventors: Praful DOSHI, Poway, CA (US); Brandon HUANG, San Diego, CA (US); Stephen HABE, San Diego, CA (US); Neil PATODIA, San Diego, CA (US); Pavanasam BALASUBRAMANIAN, San Diego, CA (US); Tyler HOUCHIN, La Jolla, CA (US)

(73) Assignee: MEDIPRINT OPHTHALMICS INC., San Diego, CA (US)

(21) Appl. No.: 19/411,056

(22) Filed: Dec. 5, 2025

Related U.S. Application Data
(63) Continuation-in-part of application No. PCT/US2024/032518, filed on Jun. 5, 2024.
(60) Provisional application No. 63/506,424, filed on Jun. 6, 2023.

(51) Int. Cl. A61K 9/00; A61K 31/46; A61K 31/5575; A61K 31/728; A61K 38/17; A61K 47/10

(52) U.S. Cl. CPC A61K 9/0051; A61K 31/46; A61K 31/5575; A61K 31/728; A61K 38/179; A61K 47/10

(57) ABSTRACT

The present invention recognizes that medical devices, such as contact lenses, can be made having at least one coating layer provide drug storage and drug release for the treatment of various diseases, disorders, or conditions. One aspect includes a packaged medical device including a medicament. Another aspect includes a drug delivery contact lens. A further aspect includes a method of making a drug delivery contact lens. An additional aspect includes a method of using a drug delivery contact lens. A further aspect includes a medical device including bimatoprost and N-vinylpyrrolidone. An additional aspect includes a contact lens soaking solution. Another aspect includes a method of loading contact lenses. Another aspect includes loaded and refreshed contact lenses. A further aspect includes methods of using loaded and refreshed contact lenses.

### RELATED APPLICATIONS

The present application is a Continuation-in-Part of PCT Application Number PCT/US24/032518, filed Jun. 5, 2024, now pending, which claims benefit of priority to U.S. Provisional Application Ser. No. 63/506,434, filed Jun. 6, 2023, now expired; each of which is incorporated by reference in its entirety herein.

### TECHNICAL FIELD

The present invention generally relates to the fields of medical devices, including but not limited to contact lenses, that include a medicament or drug in a coating layer and methods of making and using such medical devices. The coating layer is preferably made at least in part using printing, preferably but not limited to digital printing.

### BACKGROUND

Medical devices that include a medicament have been known. Examples include contact lenses and stents for the treatment or prevention of a variety of diseases, disorders or conditions, such as contact lenses for the treatment of glaucoma and stents for the treatment or prevention of restinosis. Existing medical devices that include medicaments are traditionally made using relatively simple drug coating or drug impregnation technologies that do not allow the modulated release of the medicament from the coating. The present invention addresses these limitations and provides additional benefits as well.

A variety of medical devices, particularly contact lenses, that include a medicament have been described. For example, U.S. Pat. No. 7,638,137B2 to Chauhan et al. describes drug delivery systems through dispersion of transparently encapsulated drugs within the lens. However, such dispersion inside the lens could alter the physical properties of the polymeric lens materials. Also, while encapsulated drugs may be visually transparent in certain instances, the may interfere with the optical properties of the lens. Also, drugs inside the lens may be released from either or both the anterior and posterior surfaces of the lens and thus not providing the desired dosage of a drug to the cornea or other areas of an eye structure and surrounding tissues. This document also provides a survey of the literature relating to issues relating to drug release.

U.S. published Patent Application No. 2009/07504245A1 to Orilla et al. describe the masking of a color of a drug by applying a color layer on top of the drug. This document does not relate to controlling the drug release rate from the lens.

Also, U.S. published Patent Application No. 2009/0004244 to Orilla et al. describes deposing a drug in an iris simulated pattern to provide a cosmetic appearance of a lens for drug delivery. This document does not relate to how drug release rate can be controlled.

In addition, U.S. Pat. No. 6,887,858 to Yerxa describes formulations for the treatment of dry eye diseases. The document is not related to drug release from a medical device such as a contact lens.

Furthermore, U.S. Pat. No. 6,294,553 to Gil et al. describes a drug for ocular surface pain. Gil et al. does not, however, relate to controlled drug delivery rate.

U.S. Pat. No. 3,786,812 to Neefe describes the use of contact lenses for drug delivery. This document, however, does not relate to achieving the desired release rate of a drug from a lens.

Also, U.S. Pat. No. 3,618,604 to Ness and U.S. Pat. No. 3,828,777 to Ness describe polymeric plastics in which a drug is held to provide controlled drug release rate. The documents, however, do not relate to the ability to adjust drug release rate.

In addition, U.S. Pat. No. 10,463,677 to Esaki et al. generally relates to stabilization of latanoprost by cyclodextrin.

Also, Published US Patent Application No. 2012/0021013 to Esaki et al. generally relates to stabilization of latanoprost by cyclodextrin.

Furthermore, Rodriguez-Aller et al. “New Prostaglandin analog formations for glaucoma treatment containing cyclodextrins for improved stability, solubility and ocular tolerance” European Journal of Pharmaceutics and Biopharmaceuticals, Volume 95, Part B, Pg 203-214 (2015) (Abstract only) https://www.sciencedirect.com/science/article/abs/pii/S093964111500212X?via %3Dihub.

In addition, U.S. Pat. No. 9,539,262 to Khopade et al. generally relates to prostaglandin derivates such as latanoprost formulated with polyethylene glycol hyroxystearate having decreased absorption that was not addressed by addition of oil.

Also, EP 1681059 generally relates to the effects of different formulation conditions such as pH on the degradation of latanoprost.

### BRIEF DESCRIPTION OF THE FIGURES

FIG. 1. generally depicts 30% NVP Semi-log Plots, Full Timescale. The slope of the function is the rate constant of drug release from the lens. A higher magnitude slope indicates a faster release, and a lower magnitude slope indicates a slower release.

FIG. 2. generally depicts 30% NVP Semilog Plots, 24-192 hours. The slope of the function is the rate constant of drug release from the lens. A higher magnitude slope indicates a faster release, and a lower magnitude slope indicates a slower release.

FIG. 3. generally depicts 10-20% NVP Semilog Plots, Full Timescale. The slope of the function is the rate constant of drug release from the lens. A higher magnitude slope indicates a faster release, and a lower magnitude slope indicates a slower release.

FIG. 4. generally depicts 10-20% NVP Semilog Plots 24-192 Hrs. The slope of the function is the rate constant of drug release from the lens. A higher magnitude slope indicates a faster release, and a lower magnitude slope indicates a slower release.

FIG. 5. generally depicts the IOP measurement during the three-week clinical trial of LL-BMT1 26 μg dose. Lenses were inserted on Day 2, Day 9, and Day 16, and the last lens was removed on Day 23.

FIG. 6. generally depicts the IOP measurement during the three-week clinical trial of LL-BMT1 32 μg dose. Lenses were inserted on Day 2, Day 9, and Day 16, and the last lens was removed on Day 23.

FIG. 7. generally depicts a cross-section of the coated HA contact lens. 1 shows the coating on the lens, cured for 1-15 minutes to promote random crosslinking to entrap HA. In addition, 1 contains a PEG monomer, which when polymerized, creates larger pore sizes in the coating to absorb HA from packaging solution. 2 is the coated contact lens. 3 is the thin film of tear/HA formed during contact lens wear from released HA.

FIG. 8. generally depicts Hyaluronic Acid interlaced with the methacrylate polymer matrix. 1: polymer coating matrix. 2: Contact lens with coating and entrapped HA. 4. Hyaluronic Acid dimers entrapped in the coating.

FIG. 9. generally depicts HA Release from MPO-Produced HA-Coated Lenses

FIG. 10. generally depicts HA Release from MPO-Produced HA-Coated Lenses

FIG. 11. generally depicts the effects of Coating a Contact Lens on HA Release

FIG. 12. generally depicts HA Release from MPO-Produced HA-Coated Lenses

FIG. 13. generally depicts HA Release from MPO-Produced HA-Coated Lenses

FIG. 14. generally depicts HA Release from MPO-Produced HA-Coated Lenses

FIG. 15. generally depicts HA Release from MPO-Produced HA-Coated Lenses

FIG. 16. generally depicts HA release profile in terms of its molecular weight from MPO-produced HA-contact lenses.

### SUMMARY

The present invention recognizes that medical devices, such as but not limited to contact lenses, can be made having at least one coating layer provide drug storage and drug release for the treatment of various diseases, disorders, or conditions.

A first aspect of the present invention includes a medical device that incorporates at least one drug in at least one coating.

A second aspect of the present invention includes a method of making a medical device that incorporates at least one drug in at least one coating.

A third aspect of the present invention includes a method of using a medical device of the present invention to treat or prevent a disease, disorder or condition.

A fourth aspect of the present invention includes a packaged medical device including a medicament.

A fifth aspect of the present invention includes a drug delivery contact lens.

A sixth aspect of the present invention includes a method of making a drug delivery contact lens.

A seventh aspect of the present invention includes a method of using a drug delivery contact lens.

An eighth aspect of the present invention includes a medical device including bimatoprost and N-vinylpyrrolidone.

A ninth aspect of the present invention includes a contact lens soaking solution.

A tenth aspect of the present invention includes a method of loading contact lenses.

A eleventh aspect of the present invention includes loaded and refreshed contact lenses.

A twelfth aspect of the present invention includes methods of using loaded and refreshed contact lenses.

A thirteenth aspect of the present invention includes loading of Active Pharmaceutical Ingredient (API) from packaging solution by HA coated contact lens.

### DETAILED DESCRIPTION OF THE INVENTION

### Definitions

Unless defined otherwise, all technical and scientific terms used herein have the same meaning as commonly understood by one of ordinary skill in the art to which this invention belongs. Generally, the nomenclature used herein and the laboratory procedures well known and commonly employed in the art. Conventional methods are used for these procedures, such as those provided in the art and various general references such as U.S. Pat. Nos. 5,160,463; 5,271,874; 5,018,849; 5,034,166; 5,414,477; 6,315,410; 6,899,426B2; 7,638,137B2; US Published Patent Application US2009/0062381A1; Day et al., Current Optometric Information and Terminology, Third Edition, American Optometric Association (1980); Howley's Condensed Chemical Dictionary (1981); Federation of Societies for Coatings Technology; and “Contact Lenses for Drug Delivery: Achieving Sustained Release with Novel Systems,” Alvarez Lorenzo et. al. American Journal of Drug Delivery, (2006) 4 (3) (3) (5). Where a term is provided in the singular, the inventors also contemplate the plural of that term. The nomenclature used herein and the laboratory procedures described below are those well-known and commonly employed in the art. As employed throughout the disclosure, the following terms, unless otherwise indicated, shall be understood to have the following meanings:

“About” when referencing a value, relates to plus or minus 10% of that value.

“Directly” refers to direct causation of a process that does not require intermediate steps.

“Indirectly” refers to indirect causation that requires intermediate steps.

“Digitally Encoded Image” or “Digital Image” refers to an image that has been created or stored in a digital format. A digitally encoded image can be made using methods known in the art, such as artistic renditions or scanning or otherwise translating an image. A digitally encoded image can be stored on appropriate storage medium, such as magnetic medium or polymers such as cyclo-olefin copolymers. A plurality of digitally encoded images can be stored together or separately to form a database of digitally encoded images that are accessible individually or in combination. Such digitally encoded images can be altered using established methods, such as artistic renditions or image modulating software. A plurality of images can also be merged to form a new digitally encoded image.

“Solvent” refers to an aqueous, organic or inorganic solvent, such as water, isopropanol, tetrahydrofuran or acetone.

“Surfactant” refers to a surfactant as that term is known in the art, such as, for example, acetylene glycol or polyoxyethylene alkyl.

“Dispersant” refers to dispersants as they are known in the art, such as, for example, the Tergitol series from Union Carbide, polyoxylated alkyl ethers, alkyl diamino quaternary salts or “Pecegal “O”” from GAF (U.S. Pat. No. 5,560,766). Dispersants are preferably used at between about 0.1% and about 10%, more preferably between about 0.5% and about 5%.

“Lens” as used herein refers to a composition of matter that can transmit light. A lens preferably can act as an optical lens, such as a contact lens. In certain aspects of the present invention, a lens need not act as an optical lens, such as a contact lens that is used for therapeutic purposes as opposed to purposes relating to the correction, improvement or alteration of a user's eyesight.

“Contact Lens” refers to a structure that can be placed on or within a wearer's eye. A contact lens can correct, improve, or alter a user's eyesight, but that need not be the case. A contact lens can be of any appropriate material known in the art or later developed, and can be a soft lens, a hard lens or a hybrid lens. A contact lens can be in a dry state or a wet state.

“Soft Lens” refers to a variety of soft lenses as they are known in the art that are characterized as having, for example, at least one of the following characteristics: oxygen permeable, hydrophilic or pliable.

“Hard Lens” refers to a variety of hard lenses as they are known in the art that are characterized as having, for example, at least one of the following characteristics: hydrophobic, gas permeable or rigid.

“Hybrid Lens” refers to a variety of hybrid lenses as they are known in the art, such as, for example, a lens having a soft skirt and a hard center.

“Dry State” refers to an article of manufacture or a portion thereof in a state prior to hydration or the state of an article of manufacture or a portion thereof under storage or use conditions.

“Wet State” refers to an article of manufacture or a portion thereof in a hydrated state.

“Transparent” refers to a substantial portion of visible light transmitted through a structure, such as greater than or equal to 90% of incident light.

“Opaque” refers to a substantial portion of visible light reflected or absorbed by a structure, such as greater than or equal to 90% of incident light.

“Partially opaque” refers to a combination of transparent and opaque.

“Hydrogel” refers to a polymer that swells in an aqueous solution due to the absorbance of water. A hydrogel includes water or an aqueous solution as part of its structure.

“Polymer” refers to a linkage of monomers. Preferably, a polymer is a polymer appropriate for use in lenses, such as contact lenses. A polymer can be, for example, a homopolymer, a heteropolymer, a copolymer, a hydrophobic polymer, a hydrophilic polymer or any combination thereof.

“Hydrophobic Polymer” refers to a polymer that does not absorb an appreciable amount of water or an aqueous solution (see, U.S. Pat. No. 5,034,166).

“Hydrophilic Polymer” refers to a polymer that absorbs an appreciable amount of water or an aqueous solution (see, U.S. Pat. No. 5,034,166). Lens forming materials that are suitable in the fabrication of contact lenses are illustrated by one or more of the following U.S. Pat. Nos. 2,976,576; 3,220,960; 3,937,680; 3,948,871; 3,949,021; 3,983,083; 3,988,274; 4,018,853; 3,875,211; 3,503,942; 3,532,679; 3,621,079; 3,639,524; 3,700,761; 3,721,657; 3,758,448; 3,772,235; 3,786,034; 3,803,093; 3,816,571; 3,940,207; 3,431,046; 3,542,461; 4,055,378; 4,064,086; 4,062,624; and 5,034,166.

“Hydrophilic Monomer” refers to monomers used to make soft lenses, such as hydroxyethylmethacrylate, methacrylic acid, or N-vinylpyrrolidone (U.S. Pat. Nos. 5,271,874; 5,272,010).

“Hydrophilic Monomer” refers to monomers used to make hard lenses, such as methylmethacrylate, ethoxyethylmethacrylate, styrene, or silicone (U.S. Pat. Nos. 5,271,874; 5,272,010).

“Homopolymer” refers to a polymer comprising a single type of monomer such as hydroxyethylmethacrylate.

“Heteropolymer” refers to a polymer comprising more than one type of monomer such as hydroxyethylmethacrylate and methacrylic acid.

“Copolymer” refers to the use of two different polymers to make a polymer chain.

“Acrylic Polymer” or “Acrylics” refers to a variety of polymer of that genus and species as they are known in the art, such as, for example, hydroxyethylmethacrylate.

“Silicone Polymer” or “Silicones” refers to a variety of polymers of that genus and species as they are known in the art, such as, for example Tris (such as Tris (pentamethyldisiloxyanyl)-3-methacrylate-propylsilane or 3-methacryloxypropy tris(trimethylsiloxy)silane).

“Polycarbonate Polymer” or “Polycarbonate” refers to a variety of polymers of that genus and species as they are known in the art, such as, for example Lexan.

“Initiator” in the context of polymerization refers to an initiator as that term is known in the art, such as, for example, a chemical that starts a polymerization reaction.

“UV Initiator” in the context of polymerization refers to a UV initiator as that term is known in the art, such as, for example, a chemical that becomes reactive or active with the adsorption of energy, such as UV energy, such as, for example benzoin methyl ether.

“Binder” or “bonding agent” refers to compounds used perform the function of increasing the interaction between moieties, such as between monomers and polymers such as those terms are known in the art. Examples of binders or binding agents are hexamethylene diisocyanate or other isocyanate compounds.

“Thickener” refers to a compound that is used to increase the viscosity of a liquid or partially liquid mixture or solution such as that term is known in the art. An example of a thickener is polyvinyl alcohols.

“Anti-kogating agent” or “non-kogating agent” refers to compounds that facilitate printing processes that utilize nozzles, such as such terms are known in the art.

“Dispersant” refers to a surface-active agent added to a suspending medium to promote the distribution and separation of fine or extremely fine solid particles.

“Thermal Initiator” in the context of polymerization refers to a thermal initiator as that term is known in the art, such as, for example, a chemical that becomes active or reactive with the absorption of heat energy, such as, for example, Vazo-64 or azobisisobutyronitrile.

“Anti-Bacterial Agent” refers to a compound or composition that can act as a bactericidal or bacteriostatic or can reduce the growth rate of a bacteria such as tetrabutylammonium chloride.

“Anti-Fungal Agent” refers to a compound or composition that can act as a fungicidal or fungistatic or can reduce the growth rate of a fungi such as benzalkonium chloride salicylic acid.

“Disinfectant” refers to a compound or composition that can reduce the type, number or diversity of microorganisms.

“Humectant” refers to compounds that reduce evaporation, such as ethylene glycol.

“Printing” refers to the application of at least one printing formulation to a surface or structure. Printing can use any appropriate device or method known in the art of later developed for a particular purpose.

“Printing Device” refers to any appropriate device for printing on a surface or structure known in the art or later developed for a particular purpose. Preferably, a printing device includes the dispensation of microdroplets of liquid. The size or volume of the microdroplets can vary, but generally the smaller the microdroplet, the higher the quality of the printing produced. Preferred microdroplets are between about 1 picoliter and about 1,000 microliters, preferably between about 10 picoliters and about 10 microliters or between about 100 picoliters and about 1 microliter. Preferred microdroplets can also be in the microliter range.

“Ink Jet Printing” refers to printing using a printing device that comprises at least one ink jet. Such printing devices are commercially available such as through, for example, Hewlett Packard Corporation (such as DeskJet 560C printer cartridges) and Encad Corporation.

“Piezo Printing” refers to printing using a printing device that comprises at least one piezo printing structure. Such piezo printing structures are known in the art, such as, for example, those available through Packard Instruments and Hewlett Packard Corporation or Canon Inc.

“Thermal Printing” refers to printing using a printing device that comprises at least one thermal printing structure. Such thermal printing structures are known in the art, such as, for example, those available through Hewlett Packard Corporation.

“Laser Printing” refers to printing using a printing device that uses at least one laser printing structure. Such printing structures are known in the art, such as, for example, those available through Cannon or Hewlett Packard Corporation.

“Pad Transfer Printing” refers to printing using a pad transfer printing device. Such pad transfer printing devices are known in the art, particularly for printing in the field of contact lenses. Briefly, a layer is placed or printed on a pad transfer device and the layer on the pad transfer device is transferred to another surface, such as a polymer or lens or other surface (U.S. Pat. No. 3,536,386 to Spivack, issued Oct. 27, 1970; U.S. Pat. No. 4,582,402 to Knapp, issued Apr. 15, 1986; U.S. Pat. No. 4,704,017 to Knapp, issued Nov. 3, 1987; U.S. Pat. No. 5,034,166 to Rawlings et al., Jul. 23, 1991; U.S. Pat. No. 5,106,182 to Briggs et al., issued Apr. 21, 1992; U.S. Pat. No. 5,352,245 to Su et al., issued Oct. 4, 1994; U.S. Pat. No. 5,452,658 to Shell, issued Sep. 26, 1995 and U.S. Pat. No. 5,637,265 to Misciagno et al., issued Jun. 10, 1997).

“Impregnation” refers to a drug being contacted with a surface, such as a polymer, and the drug diffuses into the polymer (EP 0357062 to Pfortner, published Mar. 7, 1990).

“Chemical Bond” refers to a covalent bond or non-covalent bond.

“Polymer-Polymer Bond” refers to two polymers forming covalent or non-covalent bonds, such as by cross linking polymers formed between two polymers, such as hydroxyethyl methylacrylate and ehtyleneglycoldimethacrylate.

“Dry State” refers to a polymer that is not fully hydrated.

“Wet State” refers to a polymer that is fully hydrated.

“Forming a Lens” or “Fabricating a Lens” refers to any method or structure known in the art or later developed used to form a lens. Such forming can take place, for example, using cast-molding, spin-casting, cutting, grinding, laser cutting, stamping, trimming, engraving, etching or the like (U.S. Pat. No. 4,558,931 to Fuhrman, issued Dec. 17, 1985).

“Cast-Molding” in the context of forming a lens refers to the formation of at least a portion lens using a mold (U.S. Pat. No. 3,536,386 to Spivak, issued Oct. 27, 1970; U.S. Pat. No. 3,712,718 to LeGrand et al., issued Jan. 23, 1973; U.S. Pat. No. 4,582,402 to Knapp, issued Apr. 15, 1986; U.S. Pat. No. 4,704,017 to Knapp, issued Nov. 3, 1987; U.S. Pat. No. 5,106,182 to Briggs et al., issued Apr. 21, 1992; U.S. Pat. No. 5,160,463 to Evans et al., issued Nov. 3, 1992; U.S. Pat. No. 5,271,874 to Osipo et al., issued Dec. 21, 1993 and EP 0357062 to Pfortner, published Mar. 7, 1990).

“Spin-Casting” in the context of forming a lens refers to the formation of a lens using centrifugal force (U.S. Pat. No. 3,557,261 to Wichterle, issued Jan. 19, 1971 and U.S. Pat. No. 5,034,166 to Rawlings et al., issued Jul. 23, 1991).

“Information Storage Medium” refers to any medium of expression that can store information in any appropriate format either permanently or transiently. Preferred information storage medium includes paper, electronic medium, magnetic medium or polymers, such as cyclo-olefin copolymers.

“Electronic Medium” refers to information storage medium that can store information in electronic form. For example, electronic medium includes magnetic storage medium, such as diskettes.

“Machine Readable Format” refers to information stored on or within an information storage medium in a form, language or arrangement such that a machine, such as a central processing unit (CPU) can access and use the information.

“Database” refers to a collection of information, such as digital images. The information is preferably provided on or within an information storage medium and can be separate from or integral with a central processing unit.

“Printable formulation” refers to a printable formulation that can be used in conjunction with a printing technology or printing device to provide at least one structure, at least one layer, or a combination thereof, of the present invention.

“Subject” refers to, but is not limited to, a human or non-human primate; a companion animal such as but not limited to a dog, a cat, a bird, a fish, a reptile, an amphibian, a fox, a wolf, a pig, a horse or other companion as is known in the art; laboratory animal, such as, but not limited to a mouse, a rat, a guinea pig, a rabbit, a dog, a cat, a ferret, a pig, or other laboratory animals as is known in the art; working animals such as but not limited to a dog, a horse or other working animals as are known in the art; or any other animal as in known in the art that may be in need of the technology of the present invention or for testing of the technology of the present invention.

“Digital printing” refers to the printing of at least a portion of a layer of the present invention using at least one digital image printing technology.

“3D printing” or “three dimensional printing” refers to the printing of three-dimensional structures using appropriate printing technologies and printers as are known in the art or later developed. 3D printing is useful in the making of parts, products or layers using a computer-driven, additive process, one or more layers at a time. 3D printing can build parts or other structures such as layers, using any appropriate material, such as, but not limited to plastic or metal, directly from CAD drawings or other digital images that have been preferably cross sectioned into may, if not hundreds or thousands of layers. 3D printing provides a faster and less costly alternative to machining, such as but not limited to machining, including but not limited to cutting, turning, grinding and drilling of materials, such as solid materials. Although various techniques are used in 3D printing in the relevant art, 3D printers use method of additive fabrication, that is the building a part or structure one layer at a time, with layers ranging in thickness from about a millimeter to less than 1/1,000 of an inch. The building material can be in any appropriate form, such as, but not limited to a liquid, a power or a sheet of material that is cured by heat, UV light, a chemical reaction or other appropriate method.

Other technical terms used herein have their ordinary meaning in the art that they are used, as exemplified by a variety of technical dictionaries.

### INTRODUCTION

The present invention recognizes that medical devices, such as but not limited to contact lenses, can be made having at least one coating layer provide drug storage and drug release for the treatment of various diseases, disorders, or conditions.

As a non-limiting introduction to the breath of the present invention, the present invention includes several general and useful aspects, including:
1) A medical device including a medicament.
2) A method of making a medical device including a medicament.
3) A method of using a medical device of the present invention to treat or prevent a disease, disorder or condition.
4) A packaged medical device including a medicament.
5) A drug delivery contact lens.
6) A method of making a drug delivery contact lens.
7) A method of using a drug delivery contact lens.
8) A medical device including bimatoprost and N-vinylpyrrolidone.
9) A contact lens soaking solution.
10) A method of loading contact lenses.
11) Loaded and refreshed contact lenses.
12) A method of using loaded and refreshed contact lenses.
13) A thirteenth aspect of the present invention includes loading of Active Pharmaceutical Ingredient (API) from packaging solution by HA coated contact lens.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

### GENERAL ASPECTS OF THE PRESENT INVENTION

### I Medical Devices Including a Medicament

Other technical terms used herein have their ordinary meaning in the art that they are used, as exemplified by a variety of technical dictionaries.

The present invention includes an article of manufacture that includes: a) a medical device including at least one surface; and b) one or more coatings provided on at least a portion of the at least one surface. The one or more coatings can include at least one drug.

The medical device of the present invention can be any known in the art or later developed. The medical device can be implanted within a subject as is the case with many medical devices as they are known in the art such as, for example, cardiac stents, joint replacements such a hip and knee among others, birth control sticks, pacemakers, breast implants, facial implants for reconstructive or cosmetic purposes such as for the cheeks and chin, intrauterine devices (IUD's), pins and mesh and resorbable materials such as known in the art (such as, but not limited to, polylactic acid (PLA)) for bone reconstruction or immobilization, dental implants, filters to entrap blood clots in blood vessels, optical lens replacements for cataract treatment, voice boxes for throat cancer patients and the like.

The medical device of the present invention can also be non-implantable as they are know in the art, such as, for example, contact lenses, dental apparatus, drug patches, transdermal drug patches including but not limited to birth control, Alzheimer's patches, smoking cessation patches, hearing aids, earplugs or other devices inserted into the ear to treat swimmer's ear and ear infections and the like.

The medical device of the present invention can be made of any appropriate material or combination of materials as appropriate for the purpose and location where the medical device will ultimately reside within or on a subject. The choice of materials for the medical device is determinable by one skilled in the art, and there are numerous examples in the prior art for the skilled artisan to follow. For the present invention, it is generally the surface of the medical device on which a coating is provided, but this need not be an exclusive requirement.

### Surface

The surface of a medical device that is to be coated in the manner of the present invention can be of any appropriate material and is usually determined or influenced by the nature of the medical device and where, and how long, it is to be implanted, or not implanted, within or on a subject.

Many medical devices present metal on their surface. Examples include, but are not limited to, bone pins and mesh for bone repair and stabilization. Metals that can be used as a surface include, for example, steal, stainless steel, gold, silver and the like.

Some medical devices present a plastic or polymer on their surface. Examples include but are not limited to contact lenses, IUD's an implantable birth control sticks. There are a wide variety of polymers and plastics available for use in medical devices, which are too numerous to enumerate here. Individual polymers and plastics are discussed further herein, and are intended as a limiting list of such materials.

Other medical devices present partially polymerized polymers during their manufacture, but not necessarily in the final product. The partially polymerized polymers can be used as an intermediate product to facilitate bonding with other components of the device. Examples include, but are not limited to, contact lenses and the like.

Still other medical devices present on their surface polymer matrices. Examples include, but are not limited to, limited to materials that allow for skin or other tissue regenerations, such as for trauma, disease, disorder, condition such as, for example, burn treatment, such as those that contain fibronectin or other structural proteins. The polymer matrix or protein matrix can be any appropriate, such as but not limited to proteins, nucleic acids, and carbohydrates.

In addition, still other medical devices present on their surface silicone, ceramic, glass, carbon (inclusive of nanotubles and graphite) and fabric. Examples include, but are not limited to, breast implants, penal implants, hip replacement parts, knee replacement parts, bandages for burn and trauma wounds, and the like. The silicone, ceramic, glass, carbon (including but not limited to graphite including sheets, carbon nano-structures such as tubes, balls, sheets and other structures) and fabric can be any appropriate and as are realized in the art.

The surface of a medical device can also be pretreated or modified by various processes to, in some instances, clean or otherwise prepare the surface for receiving the coating of the present invention. Some pretreatments may be physical in nature, such as polishing, scarring or scoring, whereas others may be chemical in nature. Preferred chemical process include, but are not limited to, chemical coating, chemical cleaning, chemical texture modification, chemical or electrochemical activation or creation of reactive groups on or within said at least one surface, application of one or more chemicals to said at least one surface, and combinations thereof.

### Drug Reservoir Layer

The drug reservoir layer serves to store a drug for later release from the coating. The drug reservoir layer is preferably porous or otherwise is able to contain a drug for this purpose. In one aspect of the present invention, the drug reservoir layer is solid or semi-solid, such as a gel or sol, which can reversibly entrap a drug for later release. The drug reservoir layer can be provided first without a drug and the drug added at a later step. In the alternative the drug reservoir layer can be provided with a drug in one step. The drug reservoir layer is preferably made using printing technology. The choice of polymer depends on several factors, including, for example, the printing technology to be used to print the drug reservoir layer.

The drug reservoir layer can include a polymer with the characteristics stated above. Preferable polymers include, but are not limited to, polyHEMA, polyGMA, polyvinylalcohol, polyDMA, PMMA (polymethylacrylicacid), polycarbonate, PVP (polyvinylpyrolidone), siloxane, and the like. Depending on the polymer and the printing technology chosen, the polymer can be provided in a monomer state and later polymerized, or in the alternative, provided in a partially polymerized state.

The drug reservoir layer can also include a partially polymerized polymer with the characteristics stated above and can be any as appropriate. Preferable polymers include, but are not limited to polyHEMA, polyGMA, polyvinylalcohol, polyDMA, PMMA (polymethylacrylicacid), polycarbonate, PVP (polyvinylpyrolidone), siloxane, and the like. Depending on the partially polymerized polymer and the printing technology chosen, the partially polymerized polymer can be provided in a monomer state and later partially polymerized, or in the alternative, provided in a partially polymerized state.

The drug reservoir layer can include a polymer matrix with the characteristics stated above and can be any as appropriate. Preferable polymer matrix include, but are not limited to, proteins, nucleic acids, and carbohydrates. Depending on the polymer and the printing technology chosen, the polymer matrix can be provided in a monomer state and later polymerized, or in the alternative, provided in a polymerized state.

In addition, still other materials can be used for the drug reservoir layer, such as, but not limited to silicone, ceramic, glass, carbon (inclusive of nanotubles and graphite) and fabric. The silicone, ceramic, glass, carbon and fabric can be any appropriate and as are realized in the art and the choice generally relates, as with other materials used in the drug reservoir layer, to they physical characteristics such as the ability to accept and retain a drug for later release and the printing technology chosen to print the drug reservoir layer.

Preferable materials for the drug reservoir layer include derivatized oligomers. Preferable derivatized oligomers include, but are not limited to HEMA (mydroxyethylmethylacrylates), DMA (dimethylacrylamides), GMA (glycidolmethylacylates), PVA (polyvinlyalcohols), silicone or siloxane. As with other materials used, the choice of derivatized oligomers depends on the physical characteristics of the material and the printing technology used to make the drug reservoir layer.

If the material used for the drug reservoir layer need to be polymerized and cured, then a polymerization initiator or curing initiator needs to be used. The requirement for a polymerization initiator or curing initiator depends on the particular type of polymer/monomer being utilized and the choice is established in the technology. Preferable polymerization initiator or curing initiators include, but are not limited to at least one of UV cure, thermal cure, room temperature cure, simultaneous printing and UV curing or e-beam.

As set forth in the figures, the drug reservoir layer can release a drug in one or more directions. For example, turning to a contact lens, the drug receiving layer can release drug towards the cornea or towards the eyelid when the contact lens is engaged with the eye. The use of layers, or lack thereof, allows for the design of structures that allow drug to be released in one or both directions.

The material used for the drug receiving layer can be bonded to, permanently bonded to, or not bonded to the surface. Certain materials that can be used for the drug reservoir layer inherently bond or do not bond to a surface, depending on the nature of the surface. As discussed previously, the surface can be modified, such as through chemical medication or other methods or techniques, to allow the drug reservoir layer to chemically bond or react with the drug receiving layer components.

### Drug Receiving Layer

The manufacture of the drug reservoir layer can include the use of a drug receiving layer. In this instance, a drug receiving layer is applied to the surface by an appropriate means or method, such as printing. The drug receiving layer could include or not include a drug at this juncture in time. The drug receiving layer has physical and chemical characteristics to allow the efficient and localized acceptance of a drug applied thereto using appropriate methods, preferably printing. Once the drug receiving layer is applied to the surface, then a drug, or an additional drug, is applied thereto to entrap the drug or additional drug therein for later release.

The drug receiving layer can be of any appropriate material with the appropriate physical and chemical characteristics to obtain a structure with the desired characteristics discussed herein. The drug receiving layer can be a chemical. Preferred materials for the drug receiving layer include, but are not limited to, a highly absorbent polymer such as, but not limited to, a polyvinlylpyrrolidone homopolymer, a polyvinylpyrrolidone copolymer, a polyacrylamide homopolymer, a polyacrylamide copolymer, a polyacrylate homopolymer, a polyacrylate copolymer, a proteinaceous material, a carbohydrate, or a combination thereof.

As there may be other layers applied to the surface prior to the drug receiving layer, the drug receiving layer can be applied to such prior layers using appropriate methods. As with other layers of the coating of the present invention, the drug receiving layer can be provided by any appropriate method, preferably by printing technology.

Where the drug receiving layer includes a polymer, then the drug receiving layer can include a bonding agent or crosslinking agent in order to aid in entrapping or otherwise immobilizing a drug for later release from the drug reservoir layer. Preferable bonding agents include, but are not limited to methylacrylic acid, titanates, and silanes. Preferable crosslinking agents include, but are not limited to HDI, and devivitized oligomers of HEMA, GMA, DMA and PVA, Polyfunctional Aziridine, and multifunctional carbodimide.

In one preferred aspect of the present invention, the drug receiving layer includes a highly absorbent polymer. Preferred highly absorbent polymers include, but are not limited to a polyvinylpyrrolidine homopolymer, a polyvinylpyrrolidone copolymer, a polyacrylamide homopolymer, a polyacrylamide copolymer, a polyacrylate homopolymer, a polyacrylate copolymer, a proteinaceous material, a carbohydrate, or a combination thereof.

The preferred method of application of a drug receiving layer of the present invention is printing technologies and coating technologies. Preferable methods of printing include, but are not limited to direct coating, application of droplets or microdroplets, inkjet printing, soaking, impregnation, spin coating, drip coating, screen coating, silk screen coating, or pad printing such as those methods are known in the art. DRUG

The drug provided in the drug reservoir agent is a matter of choice to one skilled in the appropriate arts depending on the disease, disorder or condition to be treated or prevented, along with the location of the article of manufacture on or with the subject and the nature of the medical device used. For example, drug for the treatment or prevention of glaucoma would be provided with a contact lens, whereas a drug for the treatment or prevention of restinosis would be provided with a stent.

The drug released from the article of manufacture should be of the appropriate amount, duration and dosing in order to be an effective amount to prevent or treat at least one disease, disorder or condition. The amount, duration and dosing of a drug to a particular location for such treatment or prevention is available to one skilled in the art. The present invention allows localized and controlled dosing in terms of the amount and duration of the dose and can allow for the continuous or intermittent release of drug for a regime of drug delivery.

One preferable aspect of the present invention is the delivery of a drug to the eye to treat or prevent or treat diseases, conditions or disorders of the eye. There are drugs known to treat or prevent a variety of diseases and conditions with appropriate regimes of dose, time course of administration, and route of administration. The present invention allows for varying the regime of dose and time course and provides a highly localized route of administration as well. Preferred drugs that are antibiotics useful for treatment of eye infections include, but are not limited to, gentamicin, tobramycin, erythromycin, polytrim, cirproflizacin, viamox, and xymar. Preferred drugs that are used to treat glaucoma include, but are not limited to, timolol, alphagan, axopt, cosopt, lumigan, travatan, xalatan, and combigan. Preferred drugs that are ani-inflammatory that are used to treat diseases, disorders and conditions of the eye include, but are not limited to, perdforte, lotemax, fluromethlone, nevanac, acular and xibrom. Other drugs known in the art to treat or prevent diseases, conditions or disorders of the eye include, but are not limited to pilocarpine, dexamethasone, pilocarpine nitrate, tropicamide, timolol, timolol nitrate, timolol maleate, methyl prednisolone, flurbiprofen, penillin G, gentamicin, ciprofloxacin, tobramycin, sulphacetaminde sodium, indomethacin, hydrocortisone, indomethacin, pilocarpine hydrochloride, ciprofloxacin hydrochloride, insulin, indomethacin, and ketorolac tromethamine, either alone or in combination. (See, for example, Yasmin Sultana, Rahul Jain, Rahul Rathod, Asgar Ali, M. Aqil, Department of Pharmaceutics, Faculty of Pharmacy, Hamdard University, New Delhi 110062, INDIA. “Advances in Ophthalmic Drug Delivery Systems: Part I” By—Apr. 12, 2005, in Latest Reviews Vol. 3 Issue 2, 2005, and Yasmin Sultana, Rahul Jain, Rahul Rathod, Asgar Ali, M. Aqil, Department of Pharmaceutics, Faculty of Pharmacy, Hamdard University, New Delhi 110062, INDIA, “Advances in Ophthalmic Drug Delivery Systems: Part II” By—Apr. 12, 2005, in Latest Reviews Vol. 3 Issue 2, 2005, www.pharmmainfo.net/reviews/advances-opthalmic-drug-delivery-systems-part-ii (Apr. 1-2011) (“Sultana et al. Part II). Sultana et al. Part I and Sultana et al. Part II provide reviews and listings of drugs and combinations thereof to treat or prevent various diseases, conditions and disorders of the eye. The patent literature also provides for ocular drug delivery devices and strategies as provided by Sultana et al. Part I and Sultana et al. Part II. See, for example US patent and US published patent application numbers: U.S. Pat. Nos. 4,925,581; 5,227,372; 5,296,228; 5,480,914; 5,578,638; 5,705,194; 5,888,493; 6,242,442; 6,297,240; 6,316,441; 6,410,045; 6,416,740; 20020071874; 20020197300; 20030017199; U.S. Pat. Nos. 5,837,226; 6,017,875; 6,154,671; 6,217,896; 6,319,240; 6,335,335; 6,410,045; 6,539,251; 6,579,519; 20020026176; 20030147849; 20020064513; 20020114778; 20020119941; 20020197300; 20030175324; 20030185892; 20030191426; and 20040037889.

In one aspect the present invention, the drug is provided in the drug reservoir layer and released from the drug receiving either alone or in combination with other ingredients. Alternatively, the drug can be provided in the drug reservoir layer with such other ingredients and then released from the drug reservoir layer without such other ingredients. In a preferred aspect of the present invention the drug is provided at least in part as a sole active ingredient without any other ingredient association that can alter the activity or deliverability of the at least one drug. That is to say that the drug is provided or released alone and free of other ingredients, such as but not limited to those used for encapsulation, micro-encapsulation or emulsification of a drug.

The drug can be provided or released from the drug receiving layer and coating of the present invention in an encapsulated form. Encapsulation of drugs is known in the art, such as and is within the skill of the ordinary artisan. Preferred encapsulation materials include, but are not limited to: biodegradable polycyanoacrylate, biodegradable poly(alkylcyanoacrylates), biodegradable calcium phosphate, legumin, polysaccharides drafted with polyesters (amphyphilic copolymers), poly(methylidene malonate), gelatin, poly(E-caprolactone), sodium alginate, agarose hydrogel, PMMA, biotinylated poly(ethylene glycol) conjugated with lactobionic acid, poly(vinyl alcohol) hydrogel, biotinylated pullulan acetate, dib loc copolymers and mixtures thereof. Wherein the polycyanoacrylates are preferably, but not limited to: polybutylcyanoacrylate, polyhexylcyanoacrylate, polyethyl-cyano-acrylate, polyisobutylcyanoacrylate and mixtures thereof

The drug can be provided or released from the drug receiving layer and coating of the present invention in a micro-encapsulated form. Micro-encapsulation of drugs is known in the art, such as “Microencapsulation Techniques, Factors Influencing Encapsulation Efficiency: A Review” Jyothi et. al Journal of Microencapsulation, Informa Health Care, Volume 27, Issue 3, P. 187-197, and is within the skill of the ordinary artisan.

The drug can be provided or released from the drug receiving layer and coating of the present invention in a nanoencapsulated with an encapsulation material in nanoparticles. Nanoencapsulation of drugs is known in the art and is within the skill of the ordinary artisan. Non-limiting examples of nanoencapsulation materials include: chitosan nanoparticles, human serum albumin nanoparticles; silica nanospheres, PEG'ylated core-shell nanoparticles, biodegradable PGGA (poly(D,L-lactide-co-glycolide) particles, PLA (poly lactic acid), PGA, PLG (poly-(D,L-glycolide) polymeric nanoparticles, biocompatible gliadin nanoparticles, low pH sensitive PEG stabilized plasmid-lipid nanoparticles, tocopherol derivatives stabilized nano-sized emulsion particles, PLA-PEG nanoparticles, nanoparticles composed of hydrophilic proteins coupled with apolipoprotein E, biodegradable poly(vesiln-caprolactone) nanoparticles, biotinylated poly(ethylene glycol) conjugated with lactobionic acid, carboxylmethyl dextran magnetic nanoparticles and mixtures thereof.

The drug can be provided or released from the drug receiving layer and coating of the present invention in an emulsion, water-in-oil emulsion, an oil-in-water emulsion, or a liposome. Emulsions, water-in-oil emulsions, oil-in-water emulsions and liposomes including drugs is known in the art, such as U.S. Pat. No. 7,638,137 B2, and is within the skill of the ordinary artisan.

The drug of the present invention can take any appropriate form, such as a small molecule or a biologic or biologic mimic as those terms are known in the art. As stated previously, a wide variety of drugs in many forms are known for the treatment or prevention of a disease, disorder or condition. The present invention is not limited to any particular type or classification of drug. The structures of the coating of the present invention can be tailored for the storage and release of any appropriate drug. For example, the porosity of a drug reservoir layer would tend to be greater for a larger molecule, and likewise less so for a small molecule. By way of example, a small molecule would include hormones for hormone replacement therapy or nucleoside analogues as anti-viral agents. Biological drugs and related biological mimics, by way of example, would include the general classifications of enzymes, transport proteins, structural proteins, storage proteins, hormone proteins, receptor proteins, contractile proteins, defensive proteins, cytokines, clotting factors and vaccines. An example of a preferred proteins include, but are not limited to, insulin for the treatment of diabetes and antibodies and monoclonal antibodies for the treatment of infection or for targeted delivery of associated drugs.

In essence, virtually any drug can be useful in the present invention and an enumerated listing is beyond the scope of this document. As way of example, the following is a non-limited and non-exhaustive list of general classifications of drugs useful in the present invention: an anti-inflammatory, an anti-allergy, and antibiotic, a drug for the treatment of glaucoma, a drug for the treatment of macular degeneration, an ophthalmic drug, a hydrophilic drug, a hydrophobic drug, an anti-parasitic drug, a steroid, an antibiotic and a medicament for the treatment of dry eye and a medicament for treatment of eye discomfort.

### Printing

A wide variety of printing technologies are applicable to providing the various layers of the coating of the present invention. The choice of which printing technology to use is a matter of choice for the skilled artisan based on the particular size, shape, thickness and other characteristics of the layer being provided. In addition, as some of the layers are printed in liquid or semi-solid form and then transformed into a solid or semi-solid form by, for example but not limited to polymerization or partial polymerization, the characteristics of the printing liquid or semi-solid is to be taken into account. As a preferred aspect of the present invention, the compositions of Doshi et al., published U.S. application No. 2008/0062381A1, published Mar. 13, 2008, are applicable, particularly when the pigment is optionally present in such formulations, and at least one drug is optionally provided in such formulations.

Preferred printing methods are digital in nature, such as those described by Doshi et al. (U.S. 2008/0062381A1) which is incorporated by reference herein in its entirety, such that they allow for a relatively precise method and means to provide a high quality and well defined print product. As the method and associated device are digital in nature, the printing process is adaptable for computer control and product design. Preferred digital printing methods and structures are discussed herein. As a non-limiting introduction to digital printing methods and devices, the following digital printing methods are preferred: ink jet printing, three dimensional printing (3D printing), piezo printing, thermal printing, laser printing MEMS printing (Micromachined ElectroMechanical System) wherein the printing head or related or associated structures are rotatable or non-rotatable. Generally, but not exclusively, a printing solution of the present invention replaces the ink solution of existing and commercially available printing devices, in particular within the printing cartridge.

Likewise, preferred printing methods include pad printing as those methods are known in the art, including but not limited to pad transfer printing. Pad printing is not as exact as digital printing, but is a preferred method of printing for the present invention. Pad printing is known in the art for printing of images of the iris of the eye on contact lenses (see, for example, U.S. Pat. Nos. 5,302,978, 5,414,477, and 4,668,240).

Ink jet printing is known in the art and can take various forms and associated structures as are discussed herein. Generally, ink jet printing refers to printing devices and methods that utilize highly precise printing methods and structures that allow for the production of high quality and precise structures. Generally, available inkjet printing devices and structures can be utilized with minimal modification, with the ink solutions normally present in the ink jet cartridge or reservoir is replaced with a solution that includes a polymerizable monomer and associated polymerization initiators as needed. The polymerizable monomer can be polymerized at will and at a rapid rate after being dispensed from the ink jet printing structure.

Three dimensional printing is based primarily, but not exclusively, on ink jet printing technologies. These methods and devices allow for the generation of one-off or multiple copies of structures. Generally, a polymerizable solutions is placed within the printing device and is dispensed under computer control and polymerized in repeated printing cycles or steps to generate a three dimensional structure. Examples of available and preferred 3D printing devices and related structures and cartridges include, but are not limited to, those disclosed herein and otherwise known in the art or later developed.

Piezo printing is a subtype of ink jet printing that is a preferable printing method of the present invention. Examples of available and preferred piezo printing devices and related structures and cartridges include, but are not limited to, those disclosed herein and otherwise known in the art or later developed.

Thermal printing is a subtype of ink jet printing that is a preferable printing method of the present invention. Examples of thermal printing devices and related structures and cartridges include, but are not limited to, those disclosed herein and otherwise known in the art or later developed.

Laser printing is a subtype of ink jet printing that is a preferable printing method of the present invention. Examples of laser printing devices and related structures and cartridges include, but are not limited to those disclosed herein and otherwise known in the art or later developed.

Optionally, an ink jet printing device can include a rotating printer head that can allow for enhanced printing on curved surfaces.

Another preferred printing method is MEMS printing, wherein MEMS stands for Micromachined electromechanical system and is based on technologies that allow for the printing of integrated circuit boards, but are applicable to the production of very small structures that have functionality. Examples of structures having functionality made by MEMS printing include mechanical gears and other mechanical devices, lab on a chip structures for the performance of laboratory procedures including chemical reactions and diagnostic procedures.

### Modulation of Release of Drug

The combination of the components of the coating of the present invention, in particular the at least one drug reservoir layer that includes at least one drug allows for the controlled release of the at least one drug from the coating. The coating structure allows for the production of a coating layer that can particularly tailor the release of the at least from drug from the coating layer for desirable characteristics, such as, but not limited to, dose, regime, time course of delivery and route of administration. As the article of manufacture can be localized to a particular locus on a subject, the drug can be delivered with particular focus with a particular regime, which can allow for less drug being administered to a subject if it were otherwise administered in a more systematic route of administration. The particular physical chemistry phenomenon associated with the release of the drug from the coating layer are discussed herein, but the listing is not to be considered limiting.

In one aspect of the invention, the release of the at least one drug from the coating layer can be modulated by diffusion out of the drug reservoir layer. Determination of the effect of diffusion on the migration of a chemical entity out of a substrate can be made using established methods, formulas and through routine experimentation.

In another aspect of the invention, the release of the at least one drug from the coating layer can be modulated by diffusion out of the drug reservoir layer. Determination of the effect of diffusion on the migration of a chemical entity out of a coating layer of the present invention can be made using established methods, formulas and through routine experimentation.

In another aspect of the invention, the release of the at least one drug from the coating layer can be modulated by mass action out of the drug reservoir layer. Determination of the effect of mass action on the migration of a chemical entity out of a coating layer of the present invention, can be made using established methods, formulas and through routine experimentation.

In yet another aspect of the invention, the release of the at least one drug from the coating layer can be modulated by a concentration gradient of the at least one drug out of the drug reservoir layer. Determination of the effect of a chemical gradient on the migration of a chemical entity out of a coating layer of the present invention, can be made using established methods, formulas and through routine experimentation.

In yet another aspect of the invention, the release of the at least one drug from the coating layer can be modulated by the solubility of the at least one drug in an environment out of the drug reservoir layer. Determination of the effect of a solubility on the migration of a chemical entity our of a coating layer of the present invention, can be made using established methods, formulas and through routine experimentation.

In yet another aspect of the invention, the release of the at least one drug from the coating layer can be modulated by the temperature at which the article of manufacture is held (either at storage temperature or during use) of the at least one drug out of the drug reservoir layer. Determination of the effect of temperature on the migration of a chemical entity out of a coating layer of the present invention, can be made using established methods, formulas and through routine experimentation.

In yet another aspect of the invention, the release of the at least one drug from the coating layer can be modulated by the molecular weight of the at least one drug out of the drug reservoir layer. Determination of the effect of molecular weight on the migration of a chemical entity out of a coating layer of the present invention, can be made using established methods, formulas and through routine experimentation.

In yet another aspect of the invention, the release of the at least one drug from the coating layer can be modulated by a concentration gradient of the at least one drug out of the drug reservoir layer. Determination of the effect of the migration of a chemical gradient on a chemical entity out of a coating layer of the present invention, can be made using established methods, formulas and through routine experimentation.

In further aspect of the invention, the release of the at least one drug from the coating layer can be modulated by the thickness of the coating layer, and the components thereof, namely the drug reservoir layer. Determination of the effect of the thickness of the coating and the components thereof on the migration of a chemical entity out of a coating layer of the present invention, can be made using established methods, formulas and through routine experimentation.

In a still further aspect of the invention, the release of the at least one drug from the coating layer can be modulated by the porosity of the coating layer, and the components thereof, namely the drug reservoir layer. Determination of the effect of the porosity of the coating and the components thereof on the migration of a chemical entity out of a coating layer of the present invention, can be made using established methods, formulas and through routine experimentation.

In a still further aspect of the invention, the release of the at least one drug from the coating layer can be modulated by the pore size of the coating layer. Determination of the effect of the pore size of the coating layer and the components thereof on the migration of a chemical entity out of a coating layer of the present invention, can be made using established methods, formulas and through routine experimentation.

In a still further aspect of the invention, the release of the at least one drug from the coating layer can be modulated by the molecular exclusion size of the coating layer, and the components thereof, namely the drug reservoir layer. Determination of the effect of the molecular exclusion size of the coating and the components thereof on the migration of a chemical entity out of a coating layer of the present invention, can be made using established methods, formulas and through routine experimentation.

In another aspect of the invention, the release of the at least one drug from the coating layer can be modulated by the water content of the coating layer, and the components thereof, namely the drug reservoir layer. Determination of the effect of the water content of the coating and the components thereof on the migration of a chemical entity out of a coating layer of the present invention, can be made using established methods, formulas and through routine experimentation.

In yet another aspect of the invention, the release of the at least one drug from the coating layer can be modulated by the concentration of the drug in the coating layer, and the components thereof, namely the drug reservoir layer. Determination of the effect of the concentration of the drug in the coating and the components thereof on the migration of a chemical entity out of a coating layer of the present invention, can be made using established methods, formulas and through routine experimentation.

In a further aspect of the invention, the release of the at least one drug from the coating layer can be modulated by the concentration of the drug in the coating layer, and the components thereof, namely the drug reservoir layer. Determination of the effect of the concentration of the drug in the coating and the components thereof on the migration of a chemical entity our of a coating layer of the present invention, can be made using established methods, formulas and through routine experimentation.

In a still further aspect of the invention, the release of the at least one drug from the coating layer can be modulated by the packaging environment of the coating layer (such as the concentration of drug in the packaging solution, if present), and the components thereof, namely the drug reservoir layer. Determination of the effect of the packaging environment of the coating and the components thereof on the migration of a chemical entity our of a coating layer of the present invention, can be made using established methods, formulas and through routine experimentation.

In one aspect of the invention, the drug can exhibit sustained release over time from the coating layer. This can be achieved by first establishing the relationship of release rate of a given drug for a given material of layer in terms of thickness variation, drug solubility, concentration. In another aspect of the invention, the drug can exhibit intermittent release over time from the coating layer.

In yet another aspect of the invention, more than one drug can be released from the coating layer of the present invention. In the alternative, more than one drug can be provided in a single drug reservoir layer.

### Contact Lens

In one preferred aspect of the present invention, the medical device includes a contact lens. Contact lenses that include a drug, on the surface of the contact lens or within the contact lens are known in the art. However, these contact lenses do not provide the structures of the present invention, such as the at least one coating that includes at least one drug reservoir layer that can include at least one drug, and at least one layer that can include structures, wherein the release of the at least one drug from the at least one coating layer is modulated.

A variety of materials are known in the art for making contact lenses and are useful in the present invention. Preferred materials include, but are not limited to, acrylics, silicones, polyvinylalcohols, and combinations thereof.

There are a variety of general types of contact lenses known in the art and are useful in the present invention. Preferred general types of contact lenses include, but are not limited to hybrid lenses, hydrophilic lenses and hydrophilic lenses.

In addition, there are other general types of contact lenses known in the art and are useful in the present invention. These lenses include, but are not limited to spherical lenses, toric lenses, multifocal lenses, tinted lenses, corrective optical power lenses and lenses without corrective optical power.

There are a variety of methods used to make lenses that are useful in the present invention. Preferred methods of making, at least in part or in combination, contact lenses include, but are not limited to, lathing, cast molding, spin casting and ink jet printing.

Once a contact lens is manufactures, a variety of secondary or finishing operations can be utilized and are useful in the present invention. Preferred secondary or finishing operations include, but are not limited to edging, polishing, tinting, hydration, extraction, and sterilization.

In one aspect of the present invention, the at least one drug in an at least one coating layer can be provided on the surface of a contact lens. In another aspect of the present invention, the at least one drug in at least one coating layer can be provided within a contact lens. In another aspect of the present invention, the at least one drug can be provided inside a contact lens without the structures in an at least one coating layer in combination with at least one drug in at least one coating layer on the surface of a lens. In yet another aspect of the present invention, the at least one coating layer with at least one drug can be provided both on the surface of the lens and inside the lens.

In some cases, drugs provided within the at least one coating can have optical properties that can interfere with the optical function of the contact lens, such as drugs having coloring or opaqueness. Preferred drugs for use in the present invention do not have such optical properties, but that need not be the case as drugs having such optical properties are useful in the present invention.

In another aspect of the present invention, the one or more coatings can optionally dispersed therein nanoparticles having a particles size less than about 50 nm, a nanoencapsulated ophthalmic drug from which the ophthalmic drug is able to diffuse into and migration through the contact lens and into the post-lens tear film or towards the eyelid when the contact lens is placed on the eye, the nanoparticles being disperse within the contact lens or on at least one surface of the contact lens in an amount such that the lens optionally remains substantially optically transparent (see, for example, U.S. Pat. No. 7,638,137B2 to Chauhan et al., issued Dec. 29, 2009).

In another aspect of the present invention, the one or more coatings can optionally dispersed therein nanoparticles having a particles size less than about 50 nm, a nanoencapsulated ophthalmic drug from which the ophthalmic drug is able to diffuse away from and migrate away from the contact lens and into the post-lens tear film or towards the eyelid when the contact lens is placed on the eye, the nanoparticles being disperse within the contact lens or on at least one surface of the contact lens in an amount such that the lens optionally remains substantially optically transparent (see, for example, U.S. Pat. No. 7,638,137B2 to Chauhan et al., issued Dec. 29, 2009).

In yet another aspect of the present invention, when the at least one drug is provided with or without a drug delivery compositions as described herein, the at least one drug as provided with or without a drug delivery compositions is substantially optically transparent. However, this need not be the case. In one aspect of the present invention, when the at least one drug as provided with or without a drug delivery composition is substantially optically transparent or is not substantially optically transparent, the optical characteristics of the at least one drug, or other structures of the at least one coating layer, can be masked with opaque material or tinting, such as color tinting as is known in the art.

### Packaging

The article of manufacture of the present invention can be provided in a variety for forms and packaging formats and solutions as present. Many of these packaging form and formats are established packaging formats, whereas others are unique to the present invention.

The article of manufacture of the present invention can be provided in a packaging in a dry state, preferably in a dehydrated state or a lyophilized state using methods know in the art. The article of manufacture of the present invention can also be provided in a packaging in a wet state, that is to say provided in an appropriate solution and, as appropriate, in a hydrated state.

The format of the packaging can be any as is appropriate. For example, the article of manufacture can be provided in packaging that is appropriate and normal for the article of manufacture, such as vials, other containers such as boxes or plastic containers, or in vials. Vials and blister packaging are preferable, but not necessary, for example, for contact lenses.

The solution present, if any, in a packaging format, in particular for a wet state packaging format can include the at least one drug present in the at least one coating layer, a different drug that that provided in the coating layer, or a combination thereof.

In one instance, the concentration of the drug in a packaging solution is less than the concentration of the drug in the coating layer. In that case, it is likely that the drug in the coating layer may migrate from the coating layer into the packaging layer and eventually reach a steady state equilibrium state, but that not be the case.

In another instance, the concentration of the drug in a packaging solution is equal to the concentration of the drug in the coating layer. In that case, it is likely that the drug in the packaging solution will be in steady state with the drug in the coating layer, but that need not be the case.

In the alternative, the concentration of the drug in the packaging solution is greater than the concentration of the drug in the coating layer. In that case, it is likely that the drug in the packaging solution would migrate into the coating layer and eventually reach a steady state equilibrium state, but that need not be the case.

In yet another instance, a drug provided in the packaging layer that is not present in the coating layer may be present. In that case, it is likely that the drug in the packaging solution would migrate into the contact lens and eventually reach a steady state equilibrium state, but that need not be the case.

### II Methods of Making Medical Devices Including a Medicament

The present invention also includes a method of making an article of manufacture, comprising: a) providing a medical device including at least one surface; b) depositing one or more coatings on at least a portion of the at least one surface, wherein the one or more coatings includes; 1) at least one drug reservoir layer deposited at least in part by printing on the at least one surface, wherein the at least one drug reservoir layer comprises at least one drug.

The present invention also includes a method of making an article of manufacture, including: a) providing a medical device including at least one surface; b) depositing one or more coatings on at least a portion of the at least one surface.

Having discussed the particular structures of the present invention, what they are made of, how they are preferably made, how they interact, how they are assembled and how they are chosen based on their physical and chemical nature, and the like, the discussion now turns to how the article of manufacture is made, with exemplary and preferred examples later provided in the examples section.

### Medical Device

First, a medical device is chosen on which a coating is to be provided. Essentially any medical device can be used in the present invention. The choice of the medical device is one within the skill of the ordinary artisan and the state of the art provides vast literature on a wide variety of medical devices and where they are to be implanted and which drugs would be useful to be provided with a coating of the present invention to treat or prevent any number of diseases, conditions or disorders that a subject may suffer from.

The medical device can be implantable or non-implantable as those terms are known in the art and have been previously discussed. In one preferred aspect of the present invention, the medical device includes a cardiac stent or joint replacement apparatus, or other implantable medical device. In another preferred aspect of the present invention, the medical device includes a contact lens or skin patch drug delivery medical device, or other non-implantable medical device.

### Surface

The medical device presents a surface upon which a coating of the present invention is to be made. The surface of the medical device chosen is usually an inherent property of the medical device, but that need not be the case. The surface can be modified by any number of methods or techniques and known in the art and discussed herein, including chemical modification or physical modification.

In certain preferred aspects of the present invention, as discussed herein, the surface presented for the application of a coating of the present invention includes, but is not limited to, at least one metal, at least one plastic, at least one polymer, at least one partially polymerized polymer, at least one polymer matrix, at least one protein matrix, at least one silicone, at least one ceramic, at least one glass, at least one carbon containing compound, at least one fabric, or a combination thereof.

In other preferred aspects of the present invention, as discussed herein, the surface presented for the application of a coating of the present invention can be modified by a variety of methods before a coating of the present invention is applied thereto. Preferred surface modification methods include but are not limited to one or more chemical processes or one or more physical processes. Preferred chemical processes include, but are not limited to, chemical coating, chemical cleaning, chemical texture modification, chemical or electrochemical activation or creation of reactive groups on or within said at least one surface, application of one or more chemicals to said at least one surface, and combinations thereof. Preferred physical processes include but are not limited to, etching, scoring, spraying of materials on the surface, sputtering of materials on the surface, corona treatment, and combinations thereof.

### Drug Reservoir Layer

The coating of the present invention includes a drug reservoir layer, which includes at least one drug for later release into or onto a subject at the locus where the medical device is provided to a subject. The drug reservoir layer is preferably provided directly on at least a portion of the surface of a medical device as discussed herein and is the first component of the coating of the present invention. However, at least one layer may be provided before an at least one drug reservoir layer in certain aspects of the invention where the direction of release of a drug from a coating of the present invention is desired, such as the case where a medical device presents multiple surfaces for release of a drug from a coating of the present invention, such as, for example, contact lenses where the drug can be released towards the eye, towards the eyelid, or both.

The drug reservoir layer can be made of any appropriate material or combination of materials, and the choice of material is generally within the skill of the art as influenced by a variety of factors, including but not limited to the printing method to be used to provide the drug reservoir layer, the size, thickness an shape of the drug receiving layer desired, the physical and chemical properties desired for the drug reservoir as influenced by the chemical and physical characteristics of the drug provided in the drug receiving layer such that the drug can be released at a desired rate, and the like.

Preferred materials for the drug receiving layer include, but are not limited to, at least one polymer, at least one partially polymerized polymer, at least one polymer matrix, at least one protein matrix, at least one silicone, at least one ceramic, at least one glass, at least one carbon containing compound, at least one fabric or a combination thereof. Other preferred materials include, but are not limited to, derivatized oligomers, such as but not limited to, HEMA, DMA, GMA, PVA, silicone and siloxane, or combinations thereof.

In certain aspects of the present invention, during the printing process used to make the drug reservoir layer, a non-polymerized or partially polymerized printing formulation, which can include at least one drug, is applied to the surface. In that instance, the non-polymerized or partially polymerized formulation is to be polymerized or otherwise cured to stabilize the drug receiving layer and, in certain aspects of the invention, serves to entrap or otherwise localize a drug in the drug reservoir layer for later release therefrom. Preferred methods for polymerizing or curing a drug reservoir when needed or desirable include, but not limited to, at least one UV curing or polymerization, at least one thermal curing or polymerization, at least one room temperature curing or polymerization, at least one simultaneous printing and curing or polymerization, at least one e-beam curing or polymerization, or combinations thereof.

In certain aspects of the present invention, the drug reservoir layer is bonded to, permanently bonded to, or is not bonded to the surface. In this instance, reactive groups on the surface or the drug receiving layer may chemically or physically interact to form chemical bonds, such as covalent bonds, or physical bonds, such as short-range interactions, such as but not limited to hydrogen bonds, van der Walls interactions, hydrophobic interactions, hydrophilic interactions, ionic interactions and the like. The formation of these chemical or physical interactions is dependent upon the chemical nature of the surface and the drug reservoir layer and can be determined by the artisan based on based on the state of the art.

In another aspect of the present invention, as discussed herein, the drug receiving layer can release a drug in one or more directions. In certain cases, the drug receiving layer, based on the nature of the medical device and surface, can release a drug only in one direction as the surface will prevent, or block, the release of drug in one direction as the drug is not able to substantially migrate into the surface or medical devices based on the material presented. As discussed herein, a blocking layer may be provided to prevent a drug from migrating in one direction. As discussed herein, a drug may be released in more than one direction, such as the case of contact lenses.

### Drug Receiving Layer

In one aspect of the present invention, the at least one drug reservoir includes an at least one drug receiving layer. In this aspect of the present invention, the drug receiving layer is printed on the surface, as the drug reservoir layer with at least one drug is as described herein, and an at least one drug is provided to said at least one drug receiving layer to form a drug reservoir layer. The drug is provided to the drug receiving layer my any appropriate method, such as by printing as described herein, but other methods of proving a drug to a drug receiving layer can be used, such as, but not limited to, soaking, dipping and spin coating. As with other layers of the coating of the present invention, the drug receiving layer can be made of any appropriate material or combination of materials, and the choice of material is generally within the skill of the art as influenced by a variety of factors, including but not limited to the printing method to be used to provide the drug receiving layer, the size, thickness an shape of the drug receiving layer desired, the physical and chemical properties desired for the drug reservoir as influenced by the chemical and physical characteristics of the drug provided in the drug receiving layer such that the drug can be released at a desired rate, and the like.

In one aspect of the present invention, the at least one drug reservoir layer includes a chemical coating applied to the surface. In the alternative, the at least one drug receiving layer is applied to another layer that has been previously applied to the surface, such as, but not limited to, a layer to produce a coating layer that released a drug in a particular directions from the coating as described herein.

In another aspect of the present invention, the printing formulation used to print the drug receiving layer can include materials, such as chemicals, to allow for the polymerization or curing of the printed drug reservoir layer, and in certain instances, to allow for the tailoring of the physical characteristics of the drug receiving layer that affect the release of the drug therefrom as described herein, such as, but not limited to porosity, diffusion rate of a drug, and the like. The materials used to obtain these objectives include, but are not limited to bonding agents, cross linking agents, or a combination thereof. The use of bonding agents, cross linking agents, or combinations thereof to provide materials with desirable physical characteristics for the present invention are known in the art and are replete in the literature and adaptation to the present invention can be made using experimentation or mathematical modeling.

In one preferred aspect of the present invention, the drug receiving layer includes a highly absorbent polymer. Preferred highly absorbent polymers include, but are not limited to, at least one polyvinylpyrrolidine homopolymer, at least one polyvinylpyrrolidone copolymer, at least one polyacrylamide homopolymer, at least one polyacrylamide copolymer, at least one polyacrylate homopolymer, at least one polyacrylate copolymer, at least one proteinaceous material, at least one carbohydrate, or a combination thereof.

The drug reservoir can be applied to a surface or desired location using any appropriate method or means as described herein or as known in the art. Preferred methods or means include but are not limited to, direct coating, application of droplets or microdroplets, ink jet printing, soaking, impregnation, spin coating, drip coating, screen coating, silk screen coating, pad printing, or a combination thereof.

### Drug

As discussed previously, the at least one drug reservoir layer of the at least one coating of the present invention includes at least one drug provided therein such that the at least one drug can be released from the at least one coating. In general, the choice of drugs to be provided in the coating layer are a matter of choice for the artisan, and there is a vast body of literature, both patent and not patent, available to the artisan to identify drugs that are effective to treat or prevent an disease, disorder or condition.

The drug can be provided in the coating in an amount sufficient such that when the drug is released from the coating it is provided in a therapeutically effective amount for the route of administration and location of the medical device of the present invention within or on the subject. The physical characteristics of the coating of the present invention as discussed herein, such as, but not limited to, pore size and water content, can be taken into account when considering what concentration of drug to be provided in the coating of the present invention such that the appropriate amount of drug is released from the coating of the present invention.

As discussed herein, a medical device of the present invention is provided within or on a subject such that the drug is released at a particular locus rather than systemically as with other drug delivery methods, such as through injection or oral administration. This allows for the drug to be delivered at a particular location and preferably at a lower or more precise dose than would otherwise be obtainable. The focused delivery of a drug by the medical device of the present invention also would reduce the instance of side effects of drugs that more systemic routs of administration would be characterized because the total body load of a drug in a subject would be greatly reduced compared to more systemic administration of a drug.

As discussed herein, the location of the drug delivery device is determinable by the nature of the medical device and the disease, disorder or condition to be prevented or treated. For example, implantable cardiac stents would be provided in blood vessels as is the normal course of treatment, and contact lenses would normally be provided on the eye, but this need not be the case.

The drug can be provided with the coating layer of the present invention or released from the coating layer of the present invention in a variety of forms. In one aspect of the present invention, the drug is provided in the coating layer or released from the coating layer at least in part as a sole active ingredient without any other ingredient association that can alter the activity or deliverability of said at least one drug. That is to say, the drug is provided or released in a free state and not associated with other chemical entities, such as drug delivery chemical entities as described herein or known in the art.

In the alternative, the drug is provided in the coating layer or released from the coating layer at least in part in at least one encapsulated form, at least one micro-encapsulated form, at least one nano-encapsulated form, in at least one emulsion, in at least one water-in-oil emulsion, in at least one oil-in-water emulsion, or in at least one liposome, or a combination thereof, as described herein or as known in the art.

As described herein the drug provided in the coating layer or released therefrom can be virtually any drug, including but not limited to small molecule drugs or biological drugs as they are known in the art. There is a vast body of literature, both patent literature and non-patent literature for these types of drugs. A comprehensive list is beyond the scope of this document. Preferred classes of drugs are provided herein, and include, but are not limited to, at least one anti-inflammatory drug, at least one anti-allergy drug, at least one antibiotic drug, at least one drug for the treatment of glaucoma, at least one drug for the treatment of macular degeneration, at least one ophthalmic drug, at least one hydrophilic drug, at least one hydrophobic drug, at least one anti-parasitic drug, at least one steroid drug, at least one medicament for the treatment of dry eye and at least one medicament for treatment of eye discomfort, or a combination thereof.

In one preferred aspect of the present invention, the drug is provided in a coating layer or released from the coating layer in an at least one encapsulated form. Preferred encapsulation materials are discussed herein and are known in the art, and include, but are not limited to at least one biodegradable polycyanoacrylate, at least one biodegradable poly(alkylcyanoacrylates), at least one biodegradable calcium phosphate, at least one legumin, at least one polysaccharides drafted with polyesters (amphyphilic copolymers), at least one poly(methylidene malonate), at least one gelatin, at least one poly(E-caprolactone), at least one sodium alginate, at least one agarose hydrogel, at least one PMMA, at least one biotinylated poly(ethylene glycol) conjugated with lactobionic acid, at least one poly(vinyl alcohol) hydrogel, at least one biotinylated pullulan acetate, at least one dibloc copolymers and combinations thereof.

In another preferred aspect of the present invention, the polycyanoacrulate are those disclosed herein or known in the art, including but not limited to, at least one polybutylcyanoacrylate, at least one polyhexylcyanoacrylate, at least one polyethyl-cyano-acrylate, at least one polyisobutylcyanoacrylate and combinations thereof.

In one preferred aspect of the present invention, the drug is provided in a coating layer or released from the coating layer in a nanoencapsulated form with a least one encapsulation material in nanoparticles, a least one oil-in-water emulsion, at least one water-in-oil emulsion or at least one liposome material, or a combination thereof. The nanoparticles, when present, can be any disclosed herein or described in the art, including but not limited to, chitosan nanparticle, human serum albumin nanoparticle; silica nanospheres, PEG'ylated core-shell nanoparticles, biodegradable PGGA (poly(D,L-lactide-co-glycolide) particles, PLA (poly lactic acid), PGA, PLG (poly-(D,L-glycolide) polymeric nanoparticles, biocompatible gliadin nanoparticles, low pH sensitive PEG stabilized plasmid-lipid nanoparticles, tocopherol derivatives stabilized nano-sized emulsion particles, PLA-PEG nanoparticles, nanoparticles composed of hydrophilic proteins coupled with apolipoprotein E, biodegradable poly(vesiln-caprolactone) nanoparticles, biotinylated poly(ethylene glycol) conjugated with lactobionic acid, carboxylmethyl dextran magnetic nanoparticles and combinations thereof.

### Printing

One aspect of the present invention is that the various components of the at least one coating are preferable made using at least one printing technology. The components of the coating include, but are not limited a variety of layers, including but not limited to, and may not include all of the listed components, at least one drug reservoir layer, at least one drug receiving layer, and at least one layer. The same or different printing technologies can be used to make the various components. Likewise, one or more printing technologies can be used to make a particular component. The printing of the various components, or layers, preferably uses a printing formulation of the present invention, but that need not be the case. Printing formations of the present invention are described in further detail herein.

A wide variety of printing technologies are applicable to providing the various layers of the coating of the present invention. The choice of which printing technology to use is a matter of choice for the skilled artisan based on the particular size, shape, thickness, printing resolution and other characteristics of the layer being provided. One skilled in the art would have available technical literature to match the desired characteristics of the layer to be printed with the characteristics, benefits and limitations of a printing technology. Likewise, one skilled in the art would be able to match a printing formation used to make a layer of the present invention with a particular printing technology, and the desired characteristics of the layer to be printed as well.

The characteristics of the printing formulation being used to make the layer, such as, but not limited to the viscosity and surface tension of the printing formation. Also, the nature of the printing device in combination with the printing formation is a factor to consider, such as the case when a printing technology, such as but not limited to inkjet printing technology utilize printing structures that may require relatively stringent physical and chemical characteristics of the printing solution such that the printing formulation does not clog or otherwise damage or interfere with the printing device.

In addition, as some of the layers are printed in liquid or semi-solid form and then transformed into a solid or semi-solid form by, for example but not limited to polymerization or partial polymerization, the characteristics of the printing liquid or semi-solid is to be taken into account. As a preferred aspect of the present invention, the compositions of Doshi et al., published U.S. application No. 2008/0062381A1, published Mar. 13, 2008, are applicable, particularly when the pigment is optionally present in such formulations, and at least one drug is optionally provided in such formulations.

Preferred printing methods are digital in nature, such as those described by Doshi et al. (U.S. 2008/0062381A1) which is incorporated by reference herein in its entirety, such that they allow for a relatively highly precise method and means to provide a high quality and well defined print product. As the method and associated device are digital in nature, the printing process is adaptable for computer control and product design. Preferred digital printing methods and structures are discussed herein. As a non-limiting introduction to digital printing methods and devices, the following digital printing methods are preferred: ink jet printing, three dimensional printing (3D printing), piezo printing, thermal printing, laser printing MEMS printing, wherein the printing head or related or associated structures are rotatable or non-rotatable. Generally, but not exclusively, a printing solution of the present invention replaces the ink solution of existing and commercially available printing devices, in particular within the printing cartridge.

Likewise, preferred printing methods include pad printing as those methods are known in the art, including but not limited to pad transfer printing. Pad printing is not as exact as digital printing but is a preferred method of printing for the present invention. Pad printing is known in the art for printing of images of the iris of the eye on contact lenses (see, U.S. Pat. Nos. 5,414,477, 5,302,978, and 4,668,240).

Ink jet printing is known in the art and can take various forms and associated structures as are discussed herein. Generally, ink jet printing refers to printing devices and methods that utilize highly precise printing methods and structures that allow for the production of high quality and precise structures. Generally, available inkjet printing devices and structures can be utilized with minimal modification, with the ink solutions normally present in the ink jet cartridge or reservoir is replaced with a solution that includes a polymerizable monomer and associated polymerization initiators as needed. The polymerizable monomer can be polymerized at will and at a rapid rate after being dispensed from the ink jet printing structure.

Three dimensional printing is based primarily, but not exclusively, on ink jet printing technologies. These methods and devices allow for the generation of one-off or multiple copies of a structure or structures. Generally, a polymerizable solutions is placed within the printing device and is dispensed under computer control and polymerized in repeated printing cycles or steps to generate a three dimensional structure. Examples of available and preferred 3D printing devices and related structures and cartridges include, but are not limited to: 3D Systems (www.3dsystems.com/default.asp) (Mar. 29-2011), ProJet™ 6000 Professional 3D Printer (http://printin3d.com/sites/printin3d.com/files/downloads/Projet_6000_brochure_USEN.pdf) (Mar. 29-2011); Stratasys, Inc. (http://www.stratasys.com/); Fortus 3D Production Systems—Fortus 900mc; Z Corporation (www.zcorp.com); Zprinter® 650 (http://www.zcorp.com/en/Products/3D-Printers/ZPrinter-650/spage.aspx) Vertical Resolution—90 to 100 microns (0.0035 to 0.004 in) Smallest Feature—100 microns (0.004 in); 3D Systems (http://www.3dsystems.com/default.asp); and Viper si2™ SLA® System

Piezo printing is a subtype of ink jet printing that is a preferable printing method of the present invention. Examples of available and preferred piezo printing devices and related structures and cartridges include, but are not limited to: MicroFab Technologies, Inc. (www.microfab.com) (Mar. 29-2011); Jetlab® 4xl, 4xl-A ((http://www.microfab.com/equipment/pdf/jetlab4xl_xla.pdf) (Mar. 29-2011); X-Y Accuracy/Repeatability—+/−25 microns/+/−5 microns (4xl-A); O.N.E Technologies (Mar. 29-2011); Material Deposition Systems (www.onelabs.com/matdep00.htm) (Mar. 29-2011), Resolution as low as 0.2 nanometer; Multi-Axis Printing Systems (Mar. 29-2011); FujiFilm USA|Dimatix, Inc. (Mar. 29-2011); Dimatix Materials Printer DMP-5000 Accuracy/Repeatability—+/−5 microns/+/−1 microns; Mimaki JF Series (Apr. 1-2011) Model JF1610 or JF 1631) (Apr. 1-2011), resolution up to 1200 by 1200 dpi.

Thermal printing is a subtype of ink jet printing that is a preferable printing method of the present invention. Examples of thermal printing devices and related structures and cartridges include, but are not limited to: Hewlett Packard (www.hp.com) (Apr. 1-2011); HP Designjet H45000 Printer Series.

Laser printing is a subtype of ink jet printing that is a preferable printing method of the present invention. Examples of laser printing devices and related structures and cartridges include, but are not limited to those known in the art such as Xerox Phaser 6010 laser printer or HP Color LaserJet Enterprise CP4025 Printer series—HP Color LaserJet Enterprise CP4025dn Printer (CC490A), or those later developed.

Optionally, a printing device, such as but not limited to an inkjet printing device, can include a rotating printer head. These types of printing structure can allow for enhanced printing on curved surfaces.

Another preferred printing method is MEMS printing is based on technologies that allow for the printing of integrated circuit boards, but are applicable to the production of very small structures that have functionality. Examples of structures having functionality made by MEMS printing include mechanical gears and other mechanical devices, lab on a chip structures for the performance of laboratory procedures including chemical reactions and diagnostic procedures.

Another preferred printing method is MEMS printing and is based on technologies that allow for the printing of integrated circuit boards, but are applicable to the production of very small structures that have functionality. Examples of structures having functionality made by MEMS printing include mechanical gears and other mechanical devices, lab on a chip structures for the performance of laboratory procedures including chemical reactions and diagnostic procedures.

### Printable Formulation

Printable formulations useful in the present invention for printing of layers or structures of the present invention using printing technologies as discussed herein and known in the art, particularly digital printing methods and technologies, can optionally include one or more drugs, any single drug compound or composition, or any combination of drug compounds or compositions. Printable formulations can be provided in water, monomer or solvents, preferably at a concentration between about 0% and greater than about 99.5% or between about 0.001% and about 99.5%, preferably between about 0.005% and about 90% or between about 1% and about 80%, and more preferably between about 10% and about 60% or between about 20% and about 40%. Printable formulations can also include particles or particulates, preferably at a concentration of between about 0% and about 15% or between about 0.001% and about 10%, preferably between about 0.005% and about 4% or between about 1% and about 3% to render a digitally printed formulation optionally with at least one drug. Examples of drugs include, but are not limited to, Timolol, Gentamycin and Nevanac. As discussed herein, the characteristics and compositions including printable formulations and other components include printable formulations that are or become part of an article of manufacture of the present invention, such as a lens, such as a contact lens, and also include compositions that include at least one printable formulations that can be used to make any article of manufacture of the present invention.

Printable formulations can include water, monomer, polymer or an appropriate solvent in order for the printable formulations to be suitable in the making of a digital print. An appropriate solvent is a solvent that is compatible with the creation of a print such as a digital print on or within a surface, such as on or within a polymer. For example, solvents appropriate for polymers used to make lenses, such as contact lenses, include, but are not limited to isopropanol, water, acetone or methanol, either alone or in combination and can include a monomer. Appropriate concentrations of solvents are between about 0% and greater than about 99.5% or between about 0.1% and about 99.5%, preferably between about 1% and about 90% or between about 10% and about 80%, and more preferably between about 20% and about 70% or between about 30% and about 60%. Different polymers, monomers and printable formulations have different tolerances and reactivity to different solvents. Thus, appropriate matches between solvent and polymer, monomer and printable formulations can be considered. For hydrogel polymers, adjustment in swelling ratios may be achieved with a variety of concentrations of solvents or crosslinkers.

A printable formulation can also include a monomer, polymer, homopolymer, heteropolymer, or copolymer. In a preferred aspect of this aspect of the present invention, a printable formulation includes a monomer that can be polymerized to form a polymer using polymerization methods appropriate for a given monomer, mixtures thereof, or polymers, or mixtures thereof. Monomers can also be used to decrease the viscosity of the printable formulation. Alternatively, the printable formulation can include a polymer such that the viscosity of the printable formulation is increased. Alternatively, the printable formulation can include polymer and monomer. Appropriate concentrations of monomers are between about 5% and greater than 99%, preferably between about 25% and about 75%, and more preferably between about 35% and about 60%. Appropriate concentrations of polymers are between about 0% and about 50%, preferably between about 5% and about 25%, and more preferably between about 10% and about 20%. When monomers and polymers are mixed, the total concentration of monomer and polymer are between about 10% and greater than 99%, preferably between about 25% and about 75% and more preferably between about 35% and about 65%.

The viscosity of a solution including a printable formulation can be as high as between about 500 centipoise and about 5,000 centipoise and is preferably between about 1 to about 200 centipoise or between about 10 and about 80 centipoise, preferably between about 20 and about 70 centipoise or between about 30 and about 60 centipoise or between about 1 and about 10 centipoise. Solutions having low viscosity tend to be “runny” when dispensed, and can allow different colors to merge and blend, resulting in an image with a more natural appearance. Such blending can be enhanced using a variety of methods, including sonication or vibration at appropriate duration and frequency to promote appropriate blending. Solutions having too low a viscosity can result in images that are too “runny” and thus have potentially undesirable characteristics, such as pooling of a printable formulation in a digitally encoded image or spreading of a printable formulation to an unintended location. Solutions having too high a viscosity may be easily dispensed using pad printing but are not suitable for other printing. Furthermore, solutions having high viscosity can tend to “bead” on a surface and not blend with the surrounding environment, including surrounding droplets or beads of printing formulation. Agents such as thickeners or diluents (including appropriate solvents) can be used to adjust the viscosity of the printable formulation.

Alternatively, one may use drug receiving layer that holds inkjetted digital droplets in its place until fixed. Another approach can be to use printable formulations that uses derivatized oligomer to be able to stop it from running by instant curing. Both of these approaches are discussed herein.

A printable formulation that includes at least one monomer can also include a polymerization initiator, so that once a printable formulation that includes at least one type of monomer is dispensed, the polymerization of the monomer in the printable formulation is initiated. The number, type and amount of initiator is a matter of choice depending on the type of monomer or monomers in the printable formulation. Appropriate initiators include, but are not limited to, UV initiators that initiate polymerization by UV irradiation, thermal initiators that initiate polymerization by thermal energy.

A printable formulation can also include a dispersant to allow uniform composition of formulation in a container. Dispersants are preferably provided at an appropriate concentration, such as between about 1% and about 10%.

A printable formulation can also include at least one anti-microbial agent or antiseptic agent to kill or reduce the number or multiplication microbial agents, reduce the number of microbial agents, or keep microbial agents from multiplying. Preferred anti-microbial agents include anti-bacterial agents, anti-fungal agents and disinfectants. Preferably, such anti-microbial agents, anti-bacterial agents, anti-fungal agents and disinfectants are provided at an appropriate concentration such as between about 0% and about 1%.

A printable formulation can also include at least one humectant such as 1,3-diozane-5,5-dimethanol (U.S. Pat. No. 5,389,132) at an appropriate concentration. Preferably, the range of concentration of a humectant is between about 0% and about 2%.

A printable formulation can also include at least one antioxidant agent or a low corrosion agent, such as alkylated hydroquinone, at an appropriate concentration, such as between about 0.1% and about 1% (U.S. Pat. No. 4,793,264). A PF can also include a non-kogating agent or non-kogating agent, such as 2-methyl-1,3-propanediol at an appropriate concentration, such as between about 0% and about 1%. A printable formulation can also include an evaporation retarding agent, such as, for example, diethylene glycerol or ethylene glycol at between about 0% and about 2% (U.S. Pat. No. 5,389,132).

A preferred printable formulation can have the following composition:

| Component | Percentage |
|---|---|
| Monomer | 0% to 99% |
| Drug or Encapsulated Drug | 0% to 25% |
| Initiator | 0.01% to 2% |
| Solvent | 0% to 80% |
| Binder or Bonding Agent | 0% to 10% |
| Thickener | 0% to 1% |
| Anti-kogating Agent | 0% to 1% |
| Humectant | 0% to 1% |
| Surfactant | 0% to 10% |
| Cross-linker | 0% to 1% |
| Dispersant | 0% to 10% |

### Modulation of Release of Drug

As previously discussed, the combination of the layers and components of the coating of the present invention serve to modulate the release of at least one drug from the coating.

A variety of physical and chemical forces influence the modulation of the release of a drug from a coating of the present invention. These include, but are not limited to diffusion characteristics of at least one layer of a coating of the present invention or the coating itself, capillary action characteristics of at least one layer of a coating of the present invention or the coating itself, mass action characteristics of at least one layer of a coating of the present invention or the coating itself, concentration gradient of a drug in at least one layer of a coating of the present invention or the coating itself, solubility of a drug characteristics of at least one layer in a coating of the present invention or the coating itself, temperature, molecular weight of a drug, size of a drug, encapsulation structures for a drug, thickness of at least one layer of a coating of the present invention or the coating itself, porosity of at least one layer of a coating of the present invention or the coating itself, the pore size of at least one layer of a coating of the present invention or the coating itself, the molecular exclusion size or characteristics of at least one layer of a coating of the present invention or the coating itself, the water content of at least one layer of the coating of the present invention or the coating itself, the concentration of a drug in at least one layer of a coating of the present invention or the coating itself, the concentration gradient of a drug in at least one layer of a coating of the present invention or the coating itself, and the packaging environment presented to the coating of the present invention.

In one aspect of the present invention, the at least one drug has sustained release over time. In another aspect of the present invention, the at least one drug has intermittent release over time. In yet another aspect of the present invention, more than one drug is released at a time.

### Contact Lens

In one preferred aspect of the present invention, the medical device having a coating being made includes a contact lens. Contact lenses that include a drug, on the surface of the contact lens or within the contact lens are known in the art. However, these contact lenses do not provide the structures of the present invention, such as the at least one coating that includes at least one drug reservoir layer that can include at least one drug, and at least one layer that can include structures, wherein the release of the at least one drug from the at least one coating layer is modulated by at least one layer of the coating of the present, either alone or in combination.

The choice of printing technologies used to make the various layers of the coating of the present invention, including the coating layer as a whole, is a choice for the artisan based on the state of the art and the teachings provided herein, as well as an evaluation of the various factors to consider when choosing a printing technology to produce a structure having desired chemical and physical properties, along with a consideration of the printing formation to be used.

A variety of materials are known in the art for making contact lenses and are useful in the present invention. Preferred materials include, but are not limited to, acrylics, silicones, polyvinylalcohols, and combinations thereof. These materials are provided on the surface of the contact lens to be modified using the methods of the present invention.

There are a variety of general types of contact lenses known in the art and are useful in the present invention. Preferred general types of contact lenses include, but are not limited to hybrid lenses, hydrophilic lenses and hydrophilic lenses. These types of contact lenses provide a surface of the contact lens to be modified using the methods of the present invention.

In addition, there are other general types of contact lenses known in the art and are useful in the present invention. These lenses include, but are not limited to spherical lenses, toric lenses, multifocal lenses, tinted lenses, corrective optical power lenses and lenses without corrective optical power. These types of contact lenses provide a surface of the contact lens to be modified using the methods of the present invention

There are a variety of methods used to make lenses that are useful in the present invention. Preferred methods of making, at least in part or in combination, contact lenses include, but are not limited to, lathing, cast molding, spin casting and ink jet printing. These contact lenses provide a surface of the contact lens to be modified using the methods of the present invention

Once a contact lens is manufactured, a variety of secondary or finishing operations can be utilized and are useful in the present invention. Preferred secondary or finishing operations include, but are not limited to edging, polishing, tinting, hydration, extraction, and sterilization. These secondary or finishing operations can optionally take place before or after the contact lens is modified by a method of the present invention, or both.

In one aspect of the present invention, the at least one drug in an at least one coating layer can be provided on the surface of a contact lens. In another aspect of the present invention, the at least one drug in at least one coating layer can be provided within a contact lens. In another aspect of the present invention, the at least one drug can be provided inside a contact lens without the structures in an at least one coating layer in combination with at least one drug in at least one coating layer on the surface of a lens. In yet another aspect of the present invention, the at least one coating layer with at least one drug can be provided both on the surface of the lens and inside the lens.

In some cases, drugs provided within the at least one coating can have optical properties that can interfere with the optical function of the contact lens, such as drugs having coloring or opaqueness. Preferred drugs for use in the present invention do not have such optical properties, but that need not be the case as drugs having such optical properties are useful in the present invention.

In another aspect of the present invention, the one or more coatings can optionally dispersed therein nanoparticles having a particles size less than about 50 nm, a nanoencapsulated ophthalmic drug from which the ophthalmic drug is able to diffuse into and migration through the contact lens and into the post-lens tear film or towards the eyelid when the contact lens is placed on the eye, the nanoparticles being disperse within the contact lens or on at least one surface of the contact lens in an amount such that the lens optionally remains substantially optically transparent (see, for example, U.S. Pat. No. 7,638,137B2 to Chauhan et al., issued Dec. 29, 2009).

In another aspect of the present invention, the one or more coatings can optionally dispersed therein nanoparticles having a particles size less than about 50 nm, a nanoencapsulated ophthalmic drug from which the ophthalmic drug is able to diffuse away from and migrate away from the contact lens and into the post-lens tear film or towards the eyelid when the contact lens is placed on the eye, the nanoparticles being disperse within the contact lens or on at least one surface of the contact lens in an amount such that the lens optionally remains substantially optically transparent (see, for example, U.S. Pat. No. 7,638,137B2 to Chauhan et al., issued Dec. 29, 2009).

In yet another aspect of the present invention, when the at least one drug is provided with or without a drug delivery compositions as described herein, the at least one drug as provided with or without a drug delivery compositions is substantially optically transparent. However, this need not be the case. In one aspect of the present invention, when the at least one drug as provided with or without a drug delivery composition is substantially optically transparent or is not substantially optically transparent, the optical characteristics of the at least one drug, or other structures of the at least one coating layer, can be masked with opaque material or tinting, such as color tinting as is known in the art.

### Packaging

An article of manufacture made by a method of the present invention can be provided in a variety for forms and packaging formats and solutions as present. Many of these packaging form and formats are established packaging formats, whereas others are unique to the present invention.

The article of manufacture made by a method of the present invention can be provided in a packaging in a dry state, preferably in a dehydrated state or a lyophilized state using methods know in the art. The article of manufacture made by a method of the present invention can also be provided in a packaging in a wet state, that is to say provided in an appropriate solution and, as appropriate, in a hydrated state.

The format of the packaging can be any as is appropriate. For example, the article of manufacture made by a method of the present invention can be provided in packaging that is appropriate and normal for the article of manufacture, such as vials, other containers such as boxes or plastic containers, or in vials. Vials and blister packaging are preferable, but not necessary, for example, for contact lenses.

The solution present, if any, in a packaging format, in particular for a wet state packaging format can include the at least one drug present in the at least one coating layer, a different drug that that provided in the coating layer, or a combination thereof.

In one instance, the concentration of the drug in a packaging solution is less than the concentration of the drug in the coating layer. In that case, it is likely that the drug in the coating layer may migrate from the coating layer into the packaging layer and eventually reach a steady state equilibrium state, but that not be the case.

In another instance, the concentration of the drug in a packaging solution is equal to the concentration of the drug in the coating layer. In that case, it is likely that the drug in the packaging solution will be in steady state with the drug in the coating layer, but that need not be the case.

In the alternative, the concentration of the drug in the packaging solution is greater than the concentration of the drug in the coating layer. In that case, it is likely that the drug in the packaging solution would migrate into the coating layer and eventually reach a steady state equilibrium state, but that need not be the case.

In yet another instance, a drug provided in the packaging layer that is not present in the coating layer may be present. In that case, it is likely that the drug in the packaging solution would migrate into the contact lens and eventually reaches a steady state equilibrium state, but that need not be the case.

### III Methods of Using Lenses Including a Medicament

The present invention includes method of treating or preventing a disease, disorder or condition or condition including: a) providing a subject in need of treatment of said disease, disorder or condition; and b) providing the subject the article of manufacture of the present invention, optionally made using the methods of the present invention, at a location appropriate for the treatment of said disease, disorder or condition; wherein the article of manufacture releases the one or more drugs in an amount sufficient to treat or prevent said disease, disorder or condition.

The article of manufacture of the present invention, its components and a compositions along with their desirable characteristics and selection criteria, how they are arranged and function together, and what criteria can be utilized to select and arrange them for a particular article of manufacture for a particular purpose, have been described herein. In addition, the methods of manufacture of the article of manufacture of the present invention, along with the manufacture of the coating layer and its various components, including but not limited to the drug reservoir layer, and the drug receiving layer, along with the printing formulations and printing technologies used to make them and the physical characteristics of the modulation of drug release therefrom, along with the criteria for selecting them for the manufacture of an article of manufacture for a particular purpose have also been described herein. The criteria for the selection of a drug, including for what purpose it is to be used for, its physical characteristics, its concentration, release characteristics and modulation thereof, have also been described herein.

An article of manufacture of the present invention, optionally made by a method of the present invention, tailored for the treatment or prevention of a particular disease, disorder or condition, and the drug has been selected and provided for in the article of manufacture such that the release characteristics have been evaluated based on the desired dose, regime, route of administration and locus of administration, and the pharmacological characteristics of the drug is provided. The drug has preferably been selected to match the disease, disorder or condition at hand, along with the locus at which it is released based on the criteria disclosed herein and provided by the state of the art.

A subject in need of treatment or prevention of a disease disorder or condition is also provided. The article of manufacture is then place on or within the subject at a desirable location using methods known in the art based on the locus at which the article of manufacture of the present invention is place (such as, but not limited, insertion on a surface, insertion, or implantation, inclusive of surgery if called for) such that the drug is released from the article of manufacture to treat or prevent a disease, disorder or condition. When the drug has been released over time, the article of manufacture can be removed from the subject, or in the alternative, removed from the subject. In the case of an article of manufacture of the present invention that has been placed on readily accessible locus of a subject, such as the skin or eye, the removal is readily performed. In the case of articles of manufacture of the present invention that have been implanted or inserted into a subject, the removal process is more complex and may require surgery. In some instances, removal of an article of manufacture of the present invention from a subject is not desirable due to the discomfort or risk associated with the removal. In that instance, the article of manufacture can remain in place.

### SPECIFIC ASPECTS OF THE PRESENT INVENTION

The specific aspects of the present invention section incorporates by reference the general aspects of the present invention provided above, along with all other sections and content of the present application.

### IV Packaged Medical Device Including a Medicament

Another aspect of the present invention includes a packaged medical device, including: a) at least one drug delivery contact lens, including: 1) at least one coating provided on at least one surface of the drug delivery contact lens; a) wherein the at least one coating includes at least one polymerized PEG monomer; b) further wherein the at least one coating includes at least one drug; b) at least one packaging solution, including: 1) the at least one of the at least one drug; c) at least one packaging, including: 1) the at least one drug delivery contact lens; and 2) the at least one packaging solution.

### Drug

A further aspect of the present invention includes wherein the at least one drug is provided in a pharmaceutically effective amount.

An additional aspect of the present invention includes wherein the at least one drug includes an antibiotic, an intraocular pressure reducing agent, a comfort enhancing agent, an anti-inflammatory agent, a penetration enhancer, a macular degeneration agent, or a combination thereof.

Another aspect of the present invention includes wherein the at least one drug includes gentamicin, tobramycin, erythromycin, polytrim, cirproflizacin, viamox, xymar, moxifloxacin, gatifolxacin or a combination thereof.

A further aspect of the present invention includes wherein the at least one drug includes timolol, alphagan, axopt, cosopt, lumigan, travatan, xalatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

An additional aspect of the present invention includes wherein the at least one drug includes sodium hyaluronate, hyaluronic acid (HA), cyclosporine, polyethylene glycol 400, hypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

Another aspect of the present invention includes wherein the at least one drug includes, perdforte, lotemax, fluromethlone, nevanac, acular, xibrom, or a combination thereof.

A further aspect of the present invention includes wherein the at least one drug includes, Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

An additional aspect of the present invention includes wherein the at least one drug includes EYLEA® (aflibercept).

Another aspect of the present invention includes wherein the at least one drug includes dexamethasone, pilocarpine nitrate, tropicamide, methyl prednisolone, flurbiprofen, penicillin ciprofloxacin, sulphacetaminde sodium, indomethacin, hydrocortisone, ndomethacin, iprofloxacin hydrochloride, insulin, indomethacin, ketorolac tromethamine, or a combination thereof.

A further aspect of the present invention includes wherein the at least one drug includes atropine.

An additional aspect of the present invention includes wherein the at least one drug includes HA, bimatoprost, or a combination thereof.

### 1600 Hyaluronic Acid (HA)

Another aspect of the present invention includes wherein the at least one drug includes hyaluronic acid (HA).

A further aspect of the present invention includes wherein the HA has a molecular weight between about 5 kDa and about 4,000 kDa.

An additional aspect of the present invention includes wherein the HA has a molecular weight between about 500 kDa and about 2,500 kDa

Another aspect of the present invention includes wherein the HA is not modified.

A further aspect of the present invention includes wherein the HA is modified.

An additional aspect of the present invention includes wherein the modified includes adding at least one functional group to the HA.

Another aspect of the present invention includes wherein the at least one function functional group includes methacrylate, acrylate, carboxymethyl, trimethylsilyl, thioethyl, butandiol-diglycidyl ether, divinyl sulfone, amidated HA, tyramine HA, hydrazide HA, thiolated HA, glycidyl methacrylate-HA, or a combination thereof.

A further aspect of the present invention includes wherein the HA is capable of binding water at between about 4 grams and about 5 grams of water per gram HA.

An additional aspect of the present invention includes wherein the HA has an intrinsic viscosity of between about 0.5 and about 3 m³/kg.

Another aspect of the present invention includes wherein the HA is steam sterilizable.

### Packaging Solution

A further aspect of the present invention includes wherein the at least one packaging solution includes an antibiotic, an intraocular pressure reducing agent, a comfort enhancing agent, an anti-inflammatory agent, a penetration enhancer, a macular degeneration agent, or a combination thereof.

An additional aspect of the present invention includes wherein the at least one packaging solution includes gentamicin, tobramycin, erythromycin, polytrim, cirproflizacin, viamox, xymar, or a combination thereof.

Another aspect of the present invention includes wherein the at least one packaging solution includes timolol, alphagan, axopt, cosopt, lumigan, travatan, xalatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

A further aspect of the present invention includes wherein the at least one packaging solution includes sodium hyaluronate, hyaluronic acid, cyclosporine, polyethylene glycol 400, hypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

An additional aspect of the present invention includes wherein the at least one packaging solution includes, Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

Another aspect of the present invention includes wherein the at least one packaging solution includes, perdforte, lotemax, fluromethlone, nevanac, acular, xibrom, or a combination thereof.

A further aspect of the present invention includes wherein the at least one packaging solution includes atropine.

An additional aspect of the present invention includes wherein the at least one drug includes HA, bimatoprost, or a combination thereof.

Another aspect of the present invention includes wherein the at least one packaging solution does not include a preservative.

### 1600 Hyaluronic Acid (HA)

A further aspect of the present invention includes wherein the at least one drug includes hyaluronic acid (HA).

An additional aspect of the present invention includes wherein the HA has a molecular weight between about 5 kDa and about 4,000 kDa.

Another aspect of the present invention includes wherein the HA has a molecular weight between about 500 kDa and about 2,500 kDa

A further aspect of the present invention includes wherein the HA is not modified.

An additional aspect of the present invention includes wherein the HA is modified.

Another aspect of the present invention includes wherein the modified includes adding at least one functional group to the HA.

A further aspect of the present invention includes wherein the at least one function functional group includes methacrylate, acrylate, carboxymethyl, trimethylsilyl, thioethyl, butandiol-diglycidyl ether, divinyl sulfone, amidated HA, tyramine HA, hydrazide HA, thiolated HA, glycidyl methacrylate-HA, or a combination thereof.

An additional aspect of the present invention includes wherein the HA is capable of binding water at between about 4 grams and about 5 grams of water per gram HA.

Another aspect of the present invention includes wherein the HA has an intrinsic viscosity of between about 0.5 and about 3.0 m³/kg.

A further aspect of the present invention includes wherein the HA is steam sterilizable.

### Packaging

An additional aspect of the present invention includes wherein the at least one packaging includes at least one blister pack.

Another aspect of the present invention includes wherein the at least one packaging includes at least one vial.

### Printing

A further aspect of the present invention includes wherein the at least one coating is made in whole or in part by at least one additive printing.

An additional aspect of the present invention includes wherein the at least one additive printing includes ink jet printing.

Another aspect of the present invention includes wherein the at least one additive printing includes digital printing, 3D printing, digital 3D printing, or a combination thereof.

A further aspect of the present invention includes wherein the at least one additive printing includes additive 3D printing.

### Coating

An additional aspect of the present invention includes wherein the at least one coating includes one or more layers.

Another aspect of the present invention includes wherein the at least one coating has a viscosity between about 10 and about 35cpscP.

A further aspect of the present invention includes wherein the at least one coating is made at least in part by polymerizing.

An additional aspect of the present invention includes wherein the polymerizing includes photopolymerizing.

Another aspect of the present invention includes wherein the photopolymerizing includes UV curing.

A further aspect of the present invention includes wherein the UV curing is for between about 1 and about 15 minutes.

An additional aspect of the present invention includes wherein the at least one coating is made using filtered materials.

Another aspect of the present invention includes wherein the filtered materials are made using a filter having a pore size between about 1 and about 10 microns.

A further aspect of the present invention includes wherein the at least one coating has at least one of the at least one drug dissolved within it.

An additional aspect of the present invention includes wherein the at least one coating has at least one of the at least one drug suspended within it.

Another aspect of the present invention includes wherein the at least one coating has the at least one drug loaded within it using microemulsion, micelles, encapsulation, nanoparticle, spray drying, in solvent, microspheres, liposomes, ethosomes, niosomes, biodegradable nanoparticles, temperature sensitive smart particles, or a combination thereof.

### Polymerized PEG Monomer

A further aspect of the present invention includes wherein the at least one polymerized PEG polymer is made using at least one polymerizable PEG monomer.

An additional aspect of the present invention includes wherein the at least one polymerizable PEG monomer includes at least one carbon-carbon double bond in its structure.

Another aspect of the present invention includes wherein the at least one polymerizable PEG monomer is polymerized through free radical polymerization.

A further aspect of the present invention includes wherein the at least one polymerizable PEG monomer is polymerized by free radical photopolymerization.

An additional aspect of the present invention includes wherein the at least one polymerizable PEG monomer is not biodegradable.

Another aspect of the present invention includes wherein the at least one polymerizable PEG monomer includes PEG-diacrylate, mono-PEG acrylate, PEG-diacrylamide, mono-PEG acrylamide, PEG-dimethacrylate, mono-PEG methacrylate, PEG-dimethacrylamide, mono-PEG methacrylamide, n-PEG-Acrylate, n-PEG-acrylamide, PEG diacetylene, PEG divinyl sulfone, or a combination thereof.

A further aspect of the present invention includes wherein the at least one polymerizable PEG monomer includes PEG-acrylate, PEG-acrylamide, PEG-methacrylate, PEG-methacrylamide, n-PEG-Acrylate, n-PEG-acrylamide, PEG acetylene, PEG vinyl sulfone, or a combination thereof.

An additional aspect of the present invention includes wherein the at least one polymerizable PEG polymer can absorb a drug from solution.

Another aspect of the present invention includes wherein the at least one polymerizable PEG monomer is present at a concentration of between about 2 and about 10% w/w.

A further aspect of the present invention includes wherein the at least one polymerizable PEG monomer is liquid at ambient temperature.

An additional aspect of the present invention includes wherein the at least one polymerizable PEG monomer is molecular weight of between about 200 Da to about 1,000 Da.

Another aspect of the present invention includes wherein when in use, HA from the drug delivery contact lens is released from about 1 hour to about 30 days.

A further aspect of the present invention includes wherein when in use, HA from the drug delivery contact lens is released from about 1 hour to about 7 days.

An additional aspect of the present invention includes wherein the pore size of the at least one coating is between about 1 and about 100 nm.

### Sterilization

Another aspect of the present invention includes wherein the packaged medical device, the at least one drug delivery contact lens, the at least one packaging solution, the at least one packaging, or a combination thereof, are sterilized, steam sterilized, or a combination thereof.

### V Drug Delivery Contact Lens

An aspect of the present invention includes A medical device, includes: a) at least one drug delivery contact lens, including: 1) at least one coating provided on at least one surface of the drug delivery contact lens; a) wherein the at least one coating includes at least one polymerized PEG monomer; b) further wherein the at least one coating includes at least one of the at least one drug; and 2) at least one drug.

### VI Method of Making a Drug Delivery Contact Lens

An aspect of the present invention includes a method of making a medical device, including: a) providing a contact lens; b) providing a printable solution including: 1) at least one polymerizable PEG monomer; 2) at least one drug; c) producing at least one coating on at least one surface of the contact lens using at least one additive printing d) where the contact lens is steam sterilized in its final packaging.

### VII Method of Using a Drug Delivery Contact Lens

An aspect of the present invention includes a method of using a medical device to treat at least one disease, disorder, or condition of the eye, including: a) providing a subject in need of treatment of a disease, disorder, or condition of the eye; b) providing at least one drug delivery contact lens of the present invention provided in a packaged medical device of the present invention; c) operably engaging the at least one drug delivery contact lens with at least one eye of the subject; wherein the at least one eye of the subject is treated for the at least one disease, disorder, or condition of the eye.

Another aspect of the present invention includes a method of using a medical device to treat at least one disease, disorder, or condition of the eye, including: a) providing a subject in need of treatment of a disease, disorder, or condition of the eye; b) providing at least one drug delivery contact lens of present invention; c) operably engaging the at least one drug delivery contact lens with at least one eye of the subject; wherein the at least one eye of the subject is treated for the at least one disease, disorder, or condition of the eye.

### VIII Bimatoprost and NVP Medical Device

An aspect of the present invention includes a medical device, including: at least one drug delivery contact lens, including: 1) at least one coating provided on at least one surface of the drug delivery contact lens; 2) bimatoprost; and 3) N-Vinylbyrrolidone (NVP); wherein the NVP modulates the release of the bimatoprost from the coating.

### Coating

Another aspect of the present invention includes wherein the at least one coating is made in whole or in part by additive printing.

A further aspect of the present invention includes wherein the at least one coating includes one or more layers.

### Printing

An additional aspect of the present invention includes wherein the at least one coating is made at least in part by at least one additive printing.

An aspect of the present invention includes wherein the at least one additive printing includes ink jet printing.

A further aspect of the present invention includes wherein the at least one additive printing includes digital printing, 3D printing, digital 3D printing, or a combination thereof.

An additional aspect of the present invention includes wherein the at least one additive printing includes additive printing, additive 3D printing, or a combination thereof.

### Additional Drugs

An aspect of the present invention includes wherein the at least one drug delivery contact lens further includes at least one additional drug.

Another aspect of the present invention includes wherein the at least one additional drug is provided in a pharmaceutically effective amount.

A further aspect of the present invention includes wherein the at least one additional drug further includes an antibiotic, an intraocular pressure reducing agent, a comfort enhancing agent, an anti-inflammatory agent, a penetration enhancer, a macular degeneration agent, or a combination thereof.

An aspect of the present invention includes wherein the at least one additional drug further includes dorzolamide, timolol, or a combination thereof.

Another aspect of the present invention includes wherein the at least one additional drug further comprises timolol, alphagan, axopt, cosopt, 68yprome, travatan, alatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

A further aspect of the present invention includes wherein the at least one additional drug further includes sodium hyaluronate, hyaluronic acid, cyclosporine, polyethylene glycol 400, 68ypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

An additional aspect of the present invention includes wherein the at least one additional drug further includes perdforte, lotemax, fluromethlone, nevanac, acular, xibrom, or a combination thereof.

An aspect of the present invention includes wherein the at least one additional drug further includes Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

Another aspect of the present invention includes wherein the at least one additional drug further includes Afliberset®.

A further aspect of the present invention includes wherein the at least one additional drug further includes dexamethasone, pilocarpine nitrate, tropicamide, methyl prednisolone, flurbiprofen, penicillin, ciprofloxacin, sulphacetaminde sodium, indomethacin, hydrocortisone, indomethacin, ciprofloxacin hydrochloride, insulin, indomethacin, ketorolac tromethamine, or a combination thereof.

An additional aspect of the present invention includes wherein the at least one additional drug further includes gentamicin, tobramycin, erythromycin, polytrim, cirproflizacin, viamox, xymar, or a combination thereof.

An aspect of the present invention includes wherein the at least one additional drug further includes atropine.

### IX. Contact Lens Soaking Solution

An aspect of the present invention includes a contact lens soaking solution, including: a) at least one drug, including one or more of: 1) at least one hyaluronic acid (HA); 2) at least one other drug; and b) at least one pharmaceutically acceptable carrier.

The loading solution includes but is not limited to the packaging solution of the present invention.

A soaking solution is used to soak a lens in order to maintain a desirable environment for a lens, such as but not limited to equilibrium or maintenance of levels of compounds in the lens. Such compounds can include but are not limited to drugs, including but not limited to HA, but other compounds can be included.

A soaking solution can also be used to deliver or remove one or more compounds to a lens in a soaking solution, such as but not limited to such as by mass action. Such compounds can include but are not limited to drugs, including but not limited to HA, but other compounds can be included.

### Loading Solution

Another aspect of the present invention includes wherein the soaking solution is a loading solution for at least one new contact lens.

A loading solution can be used to deliver or remove one or more compounds to a lens in a loading solution, such as but not limited to such as by mass action. Such compounds can include but are not limited to drugs, including but not limited to HA, but other compounds can be included. Generally, loading solutions are used to deliver one or more compounds to a lens that has a lower concentration of a compound, such as but not limited to one or more drugs, such as but not limited to HA.

Loading solutions are preferably used to deliver compounds to new lenses, but that need not be the case. Such new lenses can include compounds such as but not limited to drugs, including but not limited to HA. Such new lenses can include such compounds, but can also not have such compounds.

### Refreshing Solution

A further aspect of the present invention includes wherein the soaking solution is a refreshing solution for at least one used contact lens.

A refreshing solution can be used to deliver or remove one or more compounds to a lens in a loading solution, such as but not limited to such as by mass action. Such compounds can include but are not limited to drugs, including but not limited to HA, but other compounds can be included. Generally, refreshing solutions are used to deliver one or more compounds to a lens that has a lower concentration of a compound, such as but not limited to one or more drugs, such as but not limited to HA.

Loading solutions are preferably used to deliver compounds to used lenses, but that need not be the case. Such new lenses can include compounds such as but not limited to drugs, including but not limited to HA. Such used lenses can include such compounds, but can also not have such compounds.

Refreshing solution can be the same as the packaging solution, or as modified for pH, concentration of drug, bioactive agents such as but not limited to HA, trehalose, or at least one surfactant, at least one thermal stabilizer to change drug loading, modulate drug release, enhance product stability, or combinations thereof.

### Pharmaceutically Effective Amount

An additional aspect of the present invention includes wherein the at least one drug is provided in a pharmaceutically effective amount.

### Pharmaceutically Acceptable Carrier

An aspect of the present invention includes wherein the at least one pharmaceutically acceptable carrier is compatible with the eye.

The at least one pharmaceutically acceptable carrier can include at least one additional drug, such as but not limited to cyclosporine A for which bioactive agent like trehalose can mitigate dry eye syndrome.

Another aspect of the present invention includes wherein the at least one other drug includes an antibiotic, an intraocular pressure reducing agent, a comfort enhancing agent, an anti-inflammatory agent, a penetration enhancer, a macular degeneration agent, or a combination thereof. Examples of such agents include HA for comfort enhancement or trehalose for antiinflammation.

A further aspect of the present invention includes wherein the at least one other drug includes gentamicin, tobramycin, erythromycin, polytrim, cirproflizacin, viamox, xymar, moxifloxacin, gatifolxacin or a combination thereof.

An additional aspect of the present invention includes wherein the at least one other drug includes timolol, alphagan, axopt, cosopt, lumigan, travatan, xalatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

An aspect of the present invention includes wherein the at least one other drug includes sodium hyaluronate, cyclosporine, polyethylene glycol 400, hypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

Another aspect of the present invention includes wherein the at least one other drug includes, perdforte, lotemax, fluromethlone, nevanac, acular, xibrom, or a combination thereof.

A further aspect of the present invention includes wherein the at least one other drug includes, Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

An additional aspect of the present invention includes wherein the at least one other drug includes EYLEA® (aflibercept) or cycloporin A

An aspect of the present invention includes wherein the at least one other drug includes dexamethasone, pilocarpine nitrate, tropicamide, methyl prednisolone, flurbiprofen, penicillin ciprofloxacin, sulphacetaminde sodium, indomethacin, hydrocortisone, indomethacin, ciprofloxacin hydrochloride, insulin, indomethacin, ketorolac tromethamine, or a combination thereof.

An aspect of the present invention includes wherein the at least one other drug includes atropine.

Another aspect of the present invention includes wherein the at least one other drug includes bimatoprost,

### Hyaluronic Acid

A further aspect of the present invention includes wherein the at least one HA has a molecular weight between about 5 kDa and about 4,000 kDa.

An additional aspect of the present invention includes wherein the at least one HA has a molecular weight between about 500 kDa and about 2,500 kDa

An aspect of the present invention includes wherein the at least one HA is not modified.

Another aspect of the present invention includes wherein the at least one HA is modified.

A further aspect of the present invention includes wherein the modified includes adding at least one functional group to the at least one HA.

An additional aspect of the present invention includes wherein the at least one function functional group includes methacrylate, acrylate, carboxymethyl, trimethylsilyl, thioethyl, butandiol-diglycidyl ether, divinyl sulfone, amidated HA, tyramine HA, hydrazide HA, thiolated HA, glycidyl methacrylate-HA, or a combination thereof.

An aspect of the present invention includes wherein the at least one HA is capable of binding water at between about 4 grams and about 5 grams of water per milligram HA.

Another aspect of the present invention includes wherein the at least one HA has an intrinsic viscosity of between about 0.5 and about 3 m³/kg.

A further aspect of the present invention includes wherein the at least one HA is steam sterilizable.

An additional aspect of the present invention includes wherein the HA is modified.

An aspect of the present invention includes wherein the modified includes adding at least one functional group to the HA.

Another aspect of the present invention includes wherein when in use, the at least one HA from the drug delivery contact lens is released from about 1 hour to about 30 days.

A further aspect of the present invention includes wherein when in use, the at least one HA from the drug delivery contact lens is released from about 1 hour to about 7 days.

### X. Method of Loading Contact Lenses

An aspect of the present invention includes a method of loading a new contact lens with at least one drug, including: a) providing at least one new contact lens that includes PEG; b) providing at least one loading solution of the present invention; c) allowing the at least one drug in the at least one loading solution to load into the at least one new contact lens.

Another aspect of the present invention includes wherein the at least one new contact lens includes said drug delivery contact lens of the present invention.

A further aspect of the present invention includes wherein the at least one new contact lens includes HA.

An additional aspect of the present invention includes wherein the at least one new contact lens does not include HA.

An aspect of the present invention includes a method of refreshing a used contact lens with at least one drug, including: a) providing at least one used contact lens that includes PEG; b) providing a refreshing solution of the present invention; and c) allowing the at least one drug in the at least one refreshing solution to load into the at least one used contact lens.

Another aspect of the present invention includes wherein the at least one used contact lens includes a drug delivery contact lens of the present invention.

A further aspect of the present invention includes wherein the at least one used contact lens comprises HA.

An additional aspect of the present invention includes wherein the at least one used contact lens does not include HA.

### XI. Loaded and Refreshed Contact Lenses

An aspect of the present invention includes a loaded contact lens, made by a method of the present invention.

Another aspect of the present invention includes wherein when in use, the loaded contact lens can elute HA a) at a molecular weight of between about 10,000 Dalton to about 3,000,000 Dalton; b) over a time period of more than about 8 hours to about 7 days; or c) combinations thereof.

A further aspect of the present invention includes wherein said HA has a range of molecular weight of between about 25,000 Da to about 1,500,000 Da, from between about 50,000 Da to about 1,000,000 Da, or combinations thereof.

An aspect of the present invention includes a refreshed contact lens, made by a method of the present invention.

Another aspect of the present invention includes wherein when in use, the loaded contact lens can elute HA a) at a molecular weight of between about 10,000 Dalton to about 3,000,000 Dalton; b) over a time period of more than about 8 hours to about 7 days; or c) combinations thereof.

A further aspect of the present invention includes wherein said HA has a range of molecular weight of between about 25,000 Da to about 1,500,000 Da, from between about 50,000 Da to about 1,000,000 Da, or combinations thereof.

An aspect of the present invention includes a refreshed contact lens, made by a method of the present invention.

### XII. Method of Using Loaded and Refreshed Contact Lenses

An aspect of the present invention includes a method of using the loaded contact lens of the present invention, including: a) providing a subject in need of the loaded contact lens; b) providing at least one loaded contact lens of the present invention; and c) operably engaging the at least one loaded contact lens with at least one eye of the subject.

Another aspect of the present invention includes wherein when in use, the loaded contact lens can elute HA a) at a molecular weight of between about 10,000 Dalton to about 3,000,000 Dalton; b) over a time period of more than about 8 hours to about 7 days; or c) combinations thereof.

A further aspect of the present invention includes wherein said HA has a range of molecular weight of between about 25,000 Da to about 1,500,000 Da, from between about 50,000 Da to about 1,000,000 Da, or combinations thereof.

Another aspect of the present invention includes wherein the at least one eye of the subject is treated for at least one disease, disorder, or condition of the at least one eye.

An aspect of the present invention includes a method of using the refreshed contact lens of present invention, including: a) providing a subject in need of the refreshed contact lens; b) providing at least one refreshed contact lens of the present invention; and c) operably engaging the at least one refreshed contact lens with at least one eye of the subject.

Another aspect of the present invention includes wherein when in use, the loaded contact lens can elute HA a) at a molecular weight of between about 10,000 Dalton to about 3,000,000 Dalton; b) over a time period of more than about 8 hours to about 7 days; or c) combinations thereof.

A further aspect of the present invention includes wherein said HA has a range of molecular weight of between about 25,000 Da to about 1,500,000 Da, from between about 50,000 Da to about 1,000,000 Da, or combinations thereof.

Another aspect of the present invention includes wherein the at least one eye of the subject is treated for at least one disease, disorder, or condition of the at least one eye.

### EXAMPLES

### Example 1: Contact Lens Having Slow Release of Glaucoma Treating Drug

### A. General Characterization of Contact Lens Having Slow Release Bimatoprost

Bimatoprost release from contact lens can be modulated by changing the percent content of N-Vinylpyrrolidone (NVP) in the coating.

This control is believed to be achieved because NVP and Bimatoprost hydrogen bond, as such:

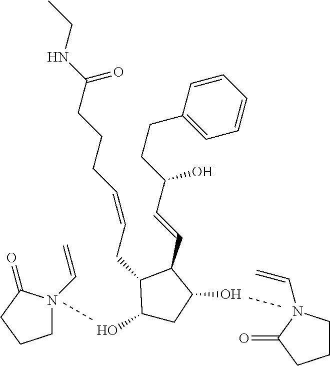

Bimatoprost hydroxyl groups (hydrogen donator groups) can hydrogen bond with NVP's nitrogen atom (hydrogen acceptor groups), which aids in the lens retaining Bimatoprost via the coating.

Therefore, the release rate can be modulated based on the amount of NVP, the more NVP is in the coating, the more potential hydrogen bond acceptor groups are available for Bimatoprost to interact with. This will slow the release of Bimatoprost.

Contrarily, the less NVP in the coating, the faster that Bimatoprost will release from the contact lens.

Changing the percent content of NVP in the MediPrint coating has another effect as well: the higher percent of NVP in the coating, the higher initial assay value of Bimatoprost, and vice versa. This is due to the same interaction of hydrogen bonding between Bimatoprost and NVP.

### B. Method of Making Contact Lens Having Slow Release of Bimatoprost Problem:

Ophthalmic drugs have faced issues in drug delivery, such as low bioavailability and patient non-compliance. This is due to drugs generally being administered via eyedrops. Eyedrops present the above issues because much of the payload (the drop) is generally lost due to tear drainage, which leads to about 5% of the drug making it into the eye, or 5% bioavailability. Furthermore, due to the inconvenience of eyedrops, patients often forget to administer the treatment, with about 50% compliance rate for single eyedrops. Contact lenses have been proposed as a drug delivery system for ophthalmic drugs, in order to improve bioavailability and patient compliance. With a contact lens, the drug's residence time on the tear film is around 30 minutes, which is improved from the eyedrops' 2 minutes. Bioavailability is therefore increased to as much as 50%. However, without some modification, a contact lens is unlikely to sustain release of an ophthalmic drug for more than a few hours.

### Solution:

The present invention provides a procedure to coat a hydrogel contact lens with a drug coating, via inkjet printing. This coating can modulate the release of drug, such that it will sustain release of the drug for one week or longer. For example, N-Vinylpyrrolidone (NVP) can be used to modulate the release of Bimatoprost (BMT). The more NVP is in the coating, the slower BMT will release from the contact lens, and vice versa. This control is believed to be achieved because NVP and Bimatoprost hydrogen bond, as such (though applicants do not wish to be limited to, and are not limited to, a particular mechanism):

Bimatoprost hydroxyl groups (hydrogen donator groups) can hydrogen bond with NVP's nitrogen atom (hydrogen acceptor groups), which aids in the lens retaining Bimatoprost via the coating.

Therefore, the release rate can be modulated based on the amount of NVP in the coating.

LL-BMT lenses are produced by printing about 1 to 5 mg of UV coating containing various methacrylate monomers and bimatoprost (BMT) on the surface of a dry lens. One of these monomers is called N-Vinyl Pyrrolidinone, and it may play a principal role in controlling the release rate of BMT from the lens. This is believed to be due to NVP hydrogen-bonding to BMT via the latter molecule's hydroxyl groups, which aid in BMT retention on the lens.

It is possible that the release rate can be modulated based on the amount of NVP, the more NVP is in the coating, the more potential hydrogen bond acceptor groups are available for Bimatoprost to interact with. This will slow the release of Bimatoprost. Contrarily, the less NVP in the coating, the faster that Bimatoprost will release from the contact lens.

Once the dry lens has been printed on, the lens is hydrated to remove unreacted monomers. The hydrated lens is then sterilized and allowed to equilibrate in a packaging solution (PS), which is made with a known concentration of Bimatoprost, preferably about the same concentration as in or on the lens, though more or less are included as part of the present invention. The drug release profile of the finished drug coated lenses for LL-BMT is measured by extraction of bimatoprost in 2 mL simulated tear fluid (STF). This is called the in-vitro study.

The BMT drug release at various time intervals is sampled by taking and replacing 2 mL of STF every 24 hours up to 192 hours, and the concentration of BMT at all time intervals is determined by LCMS. There are two sections of the drug release profile, the burst release (0-24 hours) and the sustained release section (24-192 hours). The sustained release section of the drug release profile of LL-BMT1 show first order drug release kinetics in in-vitro experiments. The effect of modulating NVP levels in the coating on the release kinetics was then explored, in order to determine if the release rate could be controlled by modifying the percentage of NVP in the UV-coating of LL-BMT1.

### Study Materials and Samples:

LL-BMT1 lenses were made via the following process: On a dry hydrogel contact lenses around 1 to 5 mg of a BMT-containing methacrylate monomer mixture was inkjet printed onto the contact lens, and cured to polymerize via UV light.

There are several steps to creating the BMT-containing methacrylate monomer mixture, the BMT-coating.

First, a derivatized oligomer was prepared by polymerizing a collection of monomers while in the presence of a free radical scavenger “quencher” molecule to prevent full polymerization. Preferable monomers polymerize through a chain reaction, and therefore contain a C═C double bond in their structure. More preferably, such monomers that are hydrophilic and can absorb water in their structure when polymerized can be used in the production of derivatized oligomer. Such preferable monomers and their concentrations for oligomerization are included in TABLE 1.

TABLE 1
Preferable Monomers and Concentrations for Oligomerization

| Monomer | Concentration (% w/w) |
|---|---|
| HEMA (hydroxyethyl methacrylate) | 20-45% |
| DMA (dimethylacrylamide) | 3-10% |
| GMA (glycerol monomethacrylate) | 3-10% |
| PVA (polyvinylalcohol) | 3-10% |
| NVP (N-Vinyl Pyrrolidinone) | 6-15% |
| MAA (methacrylic acid) | 2-10% |
| MM (methyl methacrylate) | 2-10% |
| Silicone | 0.5-5% |
| Siloxane | 0.5-5% |

The listed monomers are preferable, but not limited to only the monomers in Table 1.

The total percentage of monomers was then about 40-70% w/w of the whole oligomer mixture. Preferable quencher molecules and their concentrations are included in Table 2.

TABLE 2
Preferable Quenchers and Concentrations for Oligomerization

| Quencher | Concentration (% w/w) |
|---|---|
| 2-mercaptoethanol | 0.5-3% |
| α-tocopherol | 0.5-3% |
| Lipoic Acid | 0.5-3% |

The listed quenchers are preferable, but not limited to only the quenchers in TABLE 2.

The total percentage of quenchers was then about 0.5-9% w/w of the whole oligomer mixture. Oligomerization is triggered via a free-radical initiator. Preferable initiators are solids or liquids that can dissolve into/are miscible with organic solvents. More preferable initiators are triggered via heat, and these initiators and their concentrations are included in TABLE 3:

TABLE 3
Preferable Initiators and Concentrations for Oligomerization

| Initiator | Concentration (% w/w) |
|---|---|
| 2,2′-azobis(methylbutyronitrile) | 0.1-0.5% |
| Azobisisobutyronitrile | 0.1-0.5% |
| 1,1′-Azodicyclohexanecarbonitrile | 0.1-0.5% |

The listed initiators are preferable, but not limited to only the initiators in TABLE 3.

The total percentage of initiators was then about 0.1-1.5% w/w of the whole oligomer mixture. The chosen hydrophilic monomers, quencher, and initiator were added together in a solvent or combination of solvents and stirred until a clear solution was formed. Preferable solvents and their concentrations are included in TABLE 4:

TABLE 4
Preferable Solvents and Concentrations for Oligomerization

| Solvent | Concentration (% w/w) |
|---|---|
| 1-methoxy-2-propanol | 30-60% |
| Propylene glycol monomethyl ether acetate | 5-15% |
| 1-ethoxy-2-propanol | 30-60% |
| 1-hexanol | 20-40% |
| 1-octanol | 20-40% |

The listed solvents are preferable, but not limited to only the initiators in TABLE 4.

The total percentage of solvent was then about 25-60% w/w. The mixture is stirred, and heated to the initiator's activation temperature, and then kept at 70-90° C. for a controlled period of time from 30 minutes to two hours, until the solution is sufficiently viscous. The oligomer's viscosity is then confirmed to be 2000-3000 cP, and is remade if the viscosity is outside this range.

Once the oligomer is prepared, it is incorporated into a mixture containing a collection of monomers. Preferable monomers polymerize through a chain reaction, and therefore contain a C═C double bond in their structure. More preferably, such monomers that are liquid at ambient temperature, and that are hydrophilic and can absorb water in their structure when polymerized can be used in the production of Masterbatch. One of these monomers is N-Vinyl Pyrrolidinone (NVP). A specific amount of NVP from 10-30% w/w is included in the Masterbatch. Other Preferable monomers and concentrations are included in TABLE 5.

TABLE 5
Preferable Monomers and Concentrations
for Masterbatch Preparation

| Monomer | Concentration (% w/w) |
|---|---|
| HEMA (hydroxyethyl methacrylate) | 20-60% |
| DMA (dimethylacrylamide) | 2-10% |
| GMA (glycerol monomethacrylate) | 5-20% |
| PVA (polyvinylalcohol) | 5-20% |
| MAA (methacrylic acid) | 2-10% |
| MM (methyl methacrylate) | 2-10% |
| Low molecule Siloxane (TRIS) | 010% |
| Macromic Siloxane (PDMS) | 0-10% |

The listed monomers are preferable, but not limited to only the monomers in TABLE 5.

The monomers and oligomer are mixed together at about a 80:20 w/w ratio. The mixture was magnetically stirred until a clear solution formed. This monomer/oligomer mixture is called the Masterbatch.

A BMT-microemulsion was prepared. This microemulsion was prepared by mixing together an aqueous phase containing water at 5-20% w/w, and a surfactant or surfactants. Preferable surfactants have an HLB value of 8-18 such as to form an oil-in-water microemulsion. More preferable surfactants are nonionic surfactants with the stated HLB value. Such surfactants are included in TABLE 6:

TABLE 6
Preferable Surfactants and Concentrations
for Microemulsion Preparation

| Surfactant | Concentration (% w/w) |
|---|---|
| Polysorbate 80 | 30-60% |
| Polysorbate 20 | 30-60% |
| Polysorbate 60 | 30-60% |
| Poloxamer 407 | 5-20% |
| 1,3-propandiol | 30-60% |
| surfynol CT 121 | 10-30% |
| Polyethylene glycol 400 | 40-70% |

The listed surfactants are preferable, but not limited to only the surfactants in TABLE 6.

The total percentage of surfactant(s) is 20-60% w/w in the microemulsion. The solution was mixed with the drug and an oil until the solution was clear—this signifies that the microemulsion has formed. An oil can be any substance that is not miscible with water without a surfactant. More preferable oils are fatty acids, and examples are included in TABLE 7.

TABLE 7
Preferable Oils and Concentrations for Microemulsion Preparation

| Oil | Concentration (% w/w) |
|---|---|
| Glycerin | 2-10% |
| Octanoic Acid | 2-10% |
| Stearic Acid | 2-10% |

The listed oils are preferable, but not limited to only the oils in TABLE 7.

The drug is Bimatoprost, mixed in a concentration of 5-20% w/w. The three components (aqueous phase, drug, oil) are mixed together with the drug (BMT) until a homogenous microemulsion is formed.

Hyaluronic Acid (HA) was mixed with a liquid surfactants like Tween-20, Tween-80 or Polyethylene Glycol 400 by triturating together in a roughly 70:30 w/w ratio until a white amorphous paste is formed. This combination prevents HA from clumping together during the formulation of BMT-coating. The combination is called Ink-HA.

Masterbatch, BMT-microemulsion, and Ink-HA were mixed together to make BMT-coating, in a ratio that makes Bimatoprost 0.5-5% w/w in the solution, and HA at 0.25-2.5% w/w. Masterbatch makes up the rest of the formulation. The coating is homogenized until a uniform coating is produced, and the viscosity of the coating is then measured, and confirmed to be 5-40 cp. ty. Then, an initiator or initiators are added to the solution. Preferable initiators are solid or liquid, and can dissolve into/are miscible with the UV-coating. More preferable initiators are UV-light activated, and these initiators and their concentrations are included in TABLE 8.

TABLE 8
Preferable Initiators for Preparation of UV-ME-Coating

| Initiator | Concentration (% w/w) |
|---|---|
| Irgacure 184 | 2-10% |
| Irgacure 500 | 2-10% |
| Irgacure 1300 | 2-10% |
| Darocur 1173 | 2-10% |
| Irgacure 907 | 2-10% |

The listed initiators are preferable, but not limited to only the initiators in TABLE 8.

The coating is then loaded into the inkjet printer, and 1-5 mg of this coating is printed onto dry hydrogel contact lenses via nozzles in the inkjet printer Xaar XJ-500 printhead, in an inkjet printer setup. The printed-on-contact lenses are then cured under a UV-emitting lamp at specific wavelengths from 210-400 nm, in order to polymerize the coating via a chain reaction. These lenses are cured for 1-60 minutes, so as not to let the drug (BMT) or HA possibly degrade. These printed-on dry contact lenses are then hydrated in a 0.5% w/w NaHCO₃ solution, and steam sterilized in a phosphate buffered packaging solution containing BMT in a concentration of 0.003-0.010% w/w. Seven lots of LL-BMT lenses, with varying % of NVP from 10-30% w/w in the Masterbatch, were prepared in this fashion.

The seven lots of LL-BMT lenses were tested in the in-vitro study to determine their drug release profile, and subsequently determine if there was any trend in drug release kinetics. The lots tested are shown in TABLE 9:

TABLE 9
Lot Number of LL-BMT Lenses and BMT concentrations
in packaging solution (PS)

| Lot Number | NVP % (w/w) |
|---|---|
| 190610BMMEHALW-50 | 30% |
| 190610BMMEHALW-60 | |
| 190619BMMEHALW-50 | |
| 190619BMMEHALW-60 | |
| 2000117BMMEHALW-J | 10% |
| 2000117BMMEHALW-K | 15% |
| 2000117BMMEHALW-L | 20% |

Three lenses from each of the 30% NVP lots manufactured for each time point were used to measure the drug release profile. Nine lenses from each lot manufactured with 10%, 15%, or 20% NVP were used for the measurement of the drug release profile.

Method used for the Four Lots Produced with 30% NVP

As mentioned above, three lenses were used from each sublot. STF samples from each timepoint were taken as shown in TABLE 10:

TABLE 10
In-vitro Flux Samples prepared for
30% NVP LL-BMT Lenses Produced:

| Code Name | Sample Name | Number of Lens | Time Point (h) |
|---|---|---|---|
| 1-1 | LL-BMT | 3^{a} | 1 |
| 1-3 | LL-BMT | 3^{a} | 3 |
| 1-6 | LL-BMT | 3^{a} | 6 |
| 1-12 | LL-BMT | 3^{a} | 12 |
| 1-24 | LL-BMT | 3^{b} | 24 |
| 1-48 | LL-BMT | 3^{b} | 48 |
| 1-72 | LL-BMT | 3^{b} | 72 |
| 1-96 | LL-BMT | 3^{b} | 96 |
| 1-120 | LL-BMT | 3^{b} | 120 |
| 1-144 | LL-BMT | 3^{b} | 144 |
| 1-168 | LL-BMT | 3^{b} | 168 |
| 1-192 | LL-BMT | 3^{b} | 192 |

^{a}A set of three lenses were used for each of these time points. At 1 hour, 3 hours, 6 hours, and 12 hours, 100 μL of STF was withdrawn and replaced with 100 μL fresh STF. The removed aliquot was diluted by a factor of 10. This sampling scheme was for the burst release section.
^{b}One set of same three lenses were used for these time points. At 24 hours, 48 hrs, 72 hrs, 96 hrs, 120 hrs, 144 hrs, 168 hrs, 2.0 mL of STF were withdrawn and transferred into glass containers from each of the lens and 2.0 mL of fresh STF was added to vials containing lenses for 48 hours, 72 hours, 96 hrs, 120 hrs, 144 hrs, and 168 hrs time points. This sampling scheme was used for the sustained release section.

After each sample was taken, the STF containing BMT was stored in a labeled 2 mL Eppendorf tube in ambient conditions. There were three tubes per lot per timepoint. For lot 190619BMMEHALW-50 and lot 190610BMMEHALW-60, all three STF samples per timepoint were tested by LCMS analysis. For lot 190619BMMEHALW-60 and lot 190610BMMEHALW-50, each tube had 335 μL of STF removed and pooled into a new tube, so that there would be one sample of 1 mL STF per timepoint. The samples were tested by LCMS analysis of BMT concentration.

Method used for the Lots Produced with 10-20% NVP

As mentioned above, nine lenses were used from each sublot. STF samples from each timepoint were taken as shown in TABLE 11:

TABLE 11
In-vitro Flux Samples prepared for LL-BMT Lenses:

| Code Name | Sample Name | Number of Lens | Time Point (h) |
|---|---|---|---|
| 1-1 | LL-BMT | 3^{a} | 1 |
| 1-6 | LL-BMT | 3^{a} | 6 |
| 1-24 | LL-BMT | 3^{b} | 24 |
| 1-48 | LL-BMT | 3^{b} | 48 |
| 1-72 | LL-BMT | 3^{b} | 72 |
| 1-96 | LL-BMT | 3^{b} | 96 |
| 1-120 | LL-BMT | 3^{b} | 120 |
| 1-144 | LL-BMT | 3^{b} | 144 |
| 1-168 | LL-BMT | 3^{b} | 168 |
| 1-192 | LL-BMT | 3^{b} | 192 |

^{a}One set of three lenses is used for this individual time point. 2.0 mL of STF was added to the three lenses at time 0, and at the timepoint specified, 2.0 mL of STF was withdrawn. The lens was not returned to the study after this withdrawal. This sampling scheme was used for the burst release section.
^{b}Same one set of three lens was used for the rest of the time points in the study. At 24 hours, 48 hrs, 72 hrs, 96 hrs, 120 hrs, 144 hrs, 168 hrs, 192 hrs, and 216 hrs time points, 2.0 mL of STF were withdrawn and transferred into glass containers from each of the lens and 2.0 mL of fresh STF was added to vials containing lenses for 48 hours, 72 hours, 96 hrs, 120 hrs, 144 hrs, and 168 hrs time points. This sampling scheme was used for the sustained release section.

After each sample was taken, the STF containing BMT was stored in a labeled 3 mL glass vial at 5° C. There were three vials per sublot per timepoint. The samples were then tested by LCMS for analysis of BMT concentration.

### Results

### 30% NVP Lots

The drug profile dataset (referred to as BMT Release by Timepoint) and the natural logarithm of each datapoint is displayed in TABLE 12 and TABLE 13 respectively.

TABLE 12
BMT Release by Timepoint - 30% NVP Lots⁴
BMT Release by Timepoint (ng)

| Time (h) | 190619-50 | 190619-60 | 190610-50 | 190610-60 |
|---|---|---|---|---|
| 1 | 12369 | 22655 | 13889 | 16748 |
| 3 | 360 | 2993 | 7269 | −1178^{a} |
| 6 | 1774 | 508 | −1714^{a} | 234 |
| 12 | 2760 | 3722 | 11118 | 986 |
| 24 | 5334 | 2723 | −7339^{a} | 9134 |
| 48 | 4374 | 8867 | 4793 | 5207 |
| 72 | 1249 | 1866 | 1133 | 1199 |
| 96 | 742 | 652 | 497 | 466 |
| 120 | 281 | 226 | 141 | 160 |
| 144 | 73.5 | 81.7 | 45.3 | 48.2 |
| 168 | 19.5 | 29.7 | 6.5 | 6.0 |
| 192 | 12.9 | 19.8 | 10.1 | 4.0 |

^{a}Possible sample preparation error and was not used in plot for the analysis

The BMT released per timepoint was used to determine if a trend existed in the release profile. This was further accomplished by taking the natural logarithm of each data point (when possible) and graphing the natural logarithm of each release point in a semilog plot as a function of time in hours.

TABLE 13
BMT Natural Logarithmic Release by Timepoint - 30% NVP Lots
BMT Release by Timepoint (ng)

| Time (h) | 190619-50 | 190619-60 | 190610-50 | 190610-60 |
|---|---|---|---|---|
| 1 | 9.42 | 10.03 | 9.54 | 9.73 |
| 3 | 5.89 | 8.00 | 8.89 | — |
| 6 | 7.48 | 6.23 | — | 5.46 |
| 12 | 7.92 | 8.22 | 9.32 | 6.89 |
| 24 | 8.58 | 7.91 | — | 9.12 |
| 48 | 8.38 | 9.09 | 8.47 | 8.56 |
| 72 | 7.13 | 7.53 | 7.03 | 7.09 |
| 96 | 6.61 | 6.48 | 6.21 | 6.14 |
| 120 | 5.64 | 5.42 | 4.95 | 5.08 |
| 144 | 4.30 | 4.40 | 3.81 | 3.88 |
| 168 | 2.97 | 3.39 | 1.87 | 1.79 |
| 192 | 2.56 | 2.99 | 2.31 | 1.39 |

The data in TABLE 13 was then plotted, first as a whole dataset, then only from 24 hours to 192 hours. The combined plots are shown below in FIG. 1.

Based on the full timescale, there is an initial phase of increased drug release to reach a maximum concentration within 24 hours and a subsequent phase of slow drug release for each of the four lots studied. There is no trend for kinetics based on the full drug release profile.

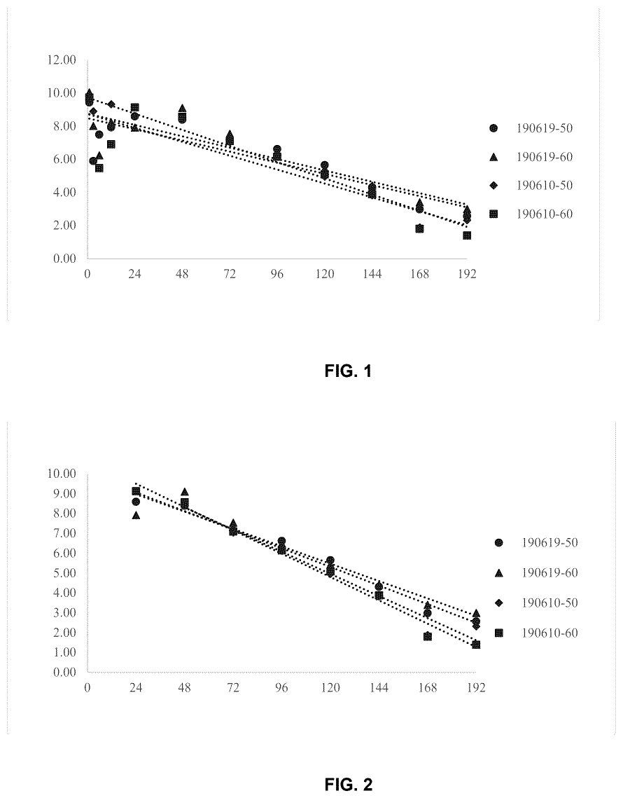

The sustained release portion of the drug profile was examined (24-192 hours) and the release was found to be first order kinetics. This is shown in FIG. 2:

TABLE 14 contains the rate constants and R² values for each trendline.

TABLE 14
Rate Constants and R² Values for 30% NVP Lens Lots

| Lot | k (rate constant) | R² |
|---|---|---|
| 190619BMMEHALW-50 | 0.039 | 0.9797 |
| 190619BMMEHALW-60 | 0.0364 | 0.9305 |
| 190610BMMEHALW-50 | 0.0464 | 0.9641 |
| 190610BMMEHALW-60 | 0.0489 | 0.9861 |

Because the R² values of each trendline are above 0.9, it may be concluded that the 30% NVP lenses follow first-order release kinetics, with a defined rate constant. The average rate constant of the 30% NVP lenses is 0.0427±0.0059, 13.82%.

### 10-20% NVP Lots

The drug release profile data for the 10-20% NVP Lots, as well as the natural logarithm of each datapoint, are shown in TABLE 15 and TABLE 16, respectively.

TABLE 15
BMT Release by Timepoint - 10-20% NVP Lots - 10-20% NVP

| | Time | Lot 200117- | Lot 200117- | Lot 200117- |
|---|---|---|---|---|
| | (hrs) | J - 10% NVP | K- 15% NVP | L- 20% NVP |
| BMT | 1 | 15888 | 15768 | 18768 |
| Release per | 6 | 15866 | 16672 | 13246 |
| Timepoint | 24 | 16670 | 17544 | 17502 |
| (ng/Timepoint) | 48 | 3712 | 4156 | 4026 |
| | 72 | 890 | 904 | 918 |
| | 96 | 220 | 200 | 194 |
| | 120 | 49.6 | 77.4 | 52.2 |
| | 144 | 11.8 | 17.4 | 12.2 |
| | 168 | 1.52 | 4.4 | 9.64 |
| | 192 | 3.86 | 4.02 | 2.26 |

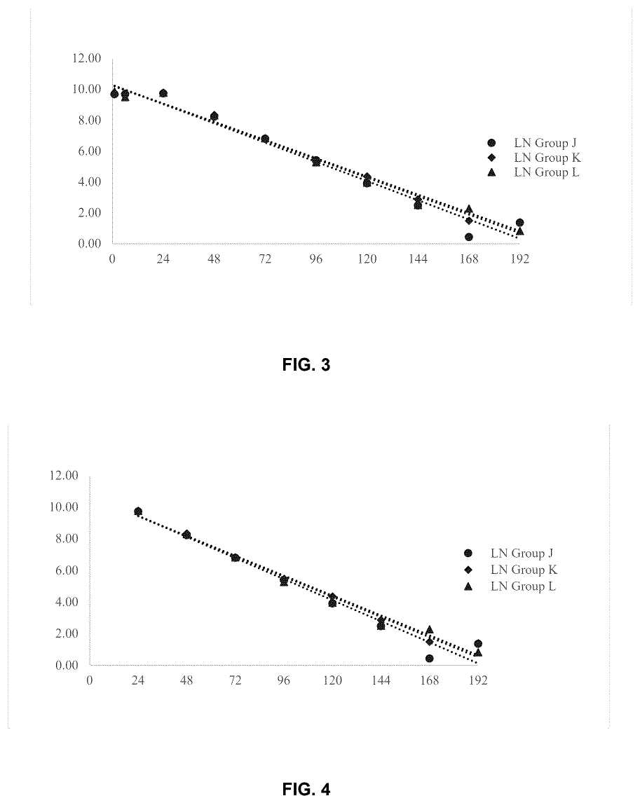

As performed before, the natural logarithms of each datapoint were plotted in a semi-log plot as a function of time. The BMT concentration at various time intervals plotted are shown in TABLE 16, and the plots are in FIG. 3 and FIG. 4.

TABLE 16
BMT Natural Logarithmic Release
by Timepoint - 10-20% NVP Lots

| | Time | Lot 200117- | Lot 200117- | Lot 200117- |
|---|---|---|---|---|
| | (hrs) | J - 10% NVP | K- 15% NVP | L- 20% NVP |
| Natural | 1 | 9.67 | 9.67 | 9.84 |
| Log of | 6 | 9.67 | 9.72 | 9.49 |
| BMT | 24 | 9.72 | 9.77 | 9.77 |
| Release | 48 | 8.22 | 8.33 | 8.30 |
| | 72 | 6.79 | 6.81 | 6.82 |
| | 96 | 5.39 | 5.30 | 5.27 |
| | 120 | 3.90 | 4.35 | 3.96 |
| | 144 | 2.47 | 2.86 | 2.50 |
| | 168 | 0.42 | 1.48 | 2.27 |
| | 192 | 1.35 | 1.39 | 0.82 |

As performed in the 30% NVP lots, the full timescale was plotted, and a plot was fitted to the data. The sustained release graphs again showed a linear trend with rate constant k, given by the slope of each plot, and therefore the presence of first-order kinetics from 24 hours onwards is confirmed for the various lots studied. The rate constants and R² values are below in Table 17:

TABLE 17
Rate Constants and R² Values for 10-20% NVP Lens Lots

| Lot | k (rate constant) | R² |
|---|---|---|
| 200117BMMEHALW - | 0.0556 | 0.9637 |
| Group J (10% NVP) | | |
| 200117BMMEHALW - | 0.0524 | 0.9835 |
| Group K (15% NVP) | | |
| 200117BMMEHALW - | 0.0531 | 0.9851 |
| Group L (20% NVP) | | |

The average rate constant is 0.0537±0.0017, 3.17%. Of the three lots, group K (15% NVP) showed the lowest rate constant, at 0.0524. The rate constant of K and L are very similar (1.34% difference), which can be explained by experimental variability such as error in timepoint sampling, or lens coating weight affecting release rate (a lens with more coating will contain more NVP, which lowers the rate constant, and vice versa).

The results of this study show that there exists clear first order kinetics in the sustained drug release phase of LL-BMT lenses, and that the rate constant is inversely related to the percentage of NVP in the coating—that is to say, a higher percentage of NVP in coating will have a lower rate constant and slower release of BMT, and a lower percentage of NVP in coating will have a higher release of BMT. This is shown when comparing the average rate constants of 30% NVP lenses and 10-20% NVP lenses; 0.0427±0.0059 in 30%, compared to 0.0537±0.0017 in 10-20%. The lower rate constant was connected to the higher % of NVP, and the higher rate constant was connected to the lower % of NVP. It can then be reasonably concluded that the release rate of BMT can be controlled by adjusting the level of NVP in the coating.

### C. Method of Using Contact Lens Having Slow Release of Bimatoprost

The LL-BMT lenses with 15% NVP in the coating and 26 μg/lens were tested in a clinical trial to evaluate the efficacy of LL-BMT1 in lowering IOP in primary-open angle glaucoma patients. 28 patients were enrolled in the study. 14 patients were enrolled in the test group of LL-BMT1, and the other 14 were enrolled in Timolol 0.5% b.i.d. The primary endpoint of the study was to evaluate the IOP drop from baseline in patients with POAG or OHT for up to 21 days, in both groups. Secondary objectives included evaluation of safety and tolerability of LL-BMT1 for up to 21 days. Key inclusion criteria for the trial included: 1. Male or female subject of at least 18 years of diagnosed with Primary Open-Angle Glaucoma (POAG) or Ocular Hypertension (OHT) in both eyes 2. At least one eye (called the study eye) must have an untreated IOP between 22 mmHg and 34 mmHg (both inclusive). (For eyes receiving IOP treatment, untreated IOP will be assessed following a required washout period) 3. No adverse reactions to nonmedicated contact lenses during the 2-week acclimation screening. Key exclusion criteria included: Glaucoma or optic neuropathy due to anything other than POAG or OHT, an IOP>34 mmHg in either eye at the screening visit or baseline Visit, or any severe and uncontrolled comorbid medical conditions or ocular condition which, in the opinion of the investigator, may either put the subject at risk because of participation in the study or may influence the results of the study.

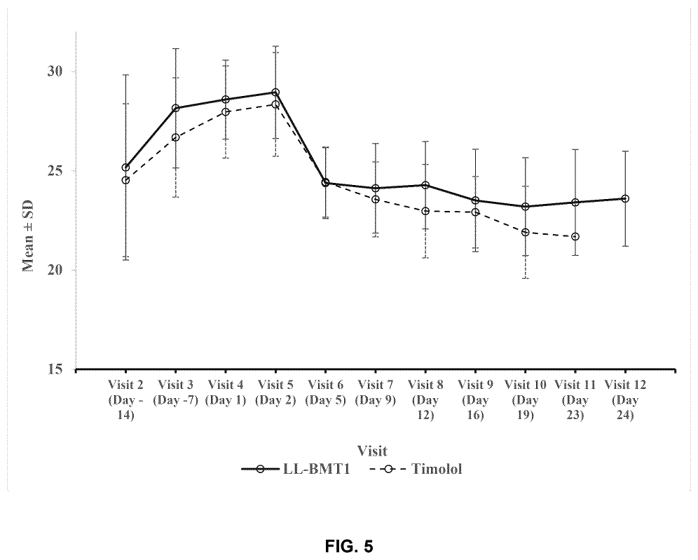

14 patients in each study arm were recruited, as per the inclusion criteria. In summary, LL-BMT1 was well tolerated, with two treatment emergent adverse events (TEAEs). In both eyes, a mean IOP drop of −5.45 mmHg was observed in patients wearing LL-BMT1, after 3 weeks. A mean IOP drop of −6.66 mm Hg was observed in patients using 0.5% Timolol. All measurements were taken at 8 AM, when the lens was inserted or the eyedrop was administered. LL-BMT1 was found to be effective in lowering IOP for the use in treatment of POAG. See FIG. 5.

A large drop in IOP was observed after the first lens insertion. At weeks 2 and 3, a smaller drop in IOP was observed after respective lens insertions.

The IOP was held at a relatively constant level in both LL-BMT1 and 0.5% Timolol throughout the study, after the large initial drop. This suggests the NVP in the coating is sustaining the release of BMT to be at a therapeutic level throughout the wear period of LL-BMT1.

A second clinical trial, with the LL-BMT lenses with 15% NVP in the coating and 32 μg/lens were tested in a clinical trial. The dose was set at 32 μg BMT to determine the effect of raising the dose on IOP drop. 28 patients were enrolled in the study. 14 patients were enrolled in the test group of LL-BMT1, and the other 14 were enrolled in bimatoprost 0.01% q.d. as a control. The primary endpoint of the study was to evaluate the IOP drop from baseline in patients with POAG or OHT for up to 21 days, in both groups. Secondary objectives included evaluation of safety and tolerability of LL-BMT1 for up to 21 days. Key inclusion criteria for the trial included: 1. Male or female subject of at least 18 years of diagnosed with Primary Open-Angle Glaucoma (POAG) or Ocular Hypertension (OHT) in both eyes 2. At least one eye (called the study eye) must have an untreated IOP between 22 mmHg and 34 mmHg (both inclusive). (For eyes receiving IOP treatment, untreated IOP will be assessed following a required washout period) 3. No adverse reactions to nonmedicated contact lenses during the 2-week acclimation screening. Key exclusion criteria included: Glaucoma or optic neuropathy due to anything other than POAG or OHT, an IOP>34 mmHg in either eye at the screening visit or baseline Visit, or any severe and uncontrolled comorbid medical conditions or ocular condition which, in the opinion of the investigator, may either put the subject at risk because of participation in the study or may influence the results of the study.

14 patients in each study arm were recruited, as per the inclusion criteria. In summary, LL-BMT1 was well tolerated. There were two TEAEs in the LL-BMT1 group, and one patient was discontinued as a result. In both eyes, a mean IOP drop of −8.2 mmHg was observed in patients wearing LL-BMT1, after 3 weeks. A mean IOP drop of −7.8 mm Hg was observed in patients using 0.5% Timolol. All measurements were taken at 8 AM, when the lens was inserted or the eyedrop was administered.

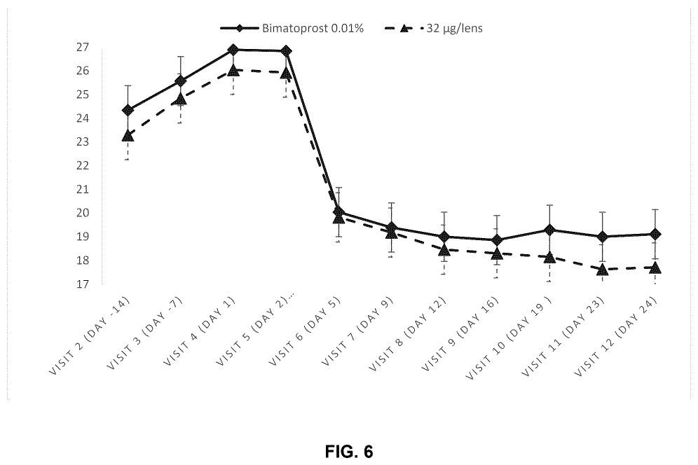

LL-BMT1 was found to be effective in lowering IOP for the use in treatment of POAG, and equivalent to bimatoprost 0.01% eyedrops as a result. See FIG. 6.

A large drop in IOP was observed after the first lens insertion, as seen before. The 32 μg BMT lens continued to lower IOP at a slower rate, whereas in the bimatoprost 0.01% arm, the IOP drop stayed constant.

### Example 2: Contact Lens Having Slow Release of Comfort Enhancing Agent

### A. Characterization of Contact Lens Having Slow-Release Hyaluronic Acid Problem:

Contact lenses, especially extended-wear contact lenses, can be very uncomfortable to wear for a variety of reasons. 51% of those who discontinue wearing contact lenses cite reasons such as dryness or protein accretion on the lens surface. Protein accumulation on the lens surface, deposited from the tear fluid during wear, can cause a variety of issues, such as inflammatory issues and discomfort. Dryness also causes discomfort during wear. Therefore, preventing these issues by inhibiting protein deposits on the lens and preventing the lens from inducing dryness in the eye is of great importance for an extended-wear contact lens.

Hyaluronic Acid (HA) has been used to aid comfort for the eye Hyaluronic acid is a naturally occurring glycosaminoglycan polymer found in the extracellular matrix of the body. Its structure is shown below, being Hyaluronic Acid shown as the repeating dimer unit that makes up HA.

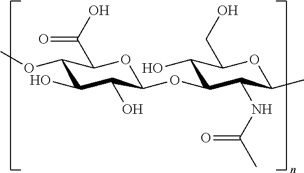

Hyaluronic acid may range from a molecular weight of 5 kDa up to weights of 4000 kDa, and may be produced for pharmaceutical use by bacteria such as Bacillus or streptococcus. HA absorbs high amounts of water, and may act as a lubricating agent. For these reasons, HA is used to aid comfort while wearing a contact lens or to relieve the symptoms of dry eye. Such purposes have been researched before. Zhang et al in 2021, in their paper “Hyaluronic acid in ocular drug delivery” explored several applications for HA, which include dry eye disease treatment, corneal wound healing, and as a comfort agent while wearing contact lens. Singh et al in 2015, in their paper “A hyaluronic acid-binding contact lens with enhanced water retention” modified a contact lens with an HA-binding peptide and formed a thin layer of HA on the surface of the lens when soaked in an HA-containing solution. This increased water retention of the lens. Thus, when HA is combined with contact lenses, HA can form a thin film on the surface of the contact lens that increases tear burst time, and inhibits protein deposits from forming on the lens surface. This thin film of HA may ultimately aid in contact lens comfort.

Additionally, it has been reported that HA released into the eye may treat symptoms of dry eye disease. Hynnekliev et al. in 2022, in their paper “Hyaluronic acid in the treatment of dry eye disease” found different doses of HA eyedrops from 0.1% to 0.4% all improved symptoms of dry eye disease. This is of great interest for daily or extended-wear contact lenses, to increase the comfort of wear and reduce the feelings of dryness during the entire contact lens wear period.

HA of high molecular weight (>1 MDa) is specifically hypothesized to be more helpful, as it has an increased residence time on the eye surface relative to lower molecular weight HA, and may absorb more water than lower molecular weight HA.

Thus, a desirable HA drug delivery system would include the following components:
1. Continuous delivery of HA, preferably of high-molecular weight H A
2. Sustained release of HA for a longer period of time from 8 hrs. to 7 days or more
3. A thin film of HA on the surface of contact lens that improves wettability as well as reduces friction associated with blinking
4. Provide stability to tear film formed between contact lens and cornea
5. Presence of HA on the surface of contact lens that prevents protein deposit.

Earlier attempts by others to incorporate HA with the lens have been attempted. One approach was to soak lenses in HA solution for release during wear, as explored by Chang et al. in 2021, in their paper “Applications of Hyaluronic Acid in Ophthalmology and Contact Lenses”. It was found that soaking the lens could hold HA, but most of the HA released rapidly, and not in a sustained manner. Thus, soaking an unmodified lens cannot serve its purpose for extended wear. In another approach, such as employed by Weeks et al. in 2013 in their paper “Physical entrapment of hyaluronic acid during synthesis results in extended release from model hydrogel and silicone hydrogel contact lens materials”, HA has been incorporated as a part of the pre-polymer mix for contact lenses. By combining HA in this fashion, HA may not have released as it may have been trapped in relatively thicker contact lens film cured over a longer period of time, 15 mins or longer. Such absence of thin film coating on surface of lens may not inhibit deposits of protein onto the lens surface. Finally, HA has been grafted onto the lens via covalent bonds, as performed by Korogiannaki et al. in 2019 in their paper “Impact of a Hyaluronic Acid-Grafted Layer on the Surface Properties of Model Silicone Hydrogel Contact Lenses”, but this approach has an unwieldy manufacturing process, nor does it release HA into the eye. Different approaches have involved modification of the HA molecule. While the unmodified molecule is found in the body, it may be modified by adding functional groups to the hydroxyl moieties on the structure, in order to improve wettability or to better incorporate into a contact lens. One example of a modification to HA is adding methacrylate groups to the molecule. Weeks et al. in 2012 in their paper “Photocrosslinkable hyaluronic acid as an internal wetting agent in model conventional and silicone hydrogel contact lenses” explored the photopolymerization of methacrylated HA into a hydrogel contact lens, and found that the HA-laden lenses were more hydrophilic and reduced protein adsorption. However, this approach did not allow for HA to release effectively, as most of it was polymerized in the lens. Additionally, modification of HA into methacrylated HA introduces an extra manufacturing step and therefore a lengthened manufacturing process.

Inkjet printing onto the surface of a lens has been utilized as a tactic to incorporate HA with the lens, as taught by Doshi in 2019 (U.S. Pat. No. 11,510,869). However, there are several issues with this approach:
1. HA (of any molecular weight) is difficult to inkjet print due to low solubility in the ink, so HA tends to form undissolved particles as a suspension in the ink.
2. HA tends to clump during ink formulation, which clogs inkjet printing nozzles.
   a. Spray dried HA coagulates even more than regular HA, so spray drying HA does not solve the issue.
3. Use of nanoparticles of HA and probe sonicated HA may lead to faster release of particles.
4. HA ink must be filtered through at least 2-micron filter prior to printing to prevent clogging of nozzles of inkjet printer, which filters HA out of the ink.
5. Thus after filtration—only small amounts of HA may be printed on the lens.

Inkjet printing provides only a small amount of HA onto the lens, and the printed HA is entrapped between the polymerized coating. This entrapped HA may hydrogen bond with free HA in the packaging solution, but the entrapped HA cannot be released due to the coating's small pore sizes. Thus, only one aspect of the ideal HA lens is formed, the HA thin film. This is not sufficient for comfort enhancement of the lens.

The present invention overcomes these limitations, and provides related benefits as well. To overcome these limitations, a polyethylene glycol polymerizable monomer is included in the coating formulation. The PEG monomer, when polymerized, forms a PEG hydrogel on the lens surface. This hydrogel, after soaked in an HA-containing packaging solution, absorbs HA, for release into the eye when worn. Importantly, this allows for the absorption and release of high molecular weight HA, which is preferable to low molecular weight for comfort enhancement. Therefore, both aspects of the ideal HA lens are formed: entrapped HA that facilitates a thin film of HA, and a sustained release of HA from an HA-loaded PEG hydrogel on the lens surface, for the application of comfort in an extended wear lens. Inclusion of the PEG monomer will create a large enough pore size in the hydrogel on lens of surface to absorb HA from surrounding solution. This larger pore size allows for the absorption and subsequent release of high molecular weight HA. The size of pores depends on the PEG monomer used. Smaller PEG monomers (M_{w}<1000 Da), when crosslinked, will produce smaller pore sizes than larger PEG monomers (M_{w}>1000 Da). Thus PEG monomers can be used to modulate drug release rate to some extent. Thus giving longer release duration.

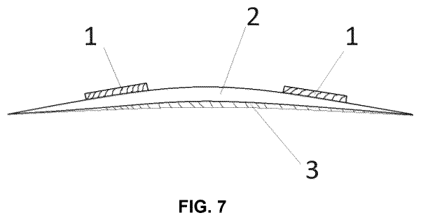

The present invention provides an HA formulation to achieve the desired properties:
1. Formation of film of thickness less than 1 micron to 150 microns made from hydrogen bonding to entrap HA on coated contact lenses and HA that modulates the release of HA.
2. Formation of a PEG hydrogel on surface of contact lens through photopolymerization and loading of the said hydrogel with HA of low and high molecular weight thereafter for the sustained release of HA for 8 hrs to more than 7 days. For such a lens it is also possible to reload HA from soaking solution, after desired use of a day or a week, to extend reuse of the same lens. The coated lens, with HA, is shown in FIG. 7:

### B. Method of Making a Contact Lens Having Slow Release of Hyaluronic Acid

Presented is a method to combine HA with a hydrogel contact lens, via inkjet printing. HA can then prevent protein deposits and dryness while the lens is worn. Therefore, HA can solve the principal obstacles around daily or extended contact lens wear by making the lens comfortable through the entire wear period. Such lens can also be used in combination with continuous release of many ocular drugs used for treating ocular diseases such as glaucoma, dry eye, allergy, infection, cataract etc., for one day, one week or more.

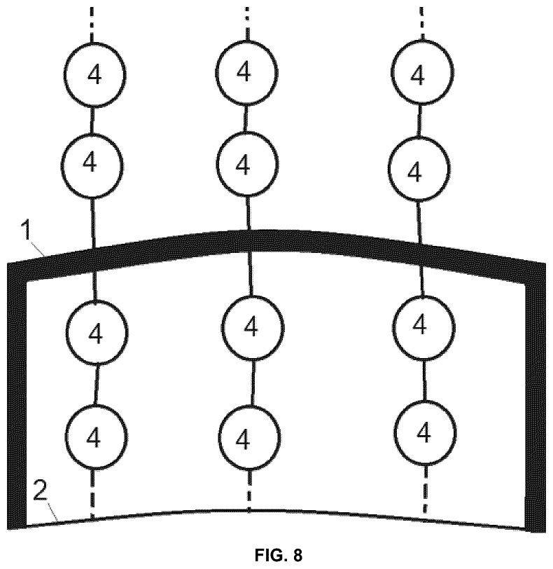

Examples: Hyaluronic Acid was inkjet printed onto the contact lens, by incorporating Hyaluronic Acid into a monomer solution, printing a controlled amount onto a dry contact lens surface, and polymerizing the mixture together. This physically entraps HA, as such in FIG. 8:

The HA and polymer are not covalently bonded to each other, but the HA molecules are physically trapped in the matrix. This is achieved by polymerizing for a time of about 1-15 minutes. This faster cure time promotes random crosslinking, which is less likely to occur when curing for 40 minutes or longer. The crosslinking then traps HA in the matrix. Both entrapped HA may hydrogen bond with other HA molecules, which helps to create the film of HA on the lens surface.

In addition to physically entrapping the HA, HA is loaded from the surrounding solution into the polymer matrix. This is due to the addition of a polyethylene glycol (PEG) monomer to the pre-polymer mixture. Polyethylene glycol monomers are known to form a hydrogel (PEG hydrogel), and when incorporated into the coating pre-polymer mix, may form this hydrogel after photopolymerization. PEG hydrogels have many uses in drug delivery. Wang et al. in 2023 in their paper “Poly Ethylene Glycol (PEG)-Based Hydrogels for Drug Delivery in Cancer Therapy: A Comprehensive Review” discussed the use of PEG hydrogels for cancer therapeutic delivery, and the hydrogels are highly biocompatible. Thus, forming a PEG hydrogel on the contact lens for the delivery of HA was logical for this purpose. The PEG hydrogel can be loaded with HA when soaked in an HA-containing packaging solution. Such loading may be further aided by temperature and high pressure of steam sterilization.

Furthermore, the pore size is relevant for drug delivery rate. Andrade del Omo et al. in 2022 in their paper “Sustained Drug Release from Biopolymer-Based Hydrogels and Hydrogel Coatings” explored different mechanisms for drug release from hydrogels, and found that pore size may determine the drug release rate. Different pore sizes may be created based on the PEG monomer(s) chosen for polymerization. Lower molecular weight PEG monomers will create smaller pore sizes, and larger molecular weight monomers will create larger pore sizes. Lee et al. in 2010 in their paper “Development of Macroporous Poly(ethylene glycol) Hydrogel Arrays Within Microfluidic Channels” (Biomacromolecules, 11(12), 3316-3324. https://doi.org/10.1021/bm100792y) showed that PEG-diacrylate hydrogels from monomers of 575-20000 Da in size formed pore sizes of 0.1-10 nm. It can be concluded that lower molecular weight PEG monomers will produce smaller pore sizes, and vice versa. Furthermore, using multi-armed PEG monomers will create more dense hydrogel networks via increased crosslinking. Finally, the concentration of PEG used in the coating will directly correlate with a more dense or less dense hydrogel network. In the coating, it can be estimated that the pore size ranges from 1-100 nm, because the PEG concentration is low. Different PEG monomers and the different hydrogel networks that may be formed are shown below, being different PEG Monomers. 1: PEG Dimethacrylate. 2: 4-PEG-Acrylate. There are four distinct arms of PEG. 3. PEG Diacrylate 4. PEG Divinyl sulfone

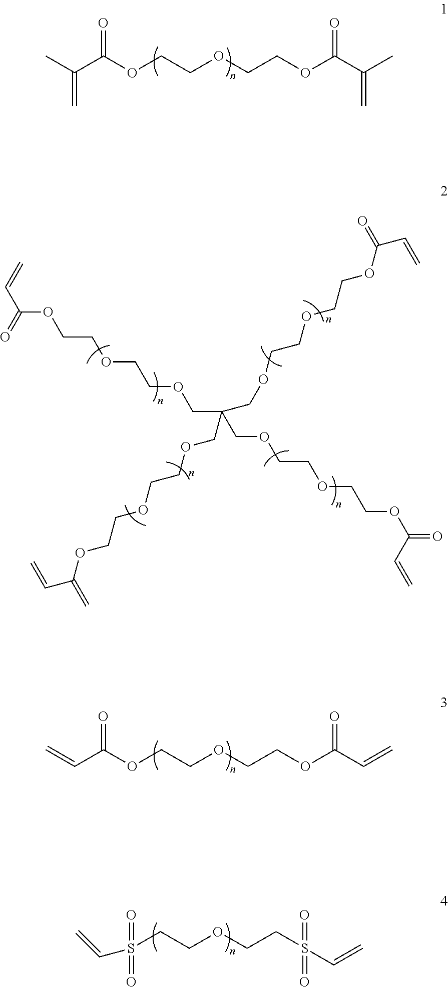

Potential hydrogel networks are depicted below, being formed by: 1. Hydrogel mesh formed by a low molecular weight PEG monomer (<1000 Da). 2. Hydrogel mesh formed by a high molecular weight PEG monomer (>1000 Da). 3. Hydrogel mesh formed by a combination of multiarm-PEG and other PEG monomers. The space between molecules are pores where HA is absorbed.

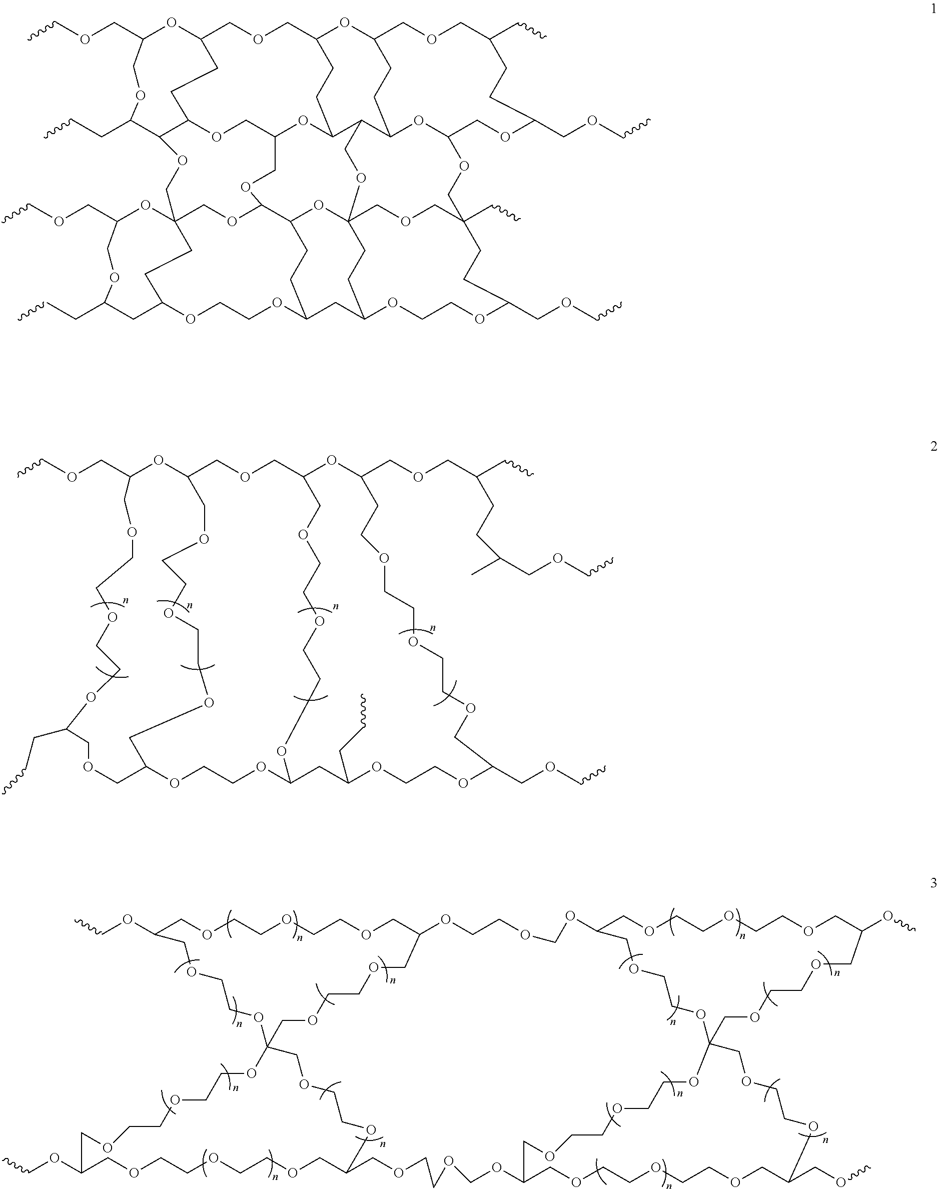

Dry hydrogel BioMedics™ contact were obtained from Cooper Vision, and 2 mg of a methacrylate monomer mixture was inkjet printed onto the contact lens, and cured to polymerize via UV light. There are several steps to creating the methacrylate monomer mixture, the UV-coating.

First, a derivatized oligomer was prepared by polymerizing a collection of monomers while in the presence of a free radical scavenger “quencher” molecule to prevent full polymerization. Preferable monomers polymerize through a chain reaction, and therefore contain a C═C double bond in their structure. Preferably, such monomers that are hydrophilic and can absorb water in their structure when polymerized can be used in the production of derivatized oligomer. Such preferable monomers and their concentrations for oligomerization are included in TABLE 18.

TABLE 18
Preferable Monomers and Concentrations for Oligomerization

| Monomer | Concentration (% w/w) |
|---|---|
| HEMA (hydroxyethyl methacrylate) | 20-45% |
| DMA (dimethylacrylamide) | 3-10% |
| GMA (glycerol monomethacrylate) | 3-10% |
| PVA (polyvinylalcohol) | 3-10% |
| NVP (N-Vinyl Pyrrolidinone) | 6-15% |
| MAA (methacrylic acid) | 2-10% |
| MM (methyl methacrylate) | 2-10% |
| TRIS (low molecular Siloxane | 0.5-5% |
| PDMS (macromeric Siloxane) | 0.5-5% |

The listed monomers are preferable, but not limited to only the monomers in TABLE 18.

The total percentage of monomers was then about 40-70% w/w of the whole oligomer mixture. Preferable quencher molecules and their concentrations are included in TABLE 19.

TABLE 19
Preferable Quenchers and Concentrations for Oligomerization

| Quencher | Concentration (% w/w) |
|---|---|
| 2-mercaptoethanol | 0.5-3% |
| α-tocopherol | 0.5-3% |
| Lipoic Acid | 0.5-3% |

The listed quenchers are preferable, but not limited to only the quenchers in TABLE 19.

The total percentage of quenchers was then about 0.5-9% w/w of the whole oligomer mixture. Oligomerization is triggered via a free-radical initiator. Preferable initiators are solids or liquids that can dissolve into/are miscible with organic solvents. Preferable initiators are triggered via heat and not UV-light, to prevent premature activation, and these initiators and their concentrations are included in TABLE 20:

TABLE 3
Preferable Initiators and Concentrations for Oligomerization

| Initiator | Concentration (% w/w) |
|---|---|
| 2,2′-azobis(methylbutyronitrile) | 0.1-0.5% |
| Azobisisobutyronitrile | 0.1-0.5% |
| 1,1′-Azodicyclohexanecarbonitrile | 0.1-0.5% |

The listed initiators are preferable, but not limited to only the initiators in TABLE 20.

The total percentage of initiators was then about 0.1-1.5% w/w of the whole oligomer mixture. The chosen hydrophilic monomers, quencher, and initiator were added together in a solvent or combination of solvents and stirred until a clear solution was formed. Preferable solvents and their concentrations are included in TABLE 21:

TABLE 21
Preferable Solvents and Concentrations for Oligomerization

| Solvent | Concentration (% w/w) |
|---|---|
| 1-methoxy-2-propanol | 30-60% |
| Propylene glycol monomethyl ether acetate | 5-15% |
| 1-ethoxy-2-propanol | 30-60% |
| 1-hexanol | 20-40% |
| 1-octanol | 20-40% |

The listed solvents are preferable, but not limited to only the initiators in TABLE 21.

The total percentage of solvent was then about 25-60% w/w of the whole oligomer mixture. The mixture is stirred, and heated to the initiator's activation temperature, and then kept at 70-90° C. for a controlled period of time from 30 minutes to two hours, until the solution is sufficiently viscous. The oligomer's viscosity is then confirmed to be 2000-3000 cP and is remade if the viscosity is outside this range.

Once the oligomer is prepared, it is incorporated into a mixture containing a collection of monomers. Preferable monomers polymerize through a chain reaction, and therefore contain a C═C double bond in their structure. Preferably, such monomers that are liquid at ambient temperature, and that are hydrophilic and can absorb water in their structure when polymerized can be used in the production of Masterbatch. Specifically, a polymerizable polyethylene glycol (PEG) monomer must be included in the Masterbatch. The PEG monomer chosen may affect the ability of the lens to absorb and release HA. Each monomer, depending on its molecular weight or its end-group, may create larger or smaller pores, which would directly affect the release rate of HA from the lens. Preferable PEG monomers and their concentrations are included in TABLE 22.

TABLE 22
Preferable PEG Monomers for use in Masterbatch

| Monomer | Concentration (% w/w) |
|---|---|
| PEG Diacrylate (M_{w:} 250-6,000) | 2-10% |
| PEG Dimethacrylate (M_{w:} 550-20,000) | 2-10% |
| PEG methacrylate/acrylate (M_{w:} 575-20,000) | 2-10% |
| n-PEG-acrylate (M_{w:} 5,000-20,000) | 2-10% |
| PEG divinyl sulfone (M_{w:} 2,000-5,000) | 2-10% |
| PEG diacetylene (M_{w:} 1,000-3,000) | 2-10% |

The listed PEG monomers are preferable, but not limited to only the monomers in TABLE 22

Preferable monomers and concentrations for the other components of masterbatch are included in TABLE 23.

TABLE 23
Preferable Monomers and Concentrations
for Masterbatch Preparation

| Monomer | Concentration (% w/w) |
|---|---|
| HEMA (hydroxyethyl methacrylate) | 20-60% |
| DMA (dimethylacrylamide) | 2-10% |
| GMA (glycerol monomethacrylate) | 5-20% |
| PVA (polyvinylalcohol) | 5-20% |
| NVP (N-Vinyl Pyrrolidinone) | 10-30% |
| PEG monomer (Table 5) | 2-10% |
| MAA (methacrylic acid) | 2-10% |
| MM (methyl methacrylate) | 2-10% |
| Silicone | 3-10% |
| Siloxane | 3-10% |

The listed monomers are preferable, but not limited to only the monomers in TABLE 23.

The monomers and oligomer are mixed together at about an 80:20 w/w ratio. The mixture was magnetically stirred until a clear solution formed. This monomer/oligomer mixture is called the Masterbatch.

A microemulsion was prepared. This microemulsion was prepared by mixing together an aqueous phase containing water at 5-20% w/w in the microemulsion, and a surfactant or surfactants. Preferable surfactants have an HLB value of 8-18 such as to form an oil-in-water microemulsion. Preferable surfactants are nonionic surfactants with the stated HLB value. Such surfactants are included in TABLE 24:

TABLE 24
Preferable Surfactants and Concentrations
for Microemulsion Preparation

| Surfactant | Concentration (% w/w) |
|---|---|
| Polysorbate 80 | 30-60% |
| Polysorbate 20 | 30-60% |
| Polysorbate 60 | 30-60% |
| Poloxamer 407 | 5-20% |
| 1,3-propandiol | 30-60% |
| surfynol CT 121 | 10-30% |
| Polyethylene glycol 400 | 40-70% |

The listed surfactants are preferable, but not limited to only the surfactants in TABLE 24.

The total percentage of surfactant(s) is 20-60% w/w in the microemulsion. Then, the solution was mixed with an oil until the solution is clear—this signifies that the microemulsion has formed. An oil can be any substance that is not miscible with water without a surfactant. Preferable oils are fatty acids, and examples are included in TABLE 25.

TABLE 25
Preferable Oils and Concentrations for Microemulsion Preparation

| Oil | Concentration (% w/w) |
|---|---|
| Glyceryl Monocaprylate | 2-10% |
| Octanoic Acid | 2-10% |
| Stearic Acid | 2-10% |

The listed oils are preferable, but not limited to only the oils in TABLE 25.

The two components (aqueous phase, oil) are mixed together until a homogenous microemulsion is formed.

Hyaluronic Acid (HA) was mixed with a nonionic surfactant or surfactants such as Tween-20, Tween-80, or Polyethylene Glycol 400 by triturating together in a roughly 70:30 w/w ratio until a white amorphous paste is formed. This combination prevents HA from clumping together as much during the formulation of UV-coating. The combination is called Ink-HA. The combination of HA and surfactant is believed to help incorporate HA into the oily phase of the microemulsion, which then helps to incorporate HA into the UV-coating (though applicant does not intend to be limited to, and is not limited to, a particular mechanism of action).

Masterbatch, Ink-HA, and the microemulsion were mixed together to make UV-ME-coating. Ink HA is added in a ratio that makes HA at 0.25-2.5% w/w of the whole formulation, and microemulsion is added to be 5-15% w/w of the whole formulation. Masterbatch makes up the rest of the formulation. The coating is homogenized until a uniform coating is produced, and the viscosity of the coating is then measured, and confirmed to be 10-30 cP. If it is too high, a solvent such as, but not limited to, 1-methoxy-2-propanol, can be added to the coating to lower the viscosity. Then, an initiator or initiators are added to the solution. Preferable initiators are solid or liquid and can dissolve into/are miscible with the UV-coating. More preferable initiators are UV-light activated, and these initiators and their concentrations are included in TABLE 26.

TABLE 26
Preferable Initiators for Preparation of UV-ME-Coating

| Initiator | Concentration (% w/w) |
|---|---|
| Irgacure 184 | 2-10% |
| Irgacure 500 | 2-10% |
| Irgacure 1300 | 2-10% |
| Darocur 1173 | 2-10% |
| Irgacure 907 | 2-10% |

The listed initiators are preferable, but not limited to only the initiators in TABLE 26.

The coating is then loaded into the inkjet printer, and 2 mg of this coating is printed onto dry hydrogel contact lenses via nozzles in the inkjet printer Xaar XJ-500 printhead, in an inkjet printer setup. The printed-on-contact lenses are then cured under a UV-emitting lamp at specific wavelengths from 210-400 nm, in order to polymerize the coating via a chain reaction. These lenses are cured for 2-5 minutes, so as not to let HA possibly degrade. These printed-on dry contact lenses are then hydrated in a 0.5% w/w NaHCO₃ aqueous solution, and steam sterilized in a phosphate buffered packaging solution containing HA in a concentration of 0.1-0.3% w/w. Afterwards, the lenses were allowed to equilibrate in this packaging solution for at least three days at ambient conditions. The microemulsion-coating contact lenses were prepared in this fashion.

The lenses were hydrated and steam sterilized and allowed to equilibrate for three days in a phosphate-buffered packaging solution containing HA, in order to load the PEG hydrogel with HA on the lens coating. After equilibration, the lenses were tested for HA release in an in-vitro setting. Lenses were removed from the packaging solution, rinsed with 0.9% NaCl to remove excess packaging solution, and extracted in vials containing 2 mL of simulated tear fluid (STF). The STF was sampled according to TABLE 27:

TABLE 27
In-vitro Flux Samples Timepoints:

| Code Name | Number of Lenses | Time Point (h) |
|---|---|---|
| 1-0.25 | 3^{a} | 0.25 |
| 1-1 | 3^{a} | 1 |
| 1-3 | 3^{a} | 3 |
| 1-6 | 3^{a} | 6 |
| 1-24 | 3^{b} | 24 |
| 1-48 | 3^{b} | 48 |
| 1-72 | 3^{b} | 72 |
| 1-96 | 3^{b} | 96 |
| 1-120 | 3^{b} | 120 |
| 1-144 | 3^{b} | 144 |
| 1-168 | 3^{b} | 168 |
| 1-192 | 3^{b} | 192 |

^{a}A set of three lenses were used for each of these time points. At 1 hour, 3 hours, 6 hours, and 12 hours, 100 μL of STF was withdrawn and replaced with 100 μL fresh STF. The removed aliquot was diluted by a factor of 10.
^{b}One set of same three lenses were used for these time points. At 24 hours, 48 hrs, 72 hrs, 96 hrs, 120 hrs, 144 hrs, 168 hrs, 2.0 mL of STF were withdrawn and transferred into glass containers from each of the lens and 2.0 mL of fresh STF was added to vials containing lenses for 48 hours, 72 hours, 96 hrs, 120 hrs, 144 hrs, and 168 hrs time points.

Once all the samples had been collected, they were tested for HA using ELISA assay kit purchased from Echelon Biosciences to quantitate HA released. The results are shown below in TABLE 28:

TABLE 28
Cumulative HA assay for HA Lenses In-Vitro Samples

| Time (hrs) | Microemulsion coated HA Lens -HA Release (ng) |
|---|---|
| 1 | 5933.92 |
| 3 | 6400.15 |
| 6 | 6782.15 |
| 12 | 7280.27 |
| 24 | 7487.13 |
| 48 | 7614.84 |
| 72 | 7688.83 |
| 96 | 7730.15 |
| 120 | 7730.15 |
| 144 | 7765.61 |
| 168 | 7805.98 |
| 192 | 7865.4 |

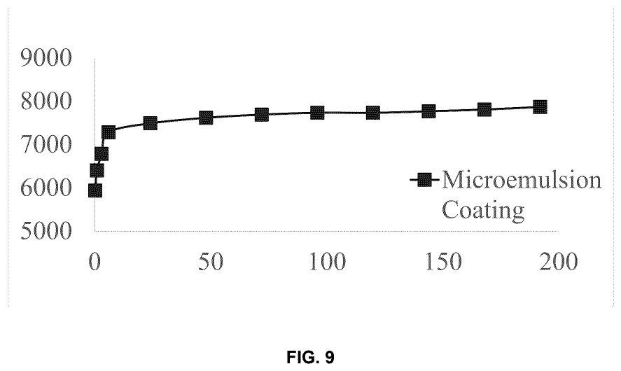

The data was graphed, and the plot is shown in FIG. 9:

The microemulsion HA lenses released HA for about 192 hrs but at very low level to improve surface wettability and providing comfort after initial burst with release slowing afterwards.

The data indicates that the coating process employed by MediPrint Ophthalmics has a positive effect on the burst release of HA, the total cumulative release of HA on the lens, and the release rate of HA from lenses. The higher burst release of the coated lenses can be attributed to the HA loaded PEG hydrogel present in the coating. This supports that HA is successfully incorporated into the coating, and that the coating has a positive effect on HA cumulative release.

Microemulsion HA lenses were tested again for HA release in an in-vitro setting such as an in-vitro flux study. Lenses were removed from the packaging solution, rinsed with 0.9% NaCl to remove excess packaging solution, and extracted in vials containing 2 mL of simulated tear fluid (STF). TABLE 29 shows In-vitro Flux Samples Timepoints:

TABLE 29
In-vitro Flux Samples Timepoints

| Code Name | Number of Lenses | Time Point (h) |
|---|---|---|
| 1-0.25 | 3^{a} | 0.25 |
| 1-1 | 3^{a} | 1 |
| 1-3 | 3^{a} | 3 |
| 1-6 | 3^{a} | 6 |
| 1-24 | 3^{b} | 24 |
| 1-48 | 3^{b} | 48 |
| 1-72 | 3^{b} | 72 |
| 1-96 | 3^{b} | 96 |
| 1-120 | 3^{b} | 120 |

^{a}A set of three lenses were used for each of these time points. At 1 hour, 3 hours, 6 hours, and 12 hours, 100 μL of STF was withdrawn and replaced with 100 μL fresh STF. The removed aliquot was diluted by a factor of 10.
^{b}One set of same three lenses were used for these time points. At 24 hours, 48 hrs, 72 hrs, 96 hrs, and 120 hrs, 2.0 mL of STF were withdrawn and transferred into glass containers from each of the lens and 2.0 mL of fresh STF was added to vials containing lenses for 48 hours, 72 hours, 96 hrs, and 120 hrs time points.

Once all the samples had been collected, the 24-120 hour samples were tested for HA using ELISA assay kit purchased from Echelon Biosciences to quantitate HA released. The results are shown below in TABLE 30:

TABLE 30
HA Cumulative Release measured by ELISA
for HA Lenses In-Vitro Samples

| Time (hrs) | Coated Microemulsion HA Lens Cumulative HA Release (ng) |
|---|---|
| 24 | 12203 |
| 48 | 12718 |
| 72 | 12889 |
| 96 | 13287 |
| 120 | 13470 |

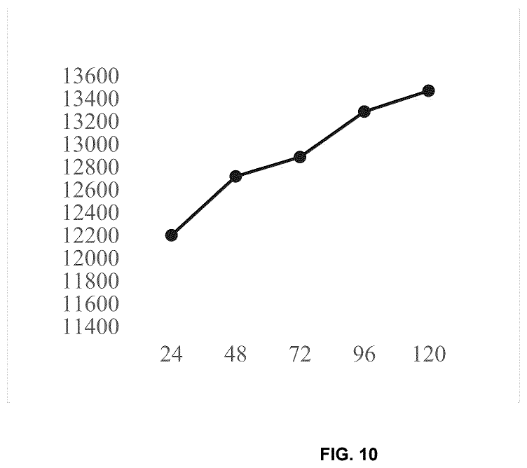

The data was graphed, and the plot is in FIG. 10:

The microemulsion HA lenses released HA continuously for about 120 hrs, which shows that the coated microemulsion lens is capable of releasing HA for up to 5 days (120 hours). This can be attributed to the HA loaded PEG hydrogel, which shows a large burst release of ˜12203 ng HA, and slow release up to 120 hours to a total cumulative release of 13470 ng.

In another experiment, dry hydrogel contact lenses were coated via the coating process and hydrated and sterilized. Here, coated lenses can be referred to by “MPO-produced lenses” or “MPO-coated lenses” or “MPO formulation coated lenses”, where MPO stands for MediPrint Ophthalmics™. Uncoated hydrogel contact lenses of the same variety that were printed on were also hydrated and sterilized. Both lenses were packaged in a HA-containing phosphate-buffered packaging solution and allowed to equilibrate at room temperature for at least three days in order to load HA into the polymerized PEG hydrogel.

Three lenses of each group, coated and uncoated, were tested for HA release in simulated tear fluid. Each lens was removed from its packaging and rinsed in a 0.9% NaCl solution twice for one second each, to remove any excess packaging solution. Then, each lens was added to a contact lens case containing 1.5 mL BioTrue™ multi-purpose solution (MPS), available commercially. BioTrue MPS is known to contain HA as per its package insert, so the lenses were incubated in BioTrue™ MPS in order to test the lenses' ability to retain HA. Lenses were incubated for four hours as per the BioTrue™ MPS package insert. After four hours, the lenses were shaken vigorously to remove excess solution, then added to 2 mL of simulated tear fluid (STF) for HA extraction. Lenses were extracted in STF at 34° C. for 24 hours, and 200 μL samples were pulled at 30 minutes, 1 hour, 2 hours, 6 hours. At each of those timepoints, after 200 μL sample was taken, 200 μL fresh STF was readded, to keep extraction volume at 2 mL. At 24 hours, the sample was taken out of 34° C. All samples were then analyzed by HPLC for HA assay.

The results of HA HPLC Assay from coated vs uncoated contact lenses incubated in BioTrue™ MPS is shown below in TABLE 31:

TABLE 31
HA Assay from MPO-Coated vs Uncoated Contact
Lenses (using MPO-produced HA-contact lenses)

| | | MPO-Coated lens - Avg | Uncoated lens - Avg |
|---|---|---|---|
| | | Release (μg/2 mL) (n = 3) | Release (μg/2 mL) (n = 3) |
| 30 | min | 4.14 | 1.30 |
| 1 | hr | 3.28 | 1.56 |
| 2 | hr | 3.15 | 0.96 |
| 6 | hr | 3.92 | 1.05 |
| 24 | hr | 3.51 | 0.41 |

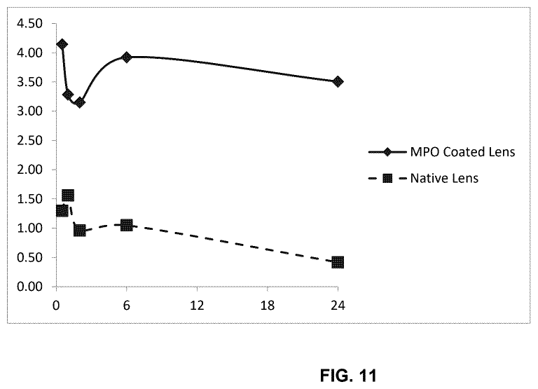

The data in TABLE 31 is graphed in FIG. 11:

The BioTrue™ MPS was measured to contain about 130 μg/mL of HA.

The MPO formulation coated lenses showed a 318% increase in HA assay at 30 minutes, compared to uncoated lenses (4.14 μg in coated, 1.30 μg in uncoated). The data suggests that the MPO formulation coating has a significant effect on increasing HA assay. This can again be attributed to the two factors of the coating: entrapped HA forming a film of HA via hydrogen bonding, and an HA-loaded PEG hydrogel. The hypothesis of HA being loaded into the PEG hydrogel is supported by the increased HA release after BioTrue™ MPS incubation. This shows that HA from the BioTrue™ (MPS) multi-purpose solution was able to reload the contact lenses with HA and the loaded HA on the coated lens can be released.

This study shows that the MPO formulation coated lenses not only had a higher affinity for HA, but also showed an ability to reload HA from incubation in BioTrue™ MPS. Like in the first experiment, it is likely the presence of PEG hydrogel on the coating facilitates an increase of HA loading onto the lens. It was also postulated that entrapped HA in the coating aids in the loading of HA by providing further hydrogen bonding sites, thus forming the film of HA on the lens surface.

### C. Loading and Refreshing Contact Lenses

Microemulsion HA lenses were tested again, to confirm the ability of lenses to be replenished, through soaking in HA solution. Three coated lenses were tested for HA release in simulated tear fluid. Each lens was removed from its packaging and rinsed in a 0.9% NaCl solution twice for one second each, to remove any excess packaging solution. Then, each lens was added to a clean 10-mL glass vial, and extracted in vials containing 2 mL of simulated tear fluid (STF). The STF was sampled according to TABLE 27, as above. Then, after 168-hour time point, the lens was added to a vial containing 2 mL HA packaging solution. The lens was allowed to soak overnight at room temperature. Then, the lenses were readded to fresh vials containing 2 mL STF. The vials were extracted again according to the timepoints in TABLE 27. This process was repeated twice more for a total of four weeks. All samples were tested by ELISA.

The results of each week of HA extraction are shown below in TABLE 32:

TABLE 32
Cumulative HA Release for Weeks
1-4, Weekly HA Replenishment

| | Week 1 | Week 2 | Week 3 | Week 4 |
|---|---|---|---|---|
| | (ng/lens) | (ng/lens) | (ng/lens) | (ng/lens) |
| Day 1 | 9000 | 21791 | 17654 | 20540 |
| Day 2 | 9120 | 22834.2 | 18601.7 | 21420 |
| Day 3 | 9153 | 23156.9 | 19047.9 | 22244 |
| Day 4 | 9179 | 23451.6 | 19399.2 | 22966 |
| Day 5 | 9211 | 23643.9 | 19760.7 | 23671 |
| Day 6 | 9231 | 24044.9 | 21300.7 | —* |
| Day 7 | 9266 | 24394.9 | 22920.9 | —* |

*samples lost, so no datapoint is shown

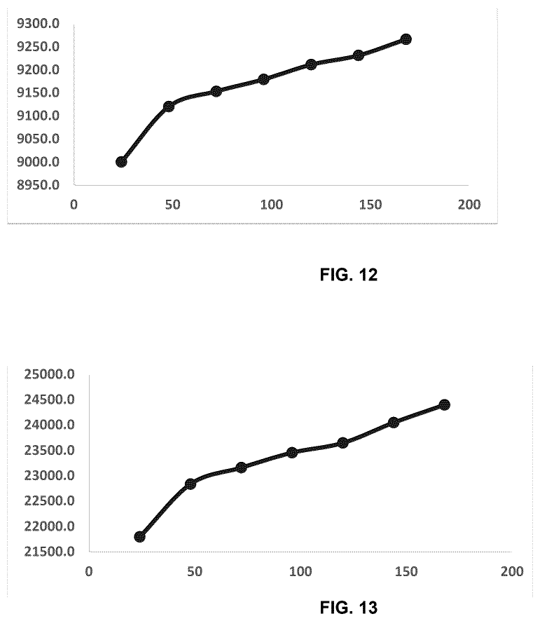

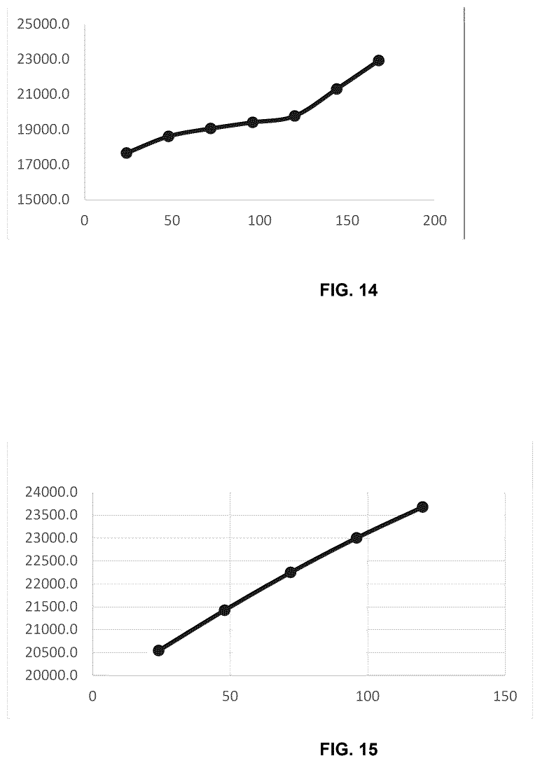

The data of TABLE 32 is shown in FIG. 12 through FIG. 15:

The results of the experiment show that the lens can be replenished through soaking in an HA-containing solution. This confirms the previous experiment's results that the lens may be replenished through soaking in HA solution. This has the further implication that a lens may be used for at least four weeks, through HA soaking each week.

The molecular weight of HA released was also evaluated. Three coated lenses were tested for HA release in simulated tear fluid. Each lens was removed from its packaging and rinsed in a 0.9% NaCl solution twice for one second each, to remove any excess packaging solution. Then, each lens was added to a clean 10-mL glass vial, and extracted in vials containing 2 mL of simulated tear fluid (STF). The STF was sampled according to TABLE 27, as above. Then, each STF timepoint was tested on HPLC, using size-exclusion chromatography. Then, using the retention time of each sample, the molecular weight was calculated using a calibration curve of known molecular weight standards.

The data is below in TABLE 33:

TABLE 33
Molecular Weight of Released HA over One Week

| Time (hrs.) | Molecular Weight (Da) |
|---|---|
| 1 | 692,000 |
| 4 | 709,000 |
| 24 | 575,000 |
| 48 | 539,000 |
| 72 | 384,000 |
| 96 | 301,000 |
| 120 | 200,000 |
| 144 | 51,000 |
| 168 | 49,000 |

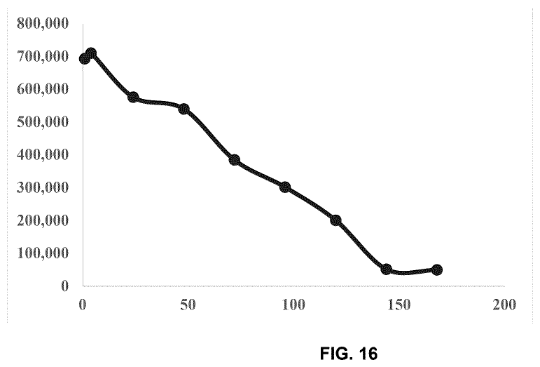

The data in TABLE 33 is graphed in FIG. 16.

The above results show that the molecular weight released during the week Starts around 700,000 dalton on day 1 to about e 50,000 Daltons on day seven. The result is preferable, as the range of high molecular weight HA (50,000 to 700,000 dalton) has greater therapeutic effect than normally expected lower molecular weight range like 500 to 1000 dalton.

### D. Method of Using Contact Lens Having Slow Release of Hyaluronic Acid

Microemulsion-coated lenses, containing HA and Bimatoprost (BMT), a glaucoma drug, were tested in two clinical trials. For both trials, the lenses were worn for three weeks, and a new lens was inserted into each eye once a week for a total of three sets of lenses. The lenses were found to be safe and tolerable, and no protein deposits were found on any of the lenses.

Notably, there were no protein deposits found in any contact lens of any subject. This represents a significant improvement from conventional contact lenses, as “An estimated 30% of all after-care visits by contact lens wearers to optometrists are reported to be due to contact lens deposition” (Beek et al., 2007). Furthermore, no study participant discontinued the study due to contact lens discomfort. This is an important finding, as it was found that “In a study examining reasons for discontinuation from contact lens wear, discomfort was cited as the principal reason by 51% of the respondents” (Beek et al., 2007). HA delivered by the lens may have a significant effect, and the findings support the effect of HA release. The findings also indirectly support the hypothesis that a thin HA film is formed on the lens, due to lack of protein deposits.

Patients also evaluated their feelings of dryness and discomfort while wearing the HA lens via the CLDEQ-8. Patients consistently scored the coated HA lens as being more comfortable and less dry feeling than comparable uncoated lenses. This supports the claim that HA is entrapped in the coating matrix of the lenses, and that HA is releasing for at least one week from the PEG hydrogel.

### E. Preferred Aspects of the Present Invention for Contact Lenses Having Slow Release of Comfort Enhancing Agents

Contact lenses have traditionally had issues with user comfort. Contact lenses that provide comfort enhancers suffered from certain defects as provided below. The present invention addresses this issue and provides additional benefits as well.

For example, Doshi et al. in their patents U.S. Pat. Nos. 9,931,296, 10,413,506, 11,510,869, and 11,234,927 and others in the drug delivery from contact lenses area provide modulation of HA and/or other drugs release through creation of multiple layers of reservoir and barrier layers of different sizes, shapes and structures.

Previous lenses provides conditions where sufficient HA and/or other drugs would not be retained through the process of filtration, hydration, and extraction to provide sustained delivery from 8 hours to more than 7 days.

Previous lenses also provided for reloading of the HA and/or other drugs from packaging solution, but their release would be burst and faster from the lens surface due to adsorption. Thus failing to provide sustained delivery of more than 8 hrs. to 7 days.

Previous lenses also did not provide high molecular weight of HA as it r educes significantly when it goes through the ink manufacturing process step like grinding, filtration, polymerization, wet processing and sterilization. Thus, desired high molecular weight between 10,000 Da and 3 million Da during sustained delivery of HA would not be achieved.

Previous lenses also required incorporation of drug in the ink prior to digital printing, thus requiring stringent manufacturing control to make sure no two pharmaceutical drugs get mixed in a manufacturing line to avoid potential health hazard.

The present invention addresses these limitations and provides additional benefits as well. For example, release of HA is regulated/modulated to provide sustained delivery of HA from 8 hrs to more than 7 days, from a single coating by creating different size pockets (such as, for example, inside the coating film) and pores (such as, for example, on outside of coating film) through fast polymerization in less than 30 minutes (preferably between 1 min and 15 min.) and cross linking of different PEG monomers with different monomers present in monomeric ink. Such pockets/pores of different sizes release HA over a longer period of time but also accommodate HA of high molecular weight during its sustained delivery.

Additionally present invention also offers modulation of drug release rate by creating different strength Hydrogen bond of HA with different hydrogel monomers like HEMA, GMA, MAA, PVP, silicone monomers, macromoric siloxane like TRIS, PDMS etc. to release HA at different times to provide sustained delivery over a long period of time,

The present invention not only facilitates reloading of HA and/or other drugs during or after sterilization from packaging solution but also offers opportunity to replenish with HA and or other drugs from refreshing solution that may or may not be the same as packaging solution.

The present invention provides sustained delivery of HA/drug by adsorbing and absorbing from packaging and/or refreshing solution without incorporating it in the ink. Thus, providing manufacturing friendly process where incorporation of drug is not a part of digital printing and wet lens processing. It is incorporated in packaging solution when digitally printed lens with ink of the present invention is placed in blister pack to load drug from it. The packaging solution may be modified with surfactant for example Trehalose with cyclosporin A drug for dry eye, pH, molality, HA of different molecular weights to get the desired product features. Such product is preservative free due to terminal sterilization. It may be accompanied by additional blisters with just refreshing solution to facilitate replenishment & reuse after it is used.

Using the technologies of the present invention: 1) PEG-rich coatings can offer superior HA retention and sustained hydration for comfort due to stronger H-bonding and hydrophilicity; 2) HEMA-based matrices may offer HA crosslinking or copolymers to prevent rapid diffusion; 3) GMMA offers multiple bonding sites without excessive rigidity; 4) MAA can be useful in pH-responsive systems but may require buffering to maintain HA compatibility.

Preferable monomers and concentrations for the other components of masterbatch are included in TABLE 23.

### F. Preferred Aspects of the Present Invention for Contact Lenses Where Drug is Loaded On/In the Lens From Packaging/Loading/Refreshing Solutions

A digitally printed coating on the surface of contact lenses with or without HA that, when soaked in a drug solution (such as, for example, hyaluronic acid (HA), Trehalose or other ophthalmic agents like Bimatoprost, Timolol, cyclosporin etc), enables sustained, controlled release of the therapeutic over time. This method leverages: 1) Precision surface architecture: Micro-patterned coatings that modulate drug uptake and release kinetics; 2) Soaking simplicity: a manufacturing friendly loading method that avoids handling of drug through manufacturing steps like digital printing drug, or hydration and extraction of drugs 3) Modular adaptability: Compatible with various drugs and lens types, including weekly disposables.

In another example, microemulsion HA lenses were manufactured and sterilized in a bimatoprost-containing packaging solution, to evaluate the HA lens' ability to uptake and release BMT. The packaging solution composition is as follows in TABLE 34:

TABLE 34
Bimatoprost-packaging solution Components

| Component | % (w/w) |
|---|---|
| NaCl | 0.0-5.0 |
| KCl | 0.0-5.0 |
| H₃PO₄ | 0.0-5.0 |
| NaH₂PO₄ | 0.0-5.0 |
| Na₂HPO₄ | 0.0-5.0 |
| Na₃PO₄ | 0.0-5.0 |
| KH₂PO₄ | 0.0-5.0 |
| K₂HPO₄ | 0.0-5.0 |
| K₃PO₄ | 0.0-5.0 |
| Bimatoprost | 0.0-0.05% |
| Sodium Hyaluronate | 0.0-5.0 |
| Water | 95.0-100.0 |

The listed components are preferable, though not limited to the above.

Lenses were removed from the packaging solution, rinsed with 0.9% NaCl to remove excess packaging solution, and extracted in vials containing 5 mL of methanol. After four days of extractions, the vials were opened, and samples were tested by HPLC for BMT assay. The results are shown below in TABLE 35:

TABLE 35
BMT Release from HA-Coated Lens Manufactured
in Bimatoprost-Packaging Solution

| # | Avg Loading (μg/lens) |
|---|---|
| Lens 1 | 38.47 |
| Lens 2 | 28.20 |
| Lens 3 | 27.61 |

The lenses had an average BMT assay of 31.4 μg/lens. This compares favorably to the LL-BMT1 assay value label claim of 32 μg/lens, which was used in a clinical trial. There is a 1.9% difference in the two assay values, so it can be reasonably concluded that LL-BMT1 can be manufactured by using HA-microemulsion lenses and a Bimatoprost-containing packaging solution.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A packaged medical device, comprising:
a) at least one drug delivery contact lens, comprising:
1) at least one coating provided on at least one surface of said drug delivery contact lens;
a) wherein said at least one coating comprises at least one polymerized PEG monomer;
b) further wherein said at least one coating comprises at least one drug;
b) at least one packaging solution, comprising:
1) at least one of said at least one drug;
c) at least one packaging, comprising:
1) said at least one drug delivery contact lens; and
2) said at least one packaging solution.

2. The packaged medical device of claim 1;
wherein said at least one drug is provided in a pharmaceutically effective amount.

3. The packaged medical device of claim 1;
wherein said at least one drug comprises an antibiotic, an intraocular pressure reducing agent, a comfort enhancing agent, an anti-inflammatory agent, a penetration enhancer, a macular degeneration agent, or a combination thereof.

4. The packaged medical device of claim 1;
wherein said at least one drug comprises gentamicin, tobramycin, erythromycin, polytrim, cirproflizacin, viamox, xymar, moxifloxacin, gatifolxacin or a combination thereof.

5. The packaged medical device of claim 1;
wherein said at least one drug comprises timolol, alphagan, axopt, cosopt, lumigan, travatan, xalatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

6. The packaged medical device of claim 1;
wherein said at least one drug comprises sodium hyaluronate, hyaluronic acid (HA), cyclosporine, polyethylene glycol 400, hypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

7. The packaged medical device of claim 1;
wherein said at least one drug comprises, perdforte, lotemax, fluromethlone, nevanac, acular, xibrom, or a combination thereof.

8. The packaged medical device of claim 1;
wherein said at least one drug comprises, Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

9. The packaged medical device of claim 1;
wherein said at least one drug comprises EYLEA® (aflibercept).

10. The packaged medical device of claim 1;
wherein said at least one drug comprises dexamethasone, pilocarpine nitrate, tropicamide, methyl prednisolone, flurbiprofen, penicillin ciprofloxacin, sulphacetaminde sodium, indomethacin, hydrocortisone, indomethacin, ciprofloxacin hydrochloride, insulin, indomethacin, ketorolac tromethamine, or a combination thereof.

11. The packaged medical device of claim 1;
wherein said at least one drug comprises atropine.

12. The packaged medical device of claim 1;
wherein said at least one drug comprises HA, bimatoprost, or a combination thereof.

13. The packaged medical device of claim 1;
wherein said at least one drug comprises hyaluronic acid (HA).

14. The packaged medical device of claim 13;
wherein said HA has a molecular weight between about 5 kDa and about 4,000 kDa.

15. The packaged medical device of claim 13;
wherein said HA has a molecular weight between about 500 kDa and about 2,500 kDa

16. The packaged medical device of claim 13;
wherein said HA is not modified.

17. The packaged medical device of claim 13;
wherein said HA is modified.

18. The packaged medical device of claim 17;
wherein said modified comprises adding at least one functional group to said HA.

19. The packaged medical device of claim 18;
wherein said at least one function functional group comprises methacrylate, acrylate, carboxymethyl, trimethylsilyl, thioethyl, butandiol-diglycidyl ether, divinyl sulfone, amidated HA, tyramine HA, hydrazide HA, thiolated HA, glycidyl methacrylate-HA, or a combination thereof.

20. The packaged medical device of claim 13;
wherein said HA is capable of binding water at between about 4 grams and about 5 grams of water per gram HA.

21. The packaged medical device of claim 13;
wherein said HA has an intrinsic viscosity of between about 0.5 and about 3 m³/kg.

22. The packaged medical device of claim 13;
wherein said HA is steam sterilizable.

23. The packaged medical device of claim 1;
wherein said at least one packaging solution comprises an antibiotic, an intraocular pressure reducing agent, a comfort enhancing agent, an anti-inflammatory agent, a penetration enhancer, a macular degeneration agent, or a combination thereof.

24. The packaged medical device of claim 1;
wherein said at least one packaging solution comprises gentamicin, tobramycin, erythromycin, polytrim, cirproflizacin, viamox, xymar, or a combination thereof.

25. The packaged medical device of claim 1;
wherein said at least one packaging solution comprises timolol, alphagan, axopt, cosopt, lumigan, travatan, xalatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

26. The packaged medical device of claim 1;
wherein said at least one packaging solution comprises sodium hyaluronate, hyaluronic acid, cyclosporine, polyethylene glycol 400, hypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

27. The packaged medical device of claim 1;
wherein said at least one packaging solution comprises, Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

28. The packaged medical device of claim 1;
wherein said at least one packaging solution comprises, perdforte, lotemax, fluromethlone, nevanac, acular, xibrom, or a combination thereof.

29. The packaged medical device of claim 1;
wherein said at least one packaging solution comprises atropine.

30. The packaged medical device of claim 1;
wherein said at least one drug comprises HA, bimatoprost, or a combination thereof.

31. The packaged medical device of claim 1;
wherein said at least one packaging solution does not include a preservative.

32. The packaged medical device of claim 1;
wherein said at least one drug comprises hyaluronic acid (HA).

33. The packaged medical device of claim 32;
wherein said HA has a molecular weight between about 5 kDa and about 4,000 kDa.

34. The packaged medical device of claim 32;
wherein said HA has a molecular weight between about 500 kDa and about 2,500 kDa

35. The packaged medical device of claim 32;
wherein said HA is not modified.

36. The packaged medical device of claim 32;
wherein said HA is modified.

37. The packaged medical device of claim 36;
wherein said modified comprises adding at least one functional group to said HA.

38. The packaged medical device of claim 37;
wherein said at least one function functional group comprises methacrylate, acrylate, carboxymethyl, trimethylsilyl, thioethyl, butandiol-diglycidyl ether, divinyl sulfone, amidated HA, tyramine HA, hydrazide HA, thiolated HA, glycidyl methacrylate-HA, or a combination thereof.

39. The packaged medical device of claim 32;
wherein said HA is capable of binding water at between about 200 grams and about 1000 grams of water per gram HA.

40. The packaged medical device of claim 32;
wherein said HA has an intrinsic viscosity of between about 0.5 and about 3.0 m³/kg.

41. The packaged medical device of claim 32;
wherein said HA is steam sterilizable.

42. The packaged medical device of claim 1;
wherein said at least one packaging comprises at least one blister pack.

43. The packaged medical device of claim 1;
wherein said at least one packaging comprises at least one vial.

44. The packaged medical device of claim 1:
wherein said at least one coating is made in whole or in part by at least one additive printing.

45. The packaged medical device of claim 44,
wherein said at least one additive printing comprises inkjet printing.

46. The packaged medical device of claim 44,
wherein said at least one additive printing comprises digital printing, 3D printing, digital 3D printing, or a combination thereof.

47. The packaged medical device of claim 44;
wherein said at least one additive printing comprises additive 3D printing.

48. The packaged medical device of claim 1;
wherein said at least one coating comprises one or more layers.

49. The packaged medical device of claim 1;
wherein said at least one coating has a viscosity between about 10 and about 35cpscP.

50. The packaged medical device of claim 1;
wherein said at least one coating is made by at least in part by polymerizing.

51. The packaged medical device of claim 50;
wherein said polymerizing comprises photopolymerizing.

52. The packaged medical device of claim 51;
wherein said photopolymerizing comprises UV curing.

53. The packaged medical device of claim 52;
wherein said UV curing is for between about 1 and about 15 minutes.

54. The packaged medical device of claim 1;
wherein said at least one coating is made using filtered materials.

55. The packaged medical device of claim 54;
wherein said filtered materials are made using a filter having a pore size between about 1 and about 10 microns.

56. The packaged medical device of claim 1;
wherein said at least one coating has at least one of said at least one drug dissolved within it.

57. The packaged medical device of claim 1;
wherein said at least one coating has at least one of said at least one drug suspended within it.

58. The packaged medical device of claim 1;
wherein said at least one coating has said at least one drug loaded within it using microemulsion, micelles, encapsulation, nanoparticle, spray drying, in solvent, microspheres, liposomes, ethosomes, niosomes, biodegradable nanoparticles, temperature sensitive smart particles, or a combination thereof.

59. The packaged medical device of claim 1;
wherein said at least one polymerized PEG polymer is made using at least one polymerizable PEG monomer.

60. The packaged medical device of claim 1;
wherein said at least one polymerizable PEG monomer comprises at least one carbon-carbon double bond in its structure.

61. The packaged medical device of claim 1;
wherein said at least one polymerizable PEG monomer is polymerized through free radical polymerization.

62. The packaged medical device of claim 1;
wherein said at least one polymerizable PEG monomer is polymerized by free radical photopolymerization.

63. The packaged medical device of claim 1;
wherein said at least one polymerizable PEG monomer is not biodegradable.

64. The packaged medical device of claim 1;
wherein said at least one polymerizable PEG monomer comprises PEG-diacrylate, mono-PEG acrylate, PEG-diacrylamide, mono-PEG acrylamide, PEG-dimethacrylate, mono-PEG methacrylate, PEG-dimethacrylamide, mono-PEG methacrylamide, n-PEG-Acrylate, n-PEG-acrylamide, PEG diacetylene, PEG divinyl sulfone, or a combination thereof.

65. The packaged medical device of claim 1;
wherein said at least one polymerizable PEG monomer comprises PEG-acrylate, PEG-acrylamide, PEG-methacrylate, PEG-methacrylamide, n-PEG-Acrylate, n-PEG-acrylamide, PEG acetylene, PEG vinyl sulfone, or a combination thereof.

66. The packaged medical device of claim 1;
wherein said at least one polymerizable PEG polymer can absorb a drug from solution.

67. The packaged medical device of claim 1;
wherein said at least one polymerizable PEG monomer is present at a concentration of between about 2 and about 10% w/w.

68. The packaged medical device of claim 1;
wherein said at least one polymerizable PEG monomer is liquid at ambient temperature.

69. The packaged medical device of claim 1;
wherein said at least one polymerizable PEG monomer is molecular weight of between about 200 Da to about 1,000 Da.

70. The packaged medical device of claim 1;
wherein when in use, HA from said drug delivery contact lens is released from about 1 hour to about 30 days.

71. The packaged medical device of claim 1;
wherein when in use, HA from said drug delivery contact lens is released from about 1 hour to about 7 days.

72. The packaged medical device of claim 1;
wherein the pore size of said at least one coating is between about 1 and about 100 nm.

73. The packaged medical device of claim 1;
wherein said packaged medical device, said at least one drug delivery contact lens, said at least one packaging solution, said at least one packaging, or a combination thereof, are sterilized, steam sterilized, or a combination thereof.

74. A medical device, comprising:
a) at least one drug delivery contact lens, comprising:
1) at least one coating provided on at least one surface of said drug delivery contact lens;
a) wherein said at least one coating comprises at least one polymerized PEG monomer;
b) further wherein said at least one coating comprises at least one of said at least one drug; and
2) at least one drug.

75. A method of making a medical device, comprising:
a) providing a contact lens;
b) providing a printable solution comprising:
1) at least one polymerizable PEG monomer;
2) at least one drug;
c) producing at least one coating on at least one surface of said contact lens using at least one additive printing;
d) wherein said contact lens is steam sterilized in its final packaging.

76. A method of using a medical device to treat at least one disease, disorder, or condition of the eye, comprising:
a) providing a subject in need of treatment of a disease, disorder, or condition of the eye;
b) providing at least one drug delivery contact lens of claim 1;
c) operably engaging said at least one drug delivery contact lens with at least one eye of said subject;
wherein said at least one eye of said subject is treated for said at least one disease, disorder, or condition of the eye.

77. A method of using a medical device to treat at least one disease, disorder, or condition of the eye, comprising:
a) providing a subject in need of treatment of a disease, disorder, or condition of the eye;
b) providing at least one drug delivery contact lens of claim 74;
c) operably engaging said at least one drug delivery contact lens with at least one eye of said subject;
wherein said at least one eye of said subject is treated for said at least one disease, disorder, or condition of the eye.

78. A medical device, comprising:
at least one drug delivery contact lens, comprising:
1) at least one coating provided on at least one surface of said drug delivery contact lens;
2) bimatoprost; and
3) N-Vinylbyrrolidone (NVP).
wherein said NVP modulates the release of said bimatoprost from said coating.

79. The medical device of claim 78:
wherein said at least one coating is made in whole or in part by additive printing.

80. The medical device of claim 78;
wherein said at least one coating comprises one or more layers.

81. The medical device of claim 78:
wherein said at least one coating is made at least in part by at least one additive printing.

82. The medical device of claim 81;
wherein said at least one additive printing comprises ink jet printing.

83. The medical device of claim 81;
wherein said at least one additive printing comprises digital printing, 3D printing, digital 3D printing, or a combination thereof.

84. The medical device of claim 81;
wherein said at least one additive printing comprises additive printing, additive 3D printing, or a combination thereof.

85. The medical device of claim 78;
wherein said at least one drug delivery contact lens further comprise at least one additional drug.

86. The medical device of claim 85;
wherein said at least one additional drug is provided in a pharmaceutically effective amount.

87. The medical device of claim 85;
wherein said at least one additional drug further comprises an antibiotic, an intraocular pressure reducing agent, a comfort enhancing agent, an anti-inflammatory agent, a penetration enhancer, a macular degeneration agent, or a combination thereof.

88. The medical device of claim 85;
wherein said at least one additional drug further comprises dorzolamide, timolol, or a combination thereof.

89. The medical device of claim 85;
wherein said at least one additional drug further comprises timolol, alphagan, axopt, cosopt, 13yprome, travatan, 13alatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

90. The medical device of claim 85;
wherein said at least one additional drug further comprises sodium hyaluronate, hyaluronic acid, cyclosporine, polyethylene glycol 400, 13ypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

91. The medical device of claim 85;
wherein said at least one additional drug further comprises perdforte, lotemax, fluromethlone, nevanac, acular, xibrom, or a combination thereof.

92. The medical device of claim 85;
wherein said at least one additional drug further comprises Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

93. The medical device of claim 85;
wherein said at least one additional drug further comprises Afliberset®.

94. The medical device of claim 85;
wherein said at least one additional drug further comprises dexamethasone, pilocarpine nitrate, tropicamide, methyl prednisolone, flurbiprofen, penicillin, ciprofloxacin, sulphacetaminde sodium, indomethacin, hydrocortisone, indomethacin, ciprofloxacin hydrochloride, insulin, indomethacin, ketorolac tromethamine, or a combination thereof.

95. The medical device of claim 85;
wherein said at least one additional drug further comprises gentamicin, tobramycin, erythromycin, polytrim, cirproflizacin, viamox, xymar, or a combination thereof.

96. The medical device of claim 85;
wherein said at least one additional drug further comprises atropine.

97. A contact lens soaking solution, comprising:
a) at least one drug, comprising one or more of:
1) at least one hyaluronic acid (HA);
2) at least one other drug. b) at least one pharmaceutically acceptable carrier.

98. The contact lens soaking solution of claim 97,
wherein said soaking solution is a loading solution for at least one new contact lens.

99. The contact lens soaking solution of claim 97,
wherein said soaking solution is a refreshing solution for at least one used contact lens.

100. The contact lens soaking solution of claim 97;
wherein said at least one drug is provided in a pharmaceutically effective amount.

101. The contact lens soaking solution of claim 97;
wherein said at least one pharmaceutically acceptable carrier is compatible with the eye.

102. The contact lens soaking solution of claim 97;
wherein said at least one other drug comprises an antibiotic, an intraocular pressure reducing agent, a comfort enhancing agent, an anti-inflammatory agent, a penetration enhancer, a macular degeneration agent, or a combination thereof.

103. The contact lens soaking solution of claim 97;
wherein said at least one other drug comprises gentamicin, tobramycin, erythromycin, polytrim, cirproflizacin, viamox, xymar, moxifloxacin, gatifolxacin or a combination thereof.

104. The contact lens soaking solution of claim 97;
wherein said at least one other drug comprises timolol, alphagan, axopt, cosopt, lumigan, travatan, xalatan, combigan, timolol hemihydrate, betaxolol, levobunolol, metipranolol, apraclonidine, Brimonidine tartate, Brinzolamide, methazolamide, dorzolamide, acetazolamide, carbachol, travoprost, latanoprostene bunod, tafluprost, netarsudil, or a combination thereof.

105. The contact lens soaking solution of claim 97;
wherein said at least one other drug comprises sodium hyaluronate, cyclosporine, polyethylene glycol 400, hypromellose, polyvinyl alcohol, carboxymethylcellulose, dextran 70, hydroxypropyl methylcellulose, anhydrous liquid lanolin, mineral oil, white petroleum, mannitol, thiomersal, carbomer, cetrimide, glycerin, polysorbate80, povidine, or a combination thereof.

106. The contact lens soaking solution of claim 97;
wherein said at least one other drug comprises, perdforte, lotemax, fluromethlone, nevanac, acular, xibrom, or a combination thereof.

107. The contact lens soaking solution of claim 97;
wherein said at least one other drug comprises, Flurbiprofen, Acetazolamide, ethylenediaminetetraacetic acid, palmitoyl carnitine, sodium caprate, sodium dodecylsulphate, sodium deoxycholate, poly oxyethylene-g-lauryl ether, 1-α-lysophosphatidylocholine, deoxycholate, taurodeoxycholate, glycocholate, benzalkonium chloride, or a combination thereof.

108. The contact lens soaking solution of claim 97;
wherein said at least one other drug comprises EYLEA® (aflibercept).

109. The contact lens soaking solution of claim 97;
wherein said at least one other drug comprises dexamethasone, pilocarpine nitrate, tropicamide, methyl prednisolone, flurbiprofen, penicillin ciprofloxacin, sulphacetaminde sodium, indomethacin, hydrocortisone, indomethacin, ciprofloxacin hydrochloride, insulin, indomethacin, ketorolac tromethamine, or a combination thereof.

110. The contact lens soaking solution of claim 97;
wherein said at least one other drug comprises atropine.

111. The contact lens soaking solution of claim 97;
wherein said at least one other drug comprises bimatoprost, or a combination thereof.

112. The contact lens soaking solution of claim 97;
wherein said at least one HA has a molecular weight between about 5 kDa and about 4,000 kDa.

113. The contact lens soaking solution of claim 97;
wherein said at least one HA has a molecular weight between about 500 kDa and about 2,500 kDa

114. The contact lens soaking solution of claim 97;
wherein said at least one HA is not modified.

115. The contact lens soaking solution of claim 97;
wherein said at least one HA is modified.

116. The contact lens soaking solution of claim 115;
wherein said modified comprises adding at least one functional group to said at least one HA.

117. The contact lens soaking solution of claim 116;
wherein said at least one function functional group comprises methacrylate, acrylate, carboxymethyl, trimethylsilyl, thioethyl, butandiol-diglycidyl ether, divinyl sulfone, amidated HA, tyramine HA, hydrazide HA, thiolated HA, glycidyl methacrylate-HA, or a combination thereof.

118. The contact lens soaking solution of claim 97;
wherein said at least one HA is capable of binding water at between about 4 grams and about 5 grams of water per gram HA.

119. The contact lens soaking solution of claim 97;
wherein said at least one HA has an intrinsic viscosity of between about 0.5 and about 3 m³/kg.

120. The contact lens soaking solution of claim 97;
wherein said at least one HA is steam sterilizable.

121. The contact lens soaking solution of claim 97;
wherein said HA is modified.

122. The contact lens soaking solution of claim 97;
wherein said modified comprises adding at least one functional group to said HA.

123. The contact lens soaking solution of claim 97;
wherein when in use, said at least one HA from said drug delivery contact lens is released from about 1 hour to about 30 days.

124. The contact lens soaking solution of claim 97;
wherein when in use, said at least one HA from said drug delivery contact lens is released from about 1 hour to about 7 days.

125. A method of loading a new contact lens with at least one drug, comprising:
a) providing at least one new contact lens that comprises PEG;
b) providing at least one loading solution of claim 98;
c) allowing said at least one drug in said at least one loading solution to load into said at least one new contact lens.

126. The method of claim 125,
wherein said at least one new contact lens comprises said drug delivery contact lens of claim 1.

127. The method of claim 125,
wherein said at least one new contact lens comprises HA.

128. The method of claim 125,
wherein said at least one new contact lens does not comprise HA.

129. A method of refreshing a used contact lens with at least one drug, comprising:
a) providing at least one used contact lens that comprises PEG;
b) providing a refreshing solution of claim 99;
c) allowing said at least one drug in said at least one refreshing solution to load into said at least one used contact lens.

130. The method of claim 129,
wherein said at least one used contact lens comprises said drug delivery contact lens of claim 1.

131. The method of claim 129,
wherein said at least one used contact lens comprises HA.

132. The method of claim 129,
wherein said at least one used contact lens does not comprise HA.

133. A loaded contact lens,
made by the method of claim 125.

134. The loaded contact leans of claim 133,
wherein when in use, said loaded contact lens can elute HA a) at a molecular weight of between about 10,000 Dalton to about 3,000,000 Dalton; b) over a time period of more than about 8 hours to about 7 days; or c) combinations thereof.

135. A refreshed contact lens,
made by the method of claim 129.

136. The loaded contact leans of claim 135,
wherein when in use, said loaded contact lens can elute HA a) at a molecular weight of between about 10,000 Dalton to about 3,000,000 Dalton; b) over a time period of more than about 8 hours to about 7 days; or c) combinations thereof.

137. A method of using the loaded contact lens of claim 133, comprising:
a) providing a subject in need of said loaded contact lens;
b) providing at least one loaded contact lens of claim 133;
c) operably engaging said at least one loaded contact lens with at least one eye of said subject.

138. The method of claim 137,
wherein when in use, said loaded contact lens can elute HA a) at a molecular weight of between about 10,000 Dalton to about 3,000,000 Dalton; b) over a time period of more than about 8 hours to about 7 days; or c) combinations thereof.

139. The method of claim 137,
wherein said at least one eye of said subject is treated for at least one disease, disorder, or condition of said at least one eye.

140. A method of using the refreshed contact lens of claim 135, comprising:
a) providing a subject in need of said refreshed contact lens;
b) providing at least one refreshed contact lens of claim 135;
c) operably engaging said at least one refreshed contact lens with at least one eye of said subject.

141. The method of claim 140,
wherein when in use, said refreshed contact lens can elute HA a) at a molecular weight of between about 10,000 Dalton to about 3,000,000 Dalton; b) over a time period of more than about 8 hours to about 7 days; or c) combinations thereof.

142. The method of claim 1,
wherein said at least one eye of said subject is treated for at least one disease, disorder, or condition of said at least one eye.
